SEQUENCE LISTING (78 sequences)

SEQ ID NO: 1 - DNA, 12174 bp, Homo sapiens
  misc_feature 436: y = C or T
gacctacctcgacctttgtgccaggttcttagcatatgggacctgggatggagttagcgctcagttaatagtaactcattagccaggtgcggtggctcatgtctgtattcccagcactttgggagaccgagttgggtggatcacttgagagcaggagtttgagaccagcctggccaacatggcaaaacactatctctaataaaaatacaaaaattagccaggtgtggtggcacttgcctatagtcccagctacacaggaggctggggcagaagaatcacttgaacctgggaggtggaggttgcagtgagccaagattgcaccactgcactccagcctggaaaaaaagggtaattaataactttacttgcaaccatagctgcttctccttctttgagccacccccaatcacccacttagcatccttcaggcctaaayctaggagcagtgcctggtcctctgtcttgttatgaccccaaggaacccacataagagggactgaacattttgctgggcaaggcttccctttgcttgggcagactccactcattctggggctgcagaggcaggaccattcagtcaagctgatgtgggattctgacctaaccaagtccccctccattagtcctcatagcccccacctcccatggggcagccctgagacaggctctgtgacaatccacagcagccctgtccaacagaaccttctgtgatcatggaaacattctgtggctgccaatctggcagccactcgccacatgtgtctatgagccttgaaatgtggccattgtgactgagaaactgaacttttaatggtatttcatttttatttttattttttttttatttattttgaggcagagtctcactctgtcacccgggctggagtgcagtggcactcggctcactgcaagctccgcctcccgggttcacgccattctcctgcctcagcctcgggagtacctgggacaacaggcacccgccaccacgcccggctaattttttgtatttttagtagagatggggtttcaccatggtctcgatctcctgacctcaggtgatccacccgcttcggcctcccgaagtgctgggactgcaggcatgagccaccacgcccggcccagaaaagagatgattaaacataaagcagccatgtgatgaaatggcactttgcctctgtggtcttcctcccccaaacccataactgtaatctaattatgagaaaaacacaggacaattccaatagagagccaggtgcagtggttcacgcctgtaatcccagcactttgggaggctgaggcgggcagatcatgaggtcaagaaatcaagaccatcctggccaacatggtgaaaccccgtctctactaaaaatacaaaaattagctggacgcagtggtgtgcacctgtagtcccagctactcgggaggctgaggcaggagaatcatttgaacccgggaggcagaggttgcagtgagctgagatcgcgccactgcactccagcctggtgacagagtgagactccgtctcaaaaataaataaaaataaataaataaaaattagctgggcgtggtggcacgtgcctgtaatcccagctactcagaggctgaggcacaagaatcacttgaacctgggagacagagattgcagtgagccgagattgtgccactgcactccagcctgggcgacagagtgagactacaacaaacacacacacacacacccacacacacacacacacaaattccaagagagggtcatcctgaccaatactcctcaaaactatcaaggttgctgggcacagtggctcacgcctgtgatcccaatgctttgggaggcttagatgggaggatcacttgaggccaggagttcaagaccagcctgggcaacatagggagacgccgtgtctccaaaaatttttttgagacagagtctcgctgtgtcgcccaggccggagtacagtggcgtgatctcggctcactgcaaactctgcctcctgggttcacgccattcttctgcctcagcctcccaagttgctgagattacaggcacccgccaccatgcccagcttattttttgtatctttagtagagacaaggtttcactgtgttagccaggatggtctccatcacctgacctcgtgatccgcctgcctcggtctccccaagtgctgggattacaggtatgagccaccgtgcctggcccaaaaaatttttttaaattagccaggtgtggtgacacatgtctgtagtccccactaatcgggaggctaaggtgggaggattgcttgagcccaggaggttgaggctgcagtgaactatgatcgtgtcactgcacatcagtctgggaaacagagcgacactttgtctcaaaaaaaaaaaacagataaataaattaaataaccaggccctccttatcccacagggttgttgtagaggtgacataggaacagaagagcaccaagttaaccaattataaatctatatagagagaagcagatcagaggccaggcacagtggctcatgcctataatcccagcattttgggaggctgaggagtggatcacctgaggtcaagagtttgagaccagcctgaccaacatggtgaaaccttgtctctactaaaaatacaaaaattatccaggcatgctggcaggcgcctgtaattcccagctacacgagaggctgaggcaggagaatcgcttgaacctgggaggcggaggttgcagtaagccgagatcgtgccattgcactccagcctgggcgacaagagcgaaactctgtctcaaaaaaaaaagagagagagagagagaagcagattagcagttaccaggggctgagggagtgtgactgctaatgggtacagggtttccttctggagtgataaaaatgttctggaaccccatagaggtgatggttgcacaacactgtgaaggtactaaatgcccccgaattgtttacttaaacgtggttaatgttatgtgaatttcagctaaacaatgttatgtagatatttggccgggcgcggtggctcacgcctgtaatcccagcattttgggaggccgaggcaggtggatcacgaggtcaggagatcgagaccatcctggctaatgcggtgaaaccccatctctactaaaaatacaaaaaaaaaaattaaccgggcgtggtggtgggtgcctgtagtcccagctacttgggaggctgaggcaggagaatggcatgaacctgggaggcagagcttgcagtgagccaagatcgcgccattgcactccagcctaggcaacagagcaagactccgtctcaaaaaatatatataaataaatagatatgtgatgtgacaggtttttttttgagatggagttttgctcttgttccctaggctggagtgcaatggcgtgatctcagctcaccgcaacctccgcctccagggttcaagccattctcctgcctcggcctccggagtagctgggattacaggcataagccaccatgcctggctaattttgtgtttttagtagagacagggttattccatgttggtcaggctggtctcgaactctccacctcaggtgatctgccagcctcagcctcccaaagtgctgggattacaggcatgagccaccgtgcctggcctctgatatgacagttctaatgccctttagtattctataattcagactcaggcctttggaatccaaagcccaggtttttctcacaaacccacactgcagagcggagtggtggaaaaaaataaaacctctgccttggaatcagacagatctaaactggagccctattttgtcatttgccaactgtgtgaccttgggcaagttaccgcaactctctgaacctgtctctttatctgcaaggtgcacgactgatgggactattcaaccagacccagtgcacagattcaggcacttgataagacattgaggctgcaggcagcgatcttttttctttctttctttttttttttttttttttgaaatagggtctcactctgctgcagaggctcaatcactgttcattgcagccttgacctccctggctcamgagatcctcccatctcagcctcctgagttgctgggatcacaggtgcaatccaccaccacacctggttaacatttttttttttagagatgaggtctctctatgttgcccaggctgcacttccttcttgtctcccttatcccagcgtccgactgaactgacggctttgctttccccaaccagcccgtgaagctgggctgagtacaaagtggtgggtatgagggtcaagattgtaagatctgaaaactccagaaaccatccctttggttaacagttgctaaggacaaatgcataacatattttccagtgatcccatgctggcaaatcgtcagggtcattcctgcaacagacagattcaaggccagccccaaactcagccaagagcaaagcaaacactccagccttatctgggcagggttgtgtggagactgactataagactatacctgagactggtcatctcagttcttttctcaccttgactgcaagatgaaactccttgtgctagctgtgctgctcacaggtaggcaagtctccccggctccacccgcctttctctcccaagtgagctaagatctcactcctctggaatgggggccacaggccacagcaaacagggatggccagccccgcagtctcaawtcgaggttcccagtggggcttaagggctcctctattggggttccctcaaggctggcactttttcaacctgcaagtctgaactcagattgcctgagctaagaaagcttgcctttattttcttttttccagacagggtcttgctctatcacccaggctggagttcagtggcatgatcatagctcaccacagcttccaactcgtgggctcaagtgatcctcccaccttactcaactaagtagttaggccaatctcccatttattttattttattttaatttttatttttattttactttattttatttttgagacggggctcactctgtcgcccaggctggagtgcggtggcgtgatctcagatcactacaacctccatctcctgggttcaaataattctcttgcctcagcctctcaagtagctgggacttgtagctctcaagtagctggcacacaccaccatgcccagctaattttttgtgtgttttttttggtagagacaggttttcaccatgttggccaggctgggtgacctcccttttagattctcctcatcctgctctattcttcccctttctaatgcagtatccagtttccttacttatcacatttattattattcttattattattgagacagagtcttgctttgtcgccaaggctggagtacagtggtgcgatctcggctcactgcaagctccacctgctgggttcacgccattctcccgcctcagcctccccagtagctgggactaaaggcgcctgccaccacgccccgctaatttttttgtatttttaataaagacggggtttcatcgtgttagccaggatggtctcgatctcatgaccttgtgatccgcctgcctcggcctcccaaagtgctgggattacaggcatgagccaccgtgcccggccttatcacatttattatttattgtttttctctcccactaggttgtaagctccatgaggttagagattattattattattattattattattattattattattattattatatctgttcactgctgtatctctagctcctaggacagagcctggcacatagtaagtgctcaataaatattcactggataaacagtgcagatagtttaaaactatctgacctagggaggctgaggcaggagaatggcgtgaacccgggaagcagagtttgcagtgagctgaaatcgtgtcactgcactccaacctgggcaacagagcaagactccatctcaaaaaaaaaaaaaaaactatcaggcctagctgggtggcacatgcctgtaatcctagctgaggcggtagggtcccagaagaagaagaagaagaaaaagaagaagatatatatatatatacacacacacaaagatataaactttatatatataaagttttcattaaaaaaaaaaaaaaacctctacccactttcactttaccaggttcctgggtccaacggtcttcagaggaggcagctggcaggggtcagggaggcagcgtgggacccgagggagcaggaaggcagtgtgtccccggggtgctggcagaccgatttgaactctggctatgtcttcttgcagtggccgccgccgmcagcggcatcagccctcgggccgtgtggcagttccgcaaaatgatcaagtgcgtgatcccggggagtgaccccttyttggaatacaacaactacggctgctactgtggcttggggggctcaggcacccccgtggatgaactggacaagtaagtgatccgcctgcaggaaaattggagtgcctgccgggggcggggtggggcacacgccaaggatctcacgaggcatacaaaggggacttgcatatctgctaaggataacatattttcacctcttgtcaaataaacaaatatgttccaagaggaccctgtagcgaacgcaccccgttagagatggaaacaatgaccgacgtgcaaaacagtgggcgatgctgccctccagtggcagaatgtagcaacagtaaacatcacagcaactatccacgtgtcattttctagcagtggttgtcactgcaccttctgaatacaggattttactgtattcttgcaaccatgttaaaaatcgctttcaggccaggcgcggtggctcatgcctgtaatcccagcactttgggaggccgaggcgggcggatcacttgaggtcaggagttcgagaccagcctggccaacatggtgaaaccctgtctctactaaaaaatacaaaaattagccggacatggtggcgagcgcctgtaaccccagctacttgggagactgagttggaggtttcagtgagccaaggtcgtgtcactgctgtccagcctgggtaacagagcaactctgtctcaaaaaaaaaaaatgctttcaataaatatatgataaaaggacttatattttttcaagccataggatcatttctcctgaagcatcttggcgaagtcatccccacctgttcctgagagtgggcaggtgagggctgacctattgctctgcacttactcctatctcagctgtccctcccactttccaggtgctgccagacacatgacaactgctaygaccaggccaagaagctggacagctgtaaatttctgctggacammccgtacacccacacctattcatactcrtgctctggctcggcaatcacctgtagcagtaggtttatcccttccttgacctatgaattctagttggttctcagtaggccggggggaaataatagtaacaacagccatgatttagtgttaattttcttggttctgggcagtgtctcctttaatcctcagaacaacactatgggataggtacaattatcctcacttaacagataagaaaactgaggctcagaaggctgagctatttgcccaagatcacacagcttgtaagtggtgacagtttgggtttttttttgttgttgtttagagacagggtcttgctctgtcacccaggcatgagcacagtggtgcaaccataggtcactgcagcctcaacctcctgagctcaagggatctgctgacctcagcctcccaagtagctgggactacgagcgtgcaccaccacgcctggctaattaaaaaaatttttttgtagagactgggtcttactacgttggccaggcttgtcttaaactcctggcttcaagcaatcctcctaccttggcatcccaaagtgctgggattacaggggtgagccaccatgtgcggctacttatttctttacattccatctttccaatagaatgtaagatccacagaacagggattactgcctattttcttcctttcttttttgagacagagtctcacttcatcacctcaacctccgttcagctcactgcaacctctgcctcccgggttcaagygattctcctgcctaagcctcctgagtagctggaattacaagcgtgcaccaccatgcttggctaattttttgtatttttagcagagatggggttttaccatgttgcccaggctggtctcaaactcctgacctcaagtgatctgcctgcctcagtctcccaaagtgctggaattataggcgtgagtcactgtgcctggccgattactgtctattttctttattgctatatccccagatctagagcagtgtctgacatatagtaggtgctcaataaataattgatgaatgcacagcctagatataaactttctttttctttttttaaaacaatcttgacaactttgcagaataaatacaatcttgcattctgctttttcacttatcaccttgttatgactttttcatattgcctcaaacctttattgttactgttttttcattgttactattttagtcactgaataatatggcttaatttgcttatacatcctcctgctccactttagaaggccaaatttacaaatctgatgaaagctatgaaccctctccccagagaaatacacacacacacacacactcacacacagtttttttttaatgtttgcaactaagacaagaaacctgcattagaggatgtttgttcatattaattaaaaataactcagttgggcacagtgactcaagcctgtaaccacagtactttggaagtccaaggtgggtggatcacttgaggtgagaagttcgagaccagcctggtcaatatggtgaaaccctatctctactaaaaatacaaaaattagctgggtgtagtgatgcatgcctgtagtcccagctactcgggaggctgaggcaagagaattgcttgaacctgggaggcagaggttgcagtgagccgagatcccaccactgcactccagcctgggcgacacagcgagactctatctcaaaaaaataaataaataaaataaaggatcggagagaaacaaaactaataagattcctgaaggtaagcagagatacgtaaattatatgtaataaagtttaaatgcattttaactgtaatcttattgtttattttggttataaaagtaaacaagccaaaagtaatgcaacttcaaackctacataaatatctattatggaaagtggaaggcatctataatcctactacccaaagataaccagttacatattcctccagatttttggggcatacactagctttttttatttgggaaaatttccatgtgcaggcatacctaatttttctaaatgtctatgtagtattccatttaaggatgttccataatttttaaaatacatgctttaaagtagagaaactaggttgggcatggtggctcacgcctgtatcccagcactttgggaggccgaggcaaatggatcacttgaggtccggagtttgagaccagcctggacaacatgatgaaacaccctctctaataaaaatacaaaaattagctgggcatggtggcaagcacctgtagtcccagctactcaggagtctgaggcaggagtatcacttgaactcaggaggcagaagttgcagtgagctgagatcacgccactgcactccagcctaggcgacaaaagggaaactccgtcttaaaaacaaaacaaaacaaaaaaacacaggatgcccagataaatatgactttcagataagcaatggataattttttggggggtatatgtcccaaatattgcattcattgtttatctgaaagtcaaatttaactgggcatcctgatgtacttgtattcacttaatctgtcagccctaaatgtgcatcagtggaatggctgccagcttattccagttaattcttcttgccccagattgtacaaaacagggtccaccttggctcagtcctctcctttcatccctctccaggcaaaaacaaagagtgtgaggccttcatttgcaactgcgaccgcaacgctgccatctgcttttcaaaagctccatataacaaggcacacaagaacctggacaccaagaagtattgtcagagttgaatatcacctctcaaaagcatcacctctatctgcctcatctcacactgtactctccaataaagcaccttgttgaaagacctcatgtttggatattgttttattctctgtctataaactaggtctctgcctactcttttatttttatgtatttattttttctaggtggagtcttgctctgtggcccaggctggagtgcagtgatgccaccttgcctcactgcaacctccgcctcccgggctcaagcaatcctcccgcctcatcctcccgagtagctgggaccataggcatgcaccaccatgcctggctaatttttgtattttttgtagagacagagtttcgccatgttgccctggctggtctcaaactcctcagcttaagtgatctgcctggctcggcctcgcaaagtgttgggattacatgcatgagccgccgcgcctggctactctgcctagtcttttgtgagtatcatttcttccagccttggaagctaagttgaattagaaagacacttccaggaagcaagcaagcaccttgaaacctgagtaatgattaacgatcaccatctactgattatttactctgtaccaggactgtgtgtccataaatcctcttgacagccctgtgaggtattggcgctattagcaaatcttattttcctaagctgaggctcaataggagaggtcacttttccaatgctatcatctagtaagcagcagagaaggaatttgaactcggcaagtctaacaacagaaaacacatgctgaaccactgcccttccctgcctgaagtggtaggctttagtttgagccagaccttgcccccgtctcatgattctgcctccattttcaactgtattaaaccatttttctacaatgactttcttttttttttttttttttgagatggagtctcgctctgtcgcccaggctggagtgcagtgctgcaatctcggctcactgcaagctctgcctcccaggttcacgccattctcctgcctcagcttcccgagtagctgggtttacaggctcctgccaccacgcccagctaattttttgtattttcagtagagacggggtttcaccgtgttagccaggatggtctcgatctcctgacctcgtgatccgcccgcctcggcctcccaaagtgctgggattacaggcgtgagccaccgcacacggccacgactttcttttctaaataaaagacttcaccacactctacaggctaattttgacactgtagtcatgaaatataataaacattaacaagccgagcatggcggcacgcgcctatgatcgtagctactcaagaggctgaggcaggaggatctcttgatcccgggagtttgaggctgcagtgagctatgatcacaccactgcactccagcctgggtgaaagagtgagaccctgtttcaagctactagggaggctgaagtggaaggatcccctgagcccaggagttggaggctgcagtgagctgtgatcacgccactgcactccagcctgagtgacagagagagacactatctcaaacaaacacacacacaaaacmcaaacaaaacaaaacaaaacaaaacaaaacaaaacaaaaaaccaataacagcttgcatttctggagcacttactgcatacttccttgttcggagttttccacatctcatctcattaaatgttcaaaccagctctgtgatattgatatttttgctcccatttcatggatgtggaactaaaaattcagagaagttaagtcatttgtccaagatcacacaaatggcaaaatcaggatttggccaggtctgtctggtggcagtgcccaagcttttaaccactaagtcacttcagcccaattcctctatgagtatttatgactacatttacattgaaattcaccagaactaagccagggacagtggctcacgcctgtaatcccaggactttgagaagtctaggtgggcagatcacttgaggccaggagtttgagaccagcctggccaacatggcaaaaccctgtctctactaaaaaatacaaaaattagccgagtatggtggcataggcctgtaatcccaactactcagg

SEQ ID NO: 2 - protein, 148 aa, Homo sapiens
MKLLVLAVLLTVAAADSGISPRAVWQFRKMIKCVIPGSDPFLEYNNYGCYCGLGGSGTPVDELDKCCQTHDNCYDQAKKLDSCKFLLDNPYTHTYSYSCSGSAITCSSKNKECEAFICNCDRNAAICFSKAPYNKAHKNLDTKKYCQS

SEQ ID NO: 3 - DNA, 562 bp, Homo sapiens
tggtcatctcagtttcttttctcaccttgactgcaagatgaaactccttgtgctagctgtgctgctcacagtggccgccgccgacagcggcatcagccctcgggccgtgtggcagttccgcaaaatgatcaagtgcgtgatcccggggagtgaccccttcttggaatacaacaactacggctgctactgtggcttggggggctcaggcacccccgtggatgaactggacaagtgctgccagacacatgacaactgctatgaccaggccaagaagctggacagctgtaaatttctgctggacaacccgtacacccacacctattcatactcgtgctctggctcggcaatcacctgtagcagcaaaaacaaagagtgtgaggccttcatttgcaactgcgaccgcaacgctgccatctgcttttcaaaagctccatataacaaggcacacaagaacctggacaccaagaagtattgtcagagttgaatatcacctctcaaaagcatcacctctatctgcctcatctcacactgtactctccaataaagcaccttgttgaaagaa

SEQ ID NO: 4 - DNA, 552 bp, Mouse
ctcccctcactccttctgaagatgaaactccttctgctggctgctctgctcacagcaggcgctgctgcacacagcatcagccctcgggctgtgtggcagttccgcaatatgatcaagtgcaccatccccgggagtgatcccctgaaggattacaacaactatggctgctactgtggcttgggcggctggggcaccccagtggacgacttagacaggtgctgccagactcatgaccactgctacagtcaggccaagaagctggaaagctgtaaattcctcatagacaacccctacaccaacacttactcctactcatgctccgggagcgagatcacctgcagcgccaaaaacaacaaatgcgaggacttcatctgcaactgtgaccgtgaggccgccatctgcttctccaaggtcccgtacaacaaggaatacaaaaaccttgacaccgggaaattctgttagcctgtcacctcacttcctgcccacgccgaccccgcccaccttgctgtcttatttcaccctgcgccctctaataaagtacctgctgtcaga

SEQ ID NO: 5 - DNA, 542 bp, rat
ccctcgccaagatgaaactccttctgctggctgctttgctcacagcaggcgttactgcacacagcatcagcactcgggctgtgtggcagttccgcaatatgatcaagtgcaccatccccgggagtgatcccctgagggagtacaacaactacggctgctactgtggcttgggcggctcaggcaccccagtggacgacttagacaggtgctgccagactcatgaccactgctacaatcaggccaagaagctggaaagctgtaaattcctcatcgacaacccctacaccaacacgtactcatacaagtgctccgggaacgtgatcacctgcagcgacaaaaacaacgactgtgagagcttcatctgcaactgtgaccggcaggccgccatctgtttctccaaggtcccctacaacaaggaatacaaagaccttgacaccaagaaacactgttaggctgtcaccccacttcctgtctatgccgtccccgctccccttgctgtcttatttctgcaccgcaccctctaataaagtaccagcagaaag

SEQ ID NO: 6 - DNA, 289 bp, P. obesus
  misc_feature 269: n = A,T,C or G
tgttccgcaatatgatcaagtgcgccatccccggaagtaagcccctgaaggagtacaacaactacggctgctactgcggcctgggcggcgcaggcaccccagtggacgaattagacaggtgctgccagatccatgacaattgctacactaaggccaagaggctgaaaagctgtaaatccctcctggacaacccctacacccactcatactcgtacaagtgctccgggaatgagatcatctgtagtgacaaaaacaaggaatgcgaggcnttcatctgcaactgtgaccg

SEQ ID NO: 7 - protein, 148 aa, Homo sapiens; identical to SEQ ID NO: 2

SEQ ID NO: 8 - protein, 146 aa, Mouse
MKLLLLAALLTAGAAAHSISPRAVWQFRNMIKCTIPGSDPLKDYNNYGCYCGLGGWGTPVDDLDRCCQTHDHCYSQAKKLESCKFLIDNPYTNTYSYSCSGSEITCSAKNNKCEDFICNCDREAAICFSKVPYNKEYKNLDTGKFC

SEQ ID NO: 9 - protein, 146 aa, rat
MKLLLLAALLTAGVTAHSISTRAVWQFRNMIKCTIPGSDPLREYNNYGCYCGLGGSGTPVDDLDRCCQTHDHCYNQAKKLESCKFLIDNPYTNTYSYKCSGNVITCSDKNNDCESFICNCDRQAAICFSKVPYNKEYKDLDTKKHC

SEQ ID NO: 10 - protein, 146 aa, P. obesus
MKLLLLAALLTAGVGAHSISTRAVWQFGNMIKCAIPGSKPLKEYNNYGCYCGLGGAGTPVDELDRCCQIHDNCYTKAKRLKSCKSLLDNPYTHSYSYKCSGNEIICSDKNKECEAFICNCDRAAAICFSKAPYNKQDKNLNTKKNC

SEQ ID NO: 11 - DNA, 20 bp, Artificial Sequence
  primer
tgcagaggctcaatcactgt

SEQ ID NO: 12 - DNA, 19 bp, Artificial Sequence
  primer
caggtgtggtggtggattg

SEQ ID NO: 13 - DNA, 19 bp, Artificial Sequence
  primer
cacaggccacagcaaacag

SEQ ID NO: 14 - DNA, 22 bp, Artificial Sequence
  primer
tcagacttgcaggttgaaaaag

SEQ ID NO: 15 - DNA, 20 bp, Artificial Sequence
  primer
ggcagaccgatttgaactct

SEQ ID NO: 16 - DNA, 17 bp, Artificial Sequence
  primer
cgggatcacgcacttga

SEQ ID NO: 17 - DNA, 19 bp, Artificial Sequence
  primer
ggcagttccgcaaaatgat

SEQ ID NO: 18 - DNA, 20 bp, Artificial Sequence
  primer
tgcaggcggatcacttactt

SEQ ID NO: 19 - DNA, 19 bp, Artificial Sequence
  primer
agctgtccctcccactttc

SEQ ID NO: 20 - DNA, 19 bp, Artificial Sequence
  primer
gtgtgggtgtacgggttgt

SEQ ID NO: 21 - DNA, 19 bp, Artificial Sequence
  primer
agctgtccctcccactttc

SEQ ID NO: 22 - DNA, 22 bp, Artificial Sequence
  primer
ataggtcaaggaagggataaac

SEQ ID NO: 23 - DNA, 19 bp, Artificial Sequence
  primer
agctgtccctcccactttc

SEQ ID NO: 24 - DNA, 22 bp, Artificial Sequence
  primer
ataggtcaaggaagggataaac

SEQ ID NO: 25 - DNA, 20 bp, Artificial Sequence
  primer
caagaagctggacagctgta

SEQ ID NO: 26 - DNA, 22 bp, Artificial Sequence
  primer
ataggtcaaggaagggataaac

SEQ ID NO: 27 - DNA, 20 bp, Artificial Sequence
  primer
atcacctcaacctccgttca

SEQ ID NO: 28 - DNA, 20 bp, Artificial Sequence
  primer
ggtggtgcacgcttgtaatt

SEQ ID NO: 29 - DNA, 26 bp, Artificial Sequence
  primer
aaggtaagcagagatacgtaaattat

SEQ ID NO: 30 - DNA, 26 bp, Artificial Sequence
  primer
ggttatctttgggtagtaggattata

SEQ ID NO: 31 - DNA, 16 bp, Artificial Sequence
  extension oligonucleotide
tgagatgggaggatct

SEQ ID NO: 32 - DNA, 14 bp, Artificial Sequence
  extension oligonucleotide
actgggaacctcga

SEQ ID NO: 33 - DNA, 13 bp, Artificial Sequence
  extension oligonucleotide
gctgatgccgctg

SEQ ID NO: 34 - DNA, 13 bp, Artificial Sequence
  extension oligonucleotide
ggagtgacccctt

SEQ ID NO: 35 - DNA, 17 bp, Artificial Sequence
  extension oligonucleotide
acacatgacaactgcta

SEQ ID NO: 36 - DNA, 15 bp, Artificial Sequence
  extension oligonucleotide
ggtgtgggtgtacgg

SEQ ID NO: 37 - DNA, 15 bp, Artificial Sequence
  extension oligonucleotide
ggtgtgggtgtacgg

SEQ ID NO: 38 - DNA, 18 bp, Artificial Sequence
  extension oligonucleotide
ccacacctattcatactc

SEQ ID NO: 39 - DNA, 16 bp, Artificial Sequence
  extension oligonucleotide
cttaggcaggagaatc

SEQ ID NO: 40 - DNA, 17 bp, Artificial Sequence
  extension oligonucleotide
gtaatgcaacttcaaac

SEQ ID NO: 41 - DNA, 20 bp, Artificial Sequence
  primer
acccacttagcatccttcag

SEQ ID NO: 42 - DNA, 20 bp, Artificial Sequence
  primer
tcttatgtgggttccttggg

SEQ ID NO: 43 - DNA, 20 bp, Artificial Sequence
  primer
tgtggccattgtgactgaga

SEQ ID NO: 44 - DNA, 17 bp, Artificial Sequence
  primer
gcccgggtgacagagtg

SEQ ID NO: 45 - DNA, 20 bp, Artificial Sequence
  primer
tgtggcagttccgcaaaatg

SEQ ID NO: 46 - DNA, 20 bp, Artificial Sequence
  extension oligonucleotide
agtagcagccgtagttgttg

SEQ ID NO: 47 - DNA, 20 bp, Artificial Sequence
  primer
accccgttagagatggaaac

SEQ ID NO: 48 - DNA, 20 bp, Artificial Sequence
  primer
ctgttgctacattctgccac

SEQ ID NO: 49 - DNA, 20 bp, Artificial Sequence
  primer
aatttctgctggacaacccg

SEQ ID NO: 50 - DNA, 20 bp, Artificial Sequence
  primer
cctactgctacaggtgattg

SEQ ID NO: 51 - DNA, 20 bp, Artificial Sequence
  primer
caagccaaaagtaatgcaac

SEQ ID NO: 52 - DNA, 20 bp, Artificial Sequence
  primer
ggattatagatgccttccac

SEQ ID NO: 53 - DNA, 20 bp, Artificial Sequence
  primer
tcatctcacactgtactctc

SEQ ID NO: 54 - DNA, 20 bp, Artificial Sequence
  primer
caatatccaaacatgaggtc

SEQ ID NO: 55 - DNA, 20 bp, Artificial Sequence
  primer
gacagagagagacactatct

SEQ ID NO: 56 - DNA, 20 bp, Artificial Sequence
  primer
gaaatgcaagctgttattgg

SEQ ID NO: 57 - DNA, 21 bp, Artificial Sequence
  ddNTPs
ttagcatccttcaggcctaaa

SEQ ID NO: 58 - DNA, 24 bp, Artificial Sequence
  ddNTPs
gactctgcctcaaaataaataaaa

SEQ ID NO: 59 - DNA, 21 bp, Artificial Sequence
  ddNTPs
gccgtagttgttgtattccaa

SEQ ID NO: 60 - DNA, 22 bp, Artificial Sequence
  ddNTPs
gtgcaaaacagtgggcgatgct

SEQ ID NO: 61 - DNA, 19 bp, Artificial Sequence
  ddNTPs
tgattgccgagccagagca

SEQ ID NO: 62 - DNA, 19 bp, Artificial Sequence
  ddNTPs
tgattgccgagccagagca

SEQ ID NO: 63 - DNA, 24 bp, Artificial Sequence
  ddNTPs
cactgtactctccaataaagcacc

SEQ ID NO: 64 - DNA, 22 bp, Artificial Sequence
  ddNTPs
caaacaaacacacacacaaaac

SEQ ID NO: 65 - DNA, 32 bp, Artificial Sequence
  primer
acgttggatggggttgtccagcagaaatttac

SEQ ID NO: 66 - DNA, 28 bp, Artificial Sequence
  primer
acgttggatgctttccaggtgctgccag

SEQ ID NO: 67 - DNA, 19 bp, Artificial Sequence
  primer
agacacatgacaactgcta

SEQ ID NO: 68 - DNA, 20 bp, Artificial Sequence
  primer
gctgtgtggcagttccgcaa

SEQ ID NO: 69 - DNA, 22 bp, Artificial Sequence
  primer
gttccgcaatatgatcaagtgc

SEQ ID NO: 70 - DNA, 23 bp, Artificial Sequence
  primer
gatgaaactccttctgctggctg

SEQ ID NO: 71 - DNA, 22 bp, Artificial Sequence
  primer
saagatgaaactccttctgctg

SEQ ID NO: 72 - DNA, 20 bp, Artificial Sequence
  primer
ggtgaaataagacagcaagg

SEQ ID NO: 73 - DNA, 20 bp, Artificial Sequence
  primer
ggagaancagatggcggcct

SEQ ID NO: 74 - DNA, 21 bp, Artificial Sequence
  primer
cggtcacagttgcagatgaag

SEQ ID NO: 75 - DNA, 23 bp, Artificial Sequence
  primer
ggaagtggggtgacagcctaaca

SEQ ID NO: 76 - DNA, 22 bp, Artificial Sequence
  primer
ggtgacagsctaacagwntttc

SEQ ID NO: 77 - DNA, 19 bp, Artificial Sequence
  primer
gcaccccagtggacgaatt

SEQ ID NO: 78 - DNA, 23 bp, Artificial Sequence
  primer
tcagcctcttggccttagtgtag

(19) United States
(12) Patent Application Publication
(10) Pub. No.: US 2005/0014158 A1
(43) Pub. Date: Jan. 20, 2005

(54) Therapeutic methods for reducing fat deposition and treating associated conditions

(76) Inventors: Gail Isabel Reid Adam, Knivsta (SE); Maria L. Langdown, San Diego, CA (US); Mikhail F. Denissenko, Poway, CA (US); Edward A. Dennis, La Jolla, CA (US); Charles Cantor, Del Mar, CA (US); Byron Rubin, Honeoye Falls, NY (US)

Correspondence Address: MORRISON & FOERSTER LLP, 3811 VALLEY CENTRE DRIVE, SUITE 500, SAN DIEGO, CA, 92130-2332, US

(21) Appl. No.: 10/607,806

(22) Filed: Jun. 27, 2003

Related U.S. Application Data
(60) Provisional application No. 60/392,362, filed on Jun. 27, 2002.

(51) Int. Cl. C12Q 1/68

(52) U.S. Cl. 435/6

(57) ABSTRACT

Provided herein are methods for prognosing and diagnosing fat deposition and related disorders (e.g., obesity and non-insulin diabetes dependent mellitus (NIDDM)) in a subject, reagents and kits for carrying out the methods, methods for identifying candidate therapeutics for reducing fat deposition and related disorders, and therapeutic methods for reducing fat deposition or treating fat deposition related disorders in a subject. These embodiments are based in part upon an analysis of polymorphic variations of the nucleic acid set forth in SEQ ID NO:1.

### CROSS-REFERENCE TO RELATED APPLICATIONS

This application claims the benefit of U.S. patent application No. 60/392,362 filed 27 Jun. 2002. The contents of that application is incorporated herein by reference.

### FIELD OF THE INVENTION

The invention relates to methods for identifying and using therapeutic agents for reducing fat deposition and treating associated conditions, including diabetes, in subjects. The therapeutic agents target a phospholipase associated with fat deposition.

### BACKGROUND

Individuals who are obsese have excess body fat compared to set standards. Obesity can be determined by several methods including body mass index (BMI) measurements, weight-for height charts, and body fat measurements determined by skinfold thickness and bioelectrical impedance. Obesity affects 58 million people across the United States, which represents approximately one-quarter to one-third of the adult population, and its prevalence is increasing to epidemic proportions in the United States and in other industrialized nations.

Recognized since 1985 as a chronic disease, obesity-related medical conditions contribute to approximately 300,000 deaths each year, second only to smoking as a cause of preventable death. (JAMA, 276: 1907-1915 (1996)). Obesity has been established as a major risk factor for type II diabetes melitus, hypertension, cardiovascular disease and some cancers in both men and women (JAMA, 282: 1523-1529 (1999)). Other comorbid conditions include sleep apnea, osteoarthritis, infertility, idiopathic intracranial hypertension, lower extremity venous stasis disease, gastro-esophageal reflux and urinary stress incontinence.

The total cost attributable to obesity amounted to $99.2 billion in 1995. Approximately $51.65 billion of those dollars were direct medical costs. The cost of obesity to U.S. business in 1994 was estimated to total $12.7 billion, and health-related economic costs of obesity to businesses in the United States is substantial, representing approximately 5% of total medical care costs. (American Journal of Health Promotion, 13 (2): 120-127 (1998)). It was found that as BMI increases, so do the number of sick days, medical claims and health care costs and that the mean annual health care costs for the BMI “at risk” population was $2,274 versus $1,499 for the “not at risk” group.

An accumulation of adipose tissue on the trunk and around the waist, known as central fat, also confers an increased risk of type II diabetes and cardiovascular disease (Lundgren et al., Int. J. Obes., 13(4): 413-23 (1989); Ohlson et al., Diabetes, 34(10): 1055-8 (1985)). In addition, central obesity has been implicated in a condition known as the metabolic syndrome (or syndrome X), which is associated with increased risk of cardiovascular disease, vascular dementia, and diabetes. The metabolic syndrom is a descriptive term for the coexistence of all of the following or differing combinations of central fat, hypertension, glucose intolerance, dyslipidemia (elevated triglycerides and low HDL cholesterol), and impaired insulin stimulated glucose uptake (“insulin resistance”). Prevalence of central fat and its relationship to general obesity differs between ethnic groups and gender (McKeigue et al., Diabetologia, 35(8): 785-91 (1992); McKeigue et al., Lancet, 337(8738): 382-6 (1991)). A majority of male subjects having high central fat are also obese in terms of BMI, and obese subjects often have a central distribution of fat, which suggests an overlap between these two conditions. While this relationship is not as strongly correlated in women, central fat increases after menopause.

Current anti-obesity therapeutics (e.g., Phentermine, Sibutramine, and Orlistat) are largely ineffective and there is an urgent need to define the etiology of this disease and initiate rational, mechanism-based drug development. Mouse QTL and human studies have postulated that the 12q22 to q23 region, and specifically the insulin-like growth factor 1 (IGF-1) gene in that region, play a role in body weight regulation and viseral fat deposition (Collins, A. C. et al., Mamm. Genome, 4: 454-458 (1993); Sun, G. et al., Int. J. Obes., 23: 929-935 (1999); Keightley, P. D. et al., Genetics, 142: 227-235 (1996). Also, other studies have linked obesity with certain portions of the human genome (Perusse, L. et al., Obesity Research, 9: 135-169(2001); Chagnon, Y. C. et al., Obesity Research, 8: 89-117 (2000)). Specifically, the CD36L gene on chromosome 12 was implicated in plasma lipid levels and with BMI (Acton, S. et al., Arterioscler. Thromb. Vasc. Biol., 19: 1734-1743 (1999)), the 12q24 chromosomal region was postulated as playing a role in obesity in a Quebec Family Study (Perusse, L. et al., Diabetes, 50: 614-621 (2001)), and it was reported that certain polymorphic loci on chromosome four are associated with obesity (Stone et al., American J. Human Genetics, “A major predisposition locus for severe obesity at 4p15-p14,” June 2002).

### SUMMARY

It has been discovered that polymorphic variations in or near a nucleotide sequence encoding a phospholipase A2 polypeptide known as PLA2G1B, which is located on chromosome twelve, are associated with central fat deposition. In addition, it was discovered that a polymorphic variation in the same nucleotide sequence was associated with type II diabetes (non-insulin dependent diabetes mellitus, or NIDDM) in subjects. Thus, PLA2G1B has been identified as a target for reducing fat deposition and treating associated conditions, including diabetes. Hence, featured herein are methods for identifying candidate therapeutic molecules that reduce fat deposition and treat related disorders, as well as methods of reducing fat deposition and treating related disorders in a subject by administering a therapeutic molecule.

### BRIEF DESCRIPTION OF THE DRAWINGS

FIGS. 1A to 1D depict the PLA2G1B nucleotide sequence reported as SEQ ID NO:1. The following nucleotide representations are used throughout: “A” or “a” is adenosine, adenine, or adenylic acid; “C” or “c” is cytidine, cytosine, or cytidylic acid; “G” or “g” is guanosine, guanine, or guaylic acid; “T” or “t” is thymidine, thymine, or thymidylic acid; and “I” or “i” is inosine, hypoxanthine, or inosinic acid. Exons are indicated in italicized lower case type, introns are depicted in normal text lower case type, and polymorphic sites are depicted in bold upper case type. SNPs are designated by the following convention: “R” represents A or G, “M” represents A or C; “W” represents A or T; “Y” represents C or T; “S” represents C or G; “K” represents G or T; “V” represents A, C or G; “H” represents A, C, or T; “D” represents A, G, or T; “B” represents C, G, or T; and “N” represents A, G, C, or T.

FIG. 2 shows a polypeptide sequence encoded by the nucleic acid of SEQ ID NO: 1.

FIGS. 3A and 3C depict tissue expression profiles for PLA2G1B and FIGS. 3B and 3D show expanded profiles of FIGS. 3A and 3C, respectively.

FIGS. 4A-4L show differential gene expression of PLA2G1B in metabolically-linked tissues, such as liver, fat pads, skeletal muscle, hypothalamus, pancreas, and stomach tissues from were analyzed following normal feeding or overnight fasting conditions. Studies were typically performed on group A (healthy), B (insulin resistant) and C animals (Diabetic/Obese), as group D animals (Diabetic/Obese) developed decompensated diabetes when their pancreas failed, leading to rapid death. In addition, the Figures contain data relating to blood glucose, plasma insulin, body weight, and body fat from the animals as compared to gene expression using t-test analysis. FIG. 4A shows PLA2G1B expression in the hypothalamus in group C fasted animals as compared to group A fasted animals and group B fasted animals. FIG. 4B shows hypothalamus PLA2G1B expression in group A animals that were fed normally versus fasted group A animals. FIG. 4C shows hypothalamus PLA2G1B expression in fasted animals versus body weight. FIG. 4D shows hypothalamus PLA2G1B expression in fasted animals versus plasma insulin levels. FIG. 4E shows expression in A fasted animals as compared to C fasted and B fasted animals. FIG. 4F shows expression in A fed group versus C fed group. FIGS. 4G, 4H and 4I show gene expression in fasted animals versus body weight, insulin and glucose. FIG. 4J shows liver PLA2G1B expression in fed animals versus body weight (p=0.013). FIG. 4K shows pancreatic PLA2G1B expression in control versus energy-restricted groups. FIG. 4L shows PLA2G1B expression in the fasted animals versus the fed animals.

FIG. 5A shows a nucleotide sequence alignment for human PLA2G1B and related sequences from mouse, rat, and P. obesus (sand rat). FIG. 5B shows an amino acid sequence alignment between human PLA2G1B and related sequences from mouse, rat, and P. obesus. The human PLA2G1B amino acid sequence in FIG. 5B has 148 amino acids and the mouse, rat, and P. obesus sequences have 146 amino acids. The human PLA2G1B amino acid sequence is 78% identical to the mouse sequence, 76% identical to the rat sequence, and 76% identical to the P. obesus sequence. The mouse sequence is 88% identical to the rat sequence and 77% identical to the P. obesus sequence, and the rat sequence is 80% identical to the P. obesus sequence.

### DETAILED DESCRIPTION

It has been discovered that polymorphic variants in or near a gene on chromosome 12 encoding a phospholipase are associated with fat deposition in the abdomen and trunk region of subjects. Individuals having increased fat deposition in this area are at risk of developing metabolic conditions (e.g., diabetes and obesity) and cardiovascular conditions (e.g., hypertension). Thus, methods for detecting genetic determinants for fat deposition can lead to early diagnosis of a predisposition to these conditions (e.g., hyperinsulinaemia, hypertension, glucose intolerance (that is, IGT or diabetes), dyslipidemia, hypercoagulability and microalbuminuria) and early prescription of preventative measures. Thus, associating PLA2G1B with fat deposition has provided a new target for screening molecules useful for treatments that reduce fat deposition. PLA2G1B is also a target for screening molecules useful for treating disorders associated with fat deposition, which include metabolic disorders (e.g., diabetes and obesity) and cardiovascular disorders (e.g., hypertension).

Central Fat Deposition and Associated Conditions

Many individuals considered as having increased central fat deposition are also considered obese according to BMI, weight-for-height charts, or body fat measurements. Obesity is generally understood as a condition where fat content in an individual is above a predetermined level. For example, the National Institute of Diabetes and Digestive and Kidney Diseases (NIDDK) of the National Institutes of Health (NIH, see http address www.nih.gov/health/nutrit/pubs/statobes.htm) define individuals having a body mass index (BMI) of 25 to 29.9 kg/m² as being overweight and individuals having a BMI of 30 kg/M² or greater as being obese.

Increased central fat levels also have been linked to the metabolic syndrome, which includes the coexistence or one or more life threatening medical conditions such as metabolic conditions (e.g., diabetes and obesity) and cardiovascular conditions (e.g., myocardial infarction and hypertension). For example, cardiovascular mortality was assessed in 3,606 subjects from the Botnia study (a large-scale study of type 2 diabetes begun in Finland in 1990) with a median follow-up of 6.9 years. In women and men, respectively, the metabolic syndrome was recorded in 10 and 15% of subjects with normal glucose tolerance, 42 and 64% of those with IFG/IGT, and 78 and 84% of those with type 2 diabetes. The risk for coronary heart disease and stroke was increased threefold in subjects with the syndrome, and cardiovascular mortality was markedly increased (12.0% in subjects with the syndrome versus 2.2% in those without; P<0.001) (Zimmet, et al. (2001) Nature 414: 782-787). Thus, determining a predisposition to fat deposition, and specifically central fat deposition, is useful for determining whether a person should be considered for being placed on a preventative regimen for reducing fat, thereby reducing the probability that the person develops one or more conditions linked to fat deposition.

The term “fat deposition” as used herein refers to fat content in an individual as well as processes in which fat is deposited in certain locations of an individual. The term “central fat deposition” as used herein refers to fat around the trunk and waist of an individual that is above a predetermined level or average in a population. The central region may be defined as the region extending from the superior surface of the second lumbar vertebra extending inferiorly to the inferior surface of the fourth lumbar vertebra and laterally to the inner aspect of the ribcage. Fat deposition can be measured as a quantity at one time point or a quantity over a series of time points, for example, and fat deposition can be quantified or estimated using a number of procedures described hereafter. Fat is composed of adipose cells deposited below the skin (i.e., subcutaneous adipose cells) and/or deeper within an individual's body (i.e., visceral adipose cells). Adipose cells are often connective tissue cells specialized for synthesis and storage of fat. Such cells often contain globules of triglycerides where the nucleus is generally displaced to one side of the globule and the cytoplasm is visualized as a thin line around the fat droplet. Provided herein are methods for detecting predisposition to overall adipose cell deposition in a subject (i.e., includes subcutaneous adipose cells and visceral adipose cells), as well as methods for distinguishing between a predisposition to subcutaneous adipose cell deposition and a predisposition to visceral adipose cell deposition.

Fat deposition may be quantified in a number of manners (see, e.g., Wajchenberg, Endocrine Rev. 21(6): 697-738 (2000)). For example, caliper measurements of skinfold thickness in defined areas of the body have been utilized to differ between different kinds of regional fat (Nordhamn, et al., Int. J. Obes. Relat. Metab. Disord. 24(5): 652-7 (2000)). Waist and hip measurements using tape measures are commonly utilized indices of central fat (Lundgren et al., Int. J. Obes., 13(4): 413-23 (1989); Ohlson et al., Diabetes 34(10): 1055-8 (1985)), and sagittal abdominal diameter is measured by some researchers for quantifying central fat. Also, computed tomography and X-ray based methods have been utilized to quantify central fat content. Dual x-ray absorbtiometry (DEXA) is relatively fast and inexpensive and yields reliable estimations of body composition (fat massaean mass/bone) with reproducibility. DEXA measurements and waist and hip measurements were utilized for quantifying central fat in Example 1. Magnetic resonance imaging (MRI) and computed tomography procedures can be used to distinguish between visceral fat deposition and subcutaneous fat deposition (see e.g. Wajchenberg, supra).

Thus, fat deposition can be expressed in terms of any units used for quantifying fat content. Fat deposition can be expressed in terms of total fat content in an individual or region of an individual (grams or percentage of total weight of an individual), visceral fat content in an individual or region of an individual (grams, percentage of total weight of an individual, or percentage of total fat in an individual), and subcutaneous fat content in an individual or region of an individual (grams, percentage of total weight of an individual, or percentage of total fat in an individual). Each of these expressions of fat deposition can be measured or quantified at a single point in time or over two or more points in time.

Fat deposition also can be expressed in terms of “increased fat deposition” (also referred to as “higher fat deposition” and “at increased risk for fat deposition”), which is relative to average fat deposition in a population. In a distribution of fat deposition across a population (expressed in any of the units of measure described herein), individuals having increased fat deposition are sometimes represented in the upper 40% or upper 30% of the population, often in the upper 25%, upper 20%, upper 15%, and upper 10% of the population, and sometimes in the upper 5% of the population. Also, individuals having increased fat deposition can be characterized as having waist/hip ratios of 1.01 or more for males and 0.91 or more for females. In addition, men or women having a BMI between 25 and 30 or between about 1335 and about 2050 grams of central fat are typically considered overweight, and individuals having a BMI over 30 or over about 2050 grams of central fat are normally considered obese (e.g., grams of central fat can be determined by DEXA, as described above). Also, “leanness” or “decreased fat deposition” (also referred to as “lower fat deposition” and “at decreased risk for fat deposition”) are terms that refer to fat deposition and are also relative to average fat deposition in a population. In a distribution of fat deposition across a population, lean individuals are sometimes represented in the lower 40% or lower 30% of the population, often in the lower 25%, lower 20%, lower 15%, and lower 10% of the population, and sometimes in the lower 5% of the population. Also, lean individuals can be characterized as having waist/hip ratios of 1.00 or less for males and 0.90 or less for females. In addition, men or women having a BMI of 24 or less or less than about 1334 grams of central fat are normally considered lean.

The term “metabolic condition” as used herein refers to a disease, disorder, or state involving increased or decreased metabolites relative to a population average. Examples of metabolic disorders include but are not limited to diabetes, obesity, anorexia nervosa, cachexia, and lipid disorders.

The term “NIDDM” as used herein refers to non-insulin-dependent diabetes mellitus or Type 2 diabetes (the two terms are used interchangeably throughout this document). NIDDM refers to an insulin-related disorder in which there is a relative disparity between endogenous insulin production and insulin requirements, leading to elevated hepatic glucose production, elevated blood glucose levels, inappropriate insulin secretion, and peripheral insulin resistance.

The term “cardiovascular condition” as used herein refers to a disease, disorder, or state involving the cardiovascular system, e.g., the heart, the blood vessels, and/or the blood. A cardiovascular disorder can be caused by an imbalance in arterial pressure, a malfunction of the heart, or an occlusion of a blood vessel (e.g., by a thrombus). Other examples of cardiovascular disorders include but are not limited to hypertension, atherosclerosis, coronary artery spasm, coronary artery disease, arrhythmias, heart failure, including but not limited to, cardiac hypertrophy, left-sided heart failure, and right-sided heart failure; ischemic heart disease, including but not limited to angina pectoris, myocardial infarction, chronic ischemic heart disease, and sudden cardiac death; hypertensive heart disease, including but not limited to, systemic (left-sided) hypertensive heart disease and pulmonary (right-sided) hypertensive heart disease; valvular heart disease, including but not limited to, valvular degeneration caused by calcification, such as calcification of a congenitally bicuspid aortic valve, and mitral annular calcification, and myxomatous degeneration of the mitral valve (mitral valve prolapse), rheumatic fever and rheumatic heart disease, infective endocarditis, and noninfected vegetations, such as nonbacterial thrombotic endocarditis and endocarditis of systemic lupus erythematosus (Libman-Sacks disease), carcinoid heart disease, and complications of artificial valves; myocardial disease, including but not limited to dilated cardiomyopathy, hypertrophic cardiomyopathy, restrictive cardiomyopathy, and myocarditis; pericardial disease, including but not limited to, pericardial effusion and hemopericardium and pericarditis, including acute pericarditis and healed pericarditis, and rheumatoid heart disease; neoplastic heart disease, including but not limited to, primary cardiac tumors, such as myxoma, lipoma, papillary fibroblastoma, rhabdomyoma, and sarcoma, and cardiac effects of noncardiac neoplasms; congenital heart disease, including but not limited to, left-to-right shunts (late cyanosis, such as atrial septal defect, ventricular septal defect, patent ductus arteriosis, and atrioventricular septal defect, right-to-left shunts), early cyanosis (e.g., tetralogy of fallot, transposition of great arteries, truncus arteriosis, tricuspid atresia, and total anomalous pulmonary venous connection), obstructive congenital anomalies (e.g., coarctation of aorta, pulmonary stenosis and atresia, and aortic stenosis and atresia), disorders involving cardiac transplantation, and congestive heart failure.

Polymorphic Variants Associated with Fat Deposition and Related Conditions

A genetic analysis provided herein linked fat deposition with polymorphic variants of a nucleotide sequence located on chromosome twelve that encodes a phospholipase A2 polypeptide designated PLA2G1B. An additional genetic analysis provided herein linked NIDDM with a polymorphic variant of a nucleotide sequence located on chromosome twelve that encodes a phospholipase A2, group IB designated PLA2G1B. As used herein, the term “polymorphic site” refers to a region in a nucleic acid at which two or more alternative nucleotide sequences are observed in a significant number of nucleic acid samples from a population of individuals. A polymorphic site may be a nucleotide sequence of two or more nucleotides, an inserted nucleotide or nucleotide sequence, a deleted nucleotide or nucleotide sequence, or a microsatellite, for example. A polymorphic site that is two or more nucleotides in length may be 4, 5, 6, 7, 8, 9, 10, 11, 12, 13, 14, 15 or more, 20 or more, 30 or more, 50 or more, 75 or more, 100 or more, 500 or more, or about 1000 nucleotides in length, where all or some of the nucleotide sequences differ within the region. A polymorphic site is often one nucleotide in length, which is referred to herein as a “single nucleotide polymorphism” or a “SNP.”

Where there are two, three, or four alternative nucleotide sequences at a polymorphic site, each nucleotide sequence is referred to as a “polymorphic variant.” Where two polymorphic variants exist, for example, the polymorphic variant represented in a minority of samples from a population is sometimes referred to as a “minor allele” and the polymorphic variant that is more prevalently represented is sometimes referred to as a “major allele.” Many organisms possess a copy of each chromosome (e.g., humans), and those individuals who possess two major alleles or two minor alleles are often referred to as being “homozygous” with respect to the polymorphism and those individuals who possess one major allele and one minor allele are normally referred to as being “heterozygous” with respect to the polymorphism. Individuals who are homozygous with respect to one allele are sometimes predisposed to a different phenotype as compared to individuals who are heterozygous or homozygous with respect to another allele. As used herein, the term “phenotype” refers to a trait which can be compared between individuals, such as presence or absence of a condition, a visually observable difference in appearance between individuals, metabolic variations, physiological variations, variations in the function of biological molecules, and the like. Examples of phenotypes are fat deposition, obesity, and diabetes.

Researchers sometimes report a polymorphic variant in a database without determining whether the variant is represented in a significant fraction of a population. Because a subset of these reported polymorphismic variants are not represented in a statistically significant portion of the population, some of them are sequencing errors and/or not biologically relevant. Thus, it is often not known whether a reported polymorphic variant is statistically significant or biologically relevant until the presence of the variant is detected in a population of individuals and the frequency of the variant is determined. Methods for detecting a polymorphic variant in a population are described herein, specifically in Example 2. A polymorphic variant is statistically significant and often biologically relevant if it is represented in 5% or more of a population, sometimes 10% or more, 15% or more, or 20% or more of a population, and often 25% or more, 30% or more, 35% or more, 40% or more, 45% or more, or 50% or more of a population.

A polymorphic variant may be detected on either or both strands of a double-stranded nucleic acid. Also, a polymorphic variant may be located within an intron or exon of a gene or within a portion of a regulatory region such as a promoter, a 5′ untranslated region (UTR), a 3, UTR, and in DNA (e.g., genomic DNA (GDNA) and complementary DNA (cDNA)), RNA (e.g., mRNA, tRNA, and rRNA), or a polypeptide. Polymorphic variations may or may not result in detectable differences in gene expression, polypeptide structure, or polypeptide function.

In the genetic analysis that associated polymorphic variations in PLA2G1B with fat deposition, samples from individuals in a population of twin pairs were genotyped, although other populations could be subjected to analysis. The term “genotyped” as used herein refers to a process for determining a genotype of one or more individuals, where a “genotype” is a representation of polymorphic variants in a population. Fat deposition was quantified in the central region of individuals in the study group, and SNPs were identified at positions 7328 and 9182 in the PLA2G1B nucleotide sequence represented by SEQ ID NO:1. It was determined that 84% of the individuals tested in the genetic analysis had a guanine at position 7328 and 16% of the individuals had an adenine at this position. At position 9182, 85% of the individuals had a thymine and 15% of the individuals had a guanine. It was determined that a guanine at position 7328 or a thymine at position 9182 were individually associated with central fat deposition, and the presence of an adenine at position 7328 or a guanine at position 9182 were individually associated with leanness.

In the genetic analysis that associated polymorphic variations in PLA2G1B with NIDDM, samples from individuals in a population of with NIDDM and without NIDDM were genotyped. A SNP was identified at position 7256 in the PLA2G1B nucleotide sequence represented by SEQ ID NO:1. It was determined that 93% of female controls tested in the genetic analysis had a thymine at position 7256 and 7% of the individuals had a cytosine at this position, while 92% of female cases tested in the genetic analysis had a thymine at position 7256 and 8% of the individuals had a cytosine at this position. It was also determined that 95% of male controls tested in the genetic analysis had a thymine at position 7256 and 5% of the individuals had a cytosine at this position, while 90% of male cases tested in the genetic analysis had a thymine at position 7256 and 10% of the individuals had a cytosine at this position. It was determined that a cytosine at position 7256 was individually associated with NIDDM, and the presence of a thymine at position 7256 was individually associated with not having NIDDM.

Furthermore, a genotype or polymorphic variant may be expressed in terms of a “haplotype,” which as used herein refers to two or more polymorphic variants occurring within genomic DNA in a group of individuals within a population. For example, two SNPs may exist within a gene where each SNP position includes a cytosine variation and an adenine variation. Certain individuals in a population may carry one allele (heterozygous) or two alleles (homozygous) having the gene with a cytosine at each SNP position. As the two cytosines corresponding to each SNP in the gene travel together on one or both alleles in these individuals, the individuals can be characterized as having a cytosine/cytosine haplotype with respect to the two SNPs in the gene.

Also, the genetic analysis identified haplotypes associated with lower risk of fat deposition. In particular, presence of a haplotype represented by TTAG or GTAG at positions 4050, 7256, 7328, and 9182, respectively, in the PLA2G1B sequence represented by SEQ ID NO:1 were associated with leanness. As used herein, a “haplotype” refers to a combination of polymorphic variations in a defined region within a genetic locus on one of the chromosomes in a chromosome pair.

Additional Polymorphic Variants Associated with Fat Deposition and Related Disorders

Also provided is a method for identifying polymorphic variants proximal to an incident, founder polymorphic variant associated with fat deposition, obesity and NIDDM. Thus, featured herein are methods for identifying a polymorphic variation associated with fat deposition or NIDDM that is proximal to an incident polymorphic variation associated with fat deposition or NIDDM, which comprises identifying a polymorphic variant proximal to the incident polymorphic variant associated with fat deposition of NIDDM, where the incident polymorphic variant is in a PLA2G1B nucleotide sequence. The PLA2G1B nucleotide sequence often comprises a polynucleotide sequence selected from the group consisting of (a) a polynucleotide sequence set forth in SEQ ID NO: 1; (b) a polynucleotide sequence that encodes a polypeptide having an amino acid sequence encoded by a nucleotide sequence set forth as SEQ ID NO: 1; or (c) a polynucleotide sequence that encodes a polypeptide having an amino acid sequence that is 90% identical to an amino acid sequence encoded by a nucleotide sequence set forth in SEQ ID NO: 1 or a polynucleotide sequence 90% identical to the polynucleotide sequence of SEQ ID NO:1. The presence or absence of an association of the proximal polymorphic variant with fat deposition or NIDDM then is determined using a known association method, such as a method described in the Examples hereafter. In an embodiment, the incident polymorphic variant is at position 7256, 7328, or 9182 of SEQ ID NO: 1. In another embodiment, the proximal polymorphic variant identified sometimes is a publicly disclosed polymorphic variant, which for example, sometimes is published in a publicly available database. In other embodiments, the polymorphic variant identified is not publicly disclosed and is discovered using a known method, including, but not limited to, sequencing a region surrounding the incident polymorphic variant in a group of nucleic samples. Thus, multiple polymorphic variants proximal to an incident polymorphic variant are associated with fat deposition and NIDDM using this method.

The proximal polymorphic variant often is identified in a region surrounding the incident polymorphic variant. In certain embodiments, this surrounding region is about 50 kb flanking the first polymorphic variant (e.g. about 50 kb 5′ of the first polymorphic variant and about 50 kb 3′ of the first polymorphic variant), and the region sometimes is composed of shorter flanking sequences, such as flanking sequences of about 40 kb, about 30 kb, about 25 kb, about 20 kb, about 15 kb, about 10 kb, about 7 kb, about 5 kb, or about 2 kb 5′ and 3′ of the incident polymorphic variant. In other embodiments, the region is composed of longer flanking sequences, such as flanking sequences of about 55 kb, about 60 kb, about 65 kb, about 70 kb, about 75 kb, about 80 kb, about 85 kb, about 90 kb, about 95 kb, or about 100 kb 5′ and 3′ of the incident polymorphic variant.

In certain embodiments, polymorphic variants associated with fat deposition or NIDDM are identified iteratively. For example, a first proximal polymorphic variant is associated with fat deposition using the methods described above and then another polymorphic variant proximal to the first proximal polymorphic variant is identified (e.g., publicly disclosed or discovered) and the presence or absence of an association of one or more other polymorphic variants proximal to the first proximal polymorphic variant with fat deposition or NIDDM is determined.

The methods described herein are useful for identifying or discovering additional polymorphic variants that may be used to further characterize a gene, region or loci associated with a condition, a disease (e.g., fat deposition or NIDDM), or a disorder. For example, allelotyping or genotyping data from the additional polymorphic variants may be used to identify a functional mutation or a region of linkage disequilibrium.

In certain embodiments, polymorphic variants identified or discovered within a region comprising the first polymorphic variant associated with fat deposition or NIDDM are genotyped using the genetic methods and sample selection techniques described herein, and it can be determined whether those polymorphic variants are in linkage disequilibrium with the first polymorphic variant. The size of the region in linkage disequilibrium with the first polymorphic variant also can be assessed using these genotyping methods. Thus, provided herein are methods for determining whether a polymorphic variant is in linkage disequilibrium with a first polymorphic variant associated with fat deposition or NIDDM, and such information can be used in prognosis methods described herein.

Isolated PLA2G1B Nucleic Acids and Variants Thereof

Featured herein are isolated PLA2G1B nucleic acids, which include the nucleic acid having the nucleotide sequence of SEQ ID NO:1, PLA2G1B nucleic acid variants, and substantially identical nucleic acids to the foregoing. Nucleotide sequences of the PLA2G1B nucleic acids are sometimes referred to herein as “PLA2G1B nucleotide sequences.” A “PLA2G1B nucleic acid variant” refers to one allele that may have different polymorphic variations as compared to another allele in another subject or the same subject. A polymorphic variation in the PLA2G1B nucleic acid variant may be represented on one or both strands in a double-stranded nucleic acid or on one chromosomal complement (heterozygous) or both chromosomal complements (homozygous)). A PLA2G1B nucleic acid may comprise one or more of the following polymorphic variations: a thymine or a cytosine at position 7256 of SEQ ID NO:1 in a strand, or an adenine or guanine in a complementary strand; an adenine or guanine at position 7328 of SEQ ID NO:1 in a strand, or a thymine or cytosine in a complementary strand; or a guanine or thymine at position 9182 of SEQ ID NO:1 in a strand, or a cytosine or adenine in a complementary strand; presence of GTGT, TTGT, TTAG, GCGT, or GTAG at positions 4050, 7256, 7328, and 9182 of SEQ ID NO:1, respectively, in a strand, or presence of CACA, AACA, AATC, CGCA, or CATC in a complementary strand.

As used herein, the term “nucleic acid” includes DNA molecules (e.g., a complementary DNA (cDNA) and genomic DNA (gDNA)) and RNA molecules (e.g., mRNA, rRNA, siRNA and tRNA) and analogs of DNA or RNA, for example, by use of nucleotide analogs. The nucleic acid molecule can be single-stranded and it is often double-stranded. The term “isolated or purified nucleic acid” refers to nucleic acids that are separated from other nucleic acids present in the natural source of the nucleic acid. For example, with regard to genomic DNA, the term “isolated” includes nucleic acids which are separated from the chromosome with which the genomic DNA is naturally associated. An “isolated” nucleic acid is often free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. As used herein, the term “PLA2G1B gene” refers to a nucleotide sequence that encodes a PLA2G1B polypeptide.

Also included herein are nucleic acid fragments. These fragments are typically a nucleotide sequence identical to a nucleotide sequence in SEQ ID NO:1, a nucleotide sequence substantially identical to a nucleotide sequence in SEQ ID NO:1, or a nucleotide sequence that is complementary to the foregoing. The nucleic acid fragment may be identical, substantially identical or homologous to a nucleotide sequence in an exon or an intron in SEQ ID NO:1 and may encode a domain or part of a domain of a PLA2G1B polypeptide. Sometimes, the fragment will comprises one or more of the polymorphic variations described herein as being associated with increased fat deposition or increased risk of developing NIDDM. The nucleic acid fragment is often 50, 100, or 200 or fewer base pairs in length, and is sometimes about 300, 400, 500, 600, 700, 800, 900, 1000, 1100, 1200, 1300, 1400, 1500, 2000, 3000, 4000, 5000, 10000, or 12000 base pairs in length. A nucleic acid fragment that is complementary to a nucleotide sequence identical or substantially identical to the nucleotide sequence of SEQ ID NO:1 and hybridizes to such a nucleotide sequence under stringent conditions is often referred to as a “probe.” Nucleic acid fragments often include one or more polymorphic sites, or sometimes have an end that is adjacent to a polymorphic site as described hereafter.

An example of a nucleic acid fragment is an oligonucleotide. As used herein, the term “oligonucleotide” refers to a nucleic acid comprising about 8 to about 50 covalently linked nucleotides, often comprising from about 8 to about 35 nucleotides, and more often from about 10 to about 25 nucleotides. The backbone and nucleotides within an oligonucleotide may be the same as those of naturally occurring nucleic acids, or analogs or derivatives of naturally occurring nucleic acids, provided that oligonucleotides having such analogs or derivatives retain the ability to hybridize specifically to a nucleic acid comprising a targeted polymorphism. Oligonucleotides described herein may be used as hybridization probes or as components of diagnostic assays, for example, as described herein.

Oligonucleotides are typically synthesized using standard methods and equipment, such as the ABI™3900 High Throughput DNA Synthesizer and the EXPEDITE™ 8909 Nucleic Acid Synthesizer, both of which are available from Applied Biosystems (Foster City, Calif.). Analogs and derivatives are exemplified in U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; WO 00/56746; WO 01/14398, and related publications. Methods for synthesizing oligonucleotides comprising such analogs or derivatives are disclosed, for example, in the patent publications cited above and in U.S. Pat. Nos. 5,614,622; 5,739,314; 5,955,599; 5,962,674; 6,117,992; in WO 00/75372; and in related publications.

Oligonucleotides may also be linked to a second moiety. The second moiety may be an additional nucleotide sequence such as a tail sequence (e.g., a polyadenosine tail), an adaptor sequence (e.g., phage M13 universal tail sequence), and others. Alternatively, the second moiety may be a non-nucleotide moiety such as a moiety which facilitates linkage to a solid support or a label to facilitate detection of the oligonucleotide. Such labels include, without limitation, a radioactive label, a fluorescent label, a chemiluminescent label, a paramagnetic label, and the like. The second moiety may be attached to any position of the oligonucleotide, provided the oligonucleotide can hybridize to the nucleic acid comprising the polymorphism.

Uses for Nucleic Acid Sequence

Nucleic acid coding sequences depicted in SEQ ID NO: 1 may be used for diagnostic purposes for detection and control of polypeptide expression. Also, included herein are oligonucleotide sequences such as antisense RNA, small-interfering RNA (siRNA) and DNA molecules and ribozymes that function to inhibit translation of a polypeptide. Antisense techniques and RNA interference techniques are known in the art and are described herein.

Ribozymes are enzymatic RNA molecules capable of catalyzing the specific cleavage of RNA. The mechanism of ribozyme action involves sequence specific hybridization of the ribozyme molecule to complementary target RNA, followed by a endonucleolytic cleavage. Ribozymes may be engineered hammerhead motif ribozyme molecules that specifically and efficiently catalyze endonucleolytic cleavage of RNA sequences corresponding to or complementary to the nucleotide sequences set forth in SEQ ID NO: 1. Specific ribozyme cleavage sites within any potential RNA target are initially identified by scanning the target molecule for ribozyme cleavage sites which include the following sequences, GUA, GUU and GUC. Once identified, short RNA sequences of between fifteen (15) and twenty (20) ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridization with complementary oligonucleotides, using ribonuclease protection assays.

Antisense RNA and DNA molecules, siRNA and ribozymes may be prepared by any method known in the art for the synthesis of RNA molecules. These include techniques for chemically synthesizing oligodeoxyribonucleotides well known in the art such as solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

DNA encoding a polypeptide also may have a number of uses for the diagnosis of diseases, including fat deposition or NIDDM, resulting from aberrant expression of PLA21GB. For example, the nucleic acid sequence may be used in hybridization assays of biopsies or autopsies to diagnose abnormalities of expression or function (e.g., Southern or Northern blot analysis, in situ hybridization assays).

In addition, the expression of a polypeptide during embryonic development may also be determined using nucleic acid encoding the polypeptide. As addressed, infra, production of functionally impaired polypeptide is the cause of various disease states, including fat deposition or NIDDM. In situ hybridizations using polypeptide as a probe may be employed to predict problems related to obesity or NIDDM. Further, as indicated, infra, administration of human active polypeptide, recombinantly produced as described herein, may be used to treat disease states related to functionally impaired polypeptide. Alternatively, gene therapy approaches may be employed to remedy deficiencies of functional polypeptide or to replace or compete with dysfunctional polypeptide.

Expression Vectors, Host Cells, and Genetically Engineered Cells

Provided herein are nucleic acid vectors, often expression vectors, which contain a PLA2G1B nucleic acid. As used herein, the term “vector” refers to a nucleic acid molecule capable of transporting another nucleic acid to which it has been linked and can include a plasmid, cosmid, or viral vector. The vector can be capable of autonomous replication or it can integrate into a host DNA. Viral vectors may include replication defective retroviruses, adenoviruses and adeno-associated viruses for example.

A vector can include a PLA2G1B nucleic acid in a form suitable for expression of the nucleic acid in a host cell. The recombinant expression vector typically includes one or more regulatory sequences operatively linked to the nucleic acid sequence to be expressed. The term “regulatory sequence” includes promoters, enhancers and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of expression of polypeptide desired, and the like. Expression vectors can be introduced into host cells to produce PLA2G1B polypeptides, including fusion polypeptides, encoded by PLA2G1B nucleic acids.

Recombinant expression vectors can be designed for expression of PLA2G1B polypeptides in prokaryotic or eukaryotic cells. For example, PLA2G1B polypeptides can be expressed in E. coli, insect cells (e.g., using baculovirus expression vectors), yeast cells, or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of polypeptides in prokaryotes is most often carried out in E. coli with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors typically serve three purposes: 1) to increase expression of recombinant polypeptide; 2) to increase the solubility of the recombinant polypeptide; and 3) to aid in the purification of the recombinant polypeptide by acting as a ligand in affinity purification. Often, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin and enterokinase. Typical fusion expression vectors include pGEX (Pharmacia Biotech Inc; Smith, D. B. and Johnson, K. S., Gene 67: 31-40 (1988)), pMAL (New England Biolabs, Beverly, Mass.) and pRIT5 (Pharmacia, Piscataway, N.J.) which fuse glutathione S-transferase (GSI), maltose E binding polypeptide, or polypeptide A, respectively, to the target recombinant polypeptide.

Purified fusion polypeptides can be used in screening assays and to generate antibodies specific for PLA2G1B polypeptides. In a therapeutic embodiment, fusion polypeptide expressed in a retroviral expression vector is used to infect bone marrow cells that are subsequently transplanted into irradiated recipients. The pathology of the subject recipient is then examined after sufficient time has passed (e.g., six (6) weeks).

Expressing the polypeptide in host bacteria with an impaired capacity to proteolytically cleave the recombinant polypeptide is often used to maximize recombinant polypeptide expression (Gottesman, S., Gene Expression Technology: Methods in Enzymology, Academic Press, San Diego, Calif. 185: 119-128 (1990)). Another strategy is to alter the nucleotide sequence of the nucleic acid to be inserted into an expression vector so that the individual codons for each amino acid are those preferentially utilized in E. coli (Wada et al., Nucleic Acids Res. 20: 2111-2118 (1992)). Such alteration of nucleotide sequences can be carried out by standard DNA synthesis techniques.

When used in mammalian cells, the expression vector's control functions are often provided by viral regulatory elements. For example, commonly used promoters are derived from polyoma, Adenovirus 2, cytomegalovirus and Simian Virus 40. Recombinant mammalian expression vectors are often capable of directing expression of the nucleic acid in a particular cell type (e.g., tissue-specific regulatory elements are used to express the nucleic acid). Non-limiting examples of suitable tissue-specific promoters include an albumin promoter (liver-specific; Pinkert et al., Genes Dev. 1: 268-277 (1987)), lymphoid-specific promoters (Calame and Eaton, Adv. Immunol. 43: 235-275 (1988)), promoters of T cell receptors (Winoto and Baltimore, EMBO J. 8: 729-733 (1989)) promoters of immunoglobulins (Banerji et al., Cell 33: 729-740 (1983); Queen and Baltimore, Cell 33: 741-748 (1983)), neuron-specific promoters (e.g., the neurofilament promoter; Byrne and Ruddle, Proc. Natl. Acad. Sci. USA 86: 5473-5477 (1989)), pancreas-specific promoters (Edlund et al., Science 230: 912-916 (1985)), and mammary gland-specific promoters (e.g., milk whey promoter; U.S. Pat. No.4,873,316 and European Application Publication No. 264,166). Developmentally-regulated promoters are sometimes utilized, for example, the murine hox promoters (Kessel and Gruss, Science 249: 374-379 (1990)) and the α-fetopolypeptide promoter (Campes and Tilghman, Genes Dev. 3: 537-546 (1989)).

A PLA2G1B nucleic acid may also be cloned into an expression vector in an antisense orientation. Regulatory sequences (e.g., viral promoters and/or enhancers) operatively linked to a PLA2G1B nucleic acid cloned in the antisense orientation can be chosen for directing constitutive, tissue specific or cell type specific expression of antisense RNA in a variety of cell types. Antisense expression vectors can be in the form of a recombinant plasmid, phagemid or attenuated virus. For a discussion of the regulation of gene expression using antisense genes see Weintraub, H. et al., Antisense RNA as a molecular tool for genetic analysis, Reviews—Trends in Genetics, Vol. 1(1) (1986).

Also provided herein are host cells that include a PLA2G1B nucleic acid within a recombinant expression vector or PLA2G1B nucleic acid sequence fragments which allow it to homologously recombine into a specific site of the host cell genome. The terms “host cell” and “recombinant host cell” are used interchangeably herein. Such terms refer not only to the particular subject cell but rather also to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, a PLA2G1B polypeptide can be expressed in bacterial cells such as E. coli, insect cells, yeast or mammalian cells (such as Chinese hamster ovary cells (CHO) or COS cells). Other suitable host cells are known to those skilled in the art.

Vectors can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell provided herein can be used to produce (i.e., express) a PLA2G1B polypeptide. Accordingly, further provided are methods for producing a PLA2G1B polypeptide using the host cells of the invention. In one embodiment, the method includes culturing host cells into which a recombinant expression vector encoding a PLA2G1B polypeptide has been introduced in a suitable medium such that a PLA2G1B polypeptide is produced. In another embodiment, the method further includes isolating a PLA2G1B polypeptide from the medium or the host cell.

Also provided are cells or purified preparations of cells which include a PLA2G1B transgene, or which otherwise misexpress PLA2G1B polypeptide. Cell preparations can consist of human or non-human cells, e.g., rodent cells, e.g., mouse or rat cells, rabbit cells, or pig cells. In preferred embodiments, the cell or cells include a PLA2G1B transgene (e.g., a heterologous form of a PLA2G1B such as a human gene expressed in non-human cells). The PLA2G1B transgene can be misexpressed, e.g., overexpressed or underexpressed. In other preferred embodiments, the cell or cells include a gene which misexpress an endogenous PLA2G1B polypeptide (e.g., expression of a gene is disrupted, also known as a knockout). Such cells can serve as a model for studying disorders which are related to mutated or mis-expressed PLA2G1B alleles or for use in drug screening. Also provided are human cells (e.g., a hematopoietic stem cells) transformed with a PLA2G1B nucleic acid.

Also provided are cells or a purified preparation thereof (e.g., human cells) in which an endogenous PLA2G1B nucleic acid is under the control of a regulatory sequence that does not normally control the expression of the endogenous PLA2G1B gene. The expression characteristics of an endogenous gene within a cell (e.g., a cell line or microorganism) can be modified by inserting a heterologous DNA regulatory element into the genome of the cell such that the inserted regulatory element is operably linked to the endogenous PLA2G1B gene. For example, an endogenous PLA2G1B gene (e.g., a gene which is “transcriptionally silent,” not normally expressed, or expressed only at very low levels) may be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published on May 16, 1991.

Transgenic Animals

Non-human transgenic animals that express a heterologous PLA2G1B polypeptide (e.g., expressed from a PLA2G1B nucleic acid isolated from another organism) can be generated. Such animals are useful for studying the function and/or activity of a PLA2G1B polypeptide and for identifying and/or evaluating modulators of PLA2G1B nucleic acid and PLA2G1B polypeptide activity. As used herein, a “transgenic animal” is a non-human animal such as a mammal (e.g., a non-human primate such as chimpanzee, baboon, or macaque; an ungulate such as an equine, bovine, or caprine; or a rodent such as a rat, a mouse, or an Israeli sand rat), a bird (e.g., a chicken or a turkey), an amphibian (e.g., a frog, salamander, or newt), or an insect (e.g., drosophila melanogaster), in which one or more of the cells of the animal includes a PLA2G1B transgene. A transgene is exogenous DNA or a rearrangement (e.g., a deletion of endogenous chromosomal DNA) that is often integrated into or occurs in the genome of cells in a transgenic animal. A transgene can direct expression of an encoded gene product in one or more cell types or tissues of the transgenic animal, and other transgenes can reduce expression (e.g., a knockout). Thus, a transgenic animal can be one in which an endogenous PLA2G1B gene has been altered by homologous recombination between the endogenous gene and an exogenous DNA molecule introduced into a cell of the animal (e.g., an embryonic cell of the animal) prior to development of the animal.

Intronic sequences and polyadenylation signals can also be included in the transgene to increase expression efficiency of the transgene. One or more tissue-specific regulatory sequences can be operably linked to a PLA2G1B transgene to direct expression of a PLA2G1B polypeptide to particular cells. A transgenic founder animal can be identified based upon the presence of a PLA2G1B transgene in its genome and/or expression of PLA2G1B mRNA in tissues or cells of the animals. A transgenic founder animal can then be used to breed additional animals carrying the transgene. Moreover, transgenic animals carrying a transgene encoding a PLA2G1B polypeptide can further be bred to other transgenic animals carrying other transgenes.

PLA2G1B polypeptides can be expressed in transgenic animals or plants by introducing, for example, a nucleic acid encoding the polypeptide into the genome of an animal. In preferred embodiments the nucleic acid is placed under the control of a tissue specific promoter, e.g., a milk or egg specific promoter, and recovered from the milk or eggs produced by the animal. Also included is a population of cells from a transgenic animal.

PLA2G1B Polypeptides

Also featured herein are isolated PLA2G1B polypeptides, which include a polypeptide having the amino acid sequence of SEQ ID NO:2, PLA2G1B polypeptide variants, and substantially identical polypeptides thereof. A PLA2G1B polypeptide is a polypeptide encoded by a PLA2G1B nucleic acid, where one nucleic acid can encode one or more different polypeptides. An “isolated” or “purified” polypeptide or protein is substantially free of cellular material or other contaminating proteins from the cell or tissue source from which the protein is derived, or substantially free from chemical precursors or other chemicals when chemically synthesized. In one embodiment, the language “substantially free” means preparation of a PLA2G1B polypeptide or PLA2G1B polypeptide variant having less than about 30%, 20%, 10% and more preferably 5% (by dry weight), of non-PLA2G1B polypeptide (also referred to herein as a “contaminating protein”), or of chemical precursors or non-PLA2G1B chemicals. When the PLA2G1B polypeptide or a biologically active portion thereof is recombinantly produced, it is also preferably substantially free of culture medium, specifically, where culture medium represents less than about 20%, sometimes less than about 10%, and often less than about 5% of the volume of the polypeptide preparation. Isolated or purified PLA2G1B polypeptide preparations are sometimes 0.01 milligrams or more or 0.1 milligrams or more, and often 1.0 milligrams or more and 10 milligrams or more in dry weight.

Further included herein are PLA2G1B polypeptide fragments. The polypeptide fragment may be a domain or part of a domain of a PLA2G1B polypeptide. PLA2G1B domains include, but are not limited to, a phospholipase A2 domain at about amino acid positions 24 to 146 of SEQ ID NO:2. The polypeptide fragment may have increased, decreased or unexpected biological activity. The polypeptide fragment is often 50 or fewer, 100 or fewer, or 148 or fewer amino acids in length.

Substantially identical polypeptides may depart from the amino acid sequence of SEQ ID NO:2 in different manners. For example, conservative amino acid modifications may be introduced at one or more positions in the amino acid sequence of SEQ ID NO:2. A “conservative amino acid substitution” is one in which the amino acid is replaced by another amino acid having a similar structure and/or chemical function. Families of amino acid residues having similar structures and functions are well known. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Also, essential and non-essential amino acids may be replaced. A “non-essential” amino acid is one that can be altered without abolishing or substantially altering the biological function of a PLA2G1B polypeptide, whereas altering an “essential” amino acid abolishes or substantially alters the biological function of a PLA2G1B polypeptide. Amino acids that are conserved among phospholipase A2 polypeptides (e.g., P2X1, P2X2, P2X3, PLA2G1B, P2X5, P2X6, and P2X7) are typically essential amino acids.

Also, PLA2G1B polypeptides and polypeptide variants may exist as chimeric or fusion polypeptides. As used herein, a PLA2G1B “chimeric polypeptide” or “fusion polypeptide” includes a PLA2G1B polypeptide linked to a non-PLA2G1B polypeptide. A “non-PLA2G1B polypeptide” refers to a polypeptide having an amino acid sequence corresponding to a polypeptide which is not substantially identical to the PLA2G1B polypeptide, which includes, for example, a polypeptide that is different from the PLA2G1B polypeptide and derived from the same or a different organism. The PLA2G1B polypeptide in the fusion polypeptide can correspond to an entire or nearly entire PLA2G1B polypeptide or a fragment thereof. The non-PLA2G1B polypeptide can be fused to the N-terminus or C-terminus of the PLA2G1B polypeptide.

Fusion polypeptides can include a moiety having high affinity for a ligand. For example, the fusion polypeptide can be a GST-PLA2G1B fusion polypeptide in which the PLA2G1B sequences are fused to the C-terminus of the GST sequences, or a polyhistidine-PLA2G1B fusion polypeptide in which the PLA2G1B polypeptide is fused at the N— or C-terminus to a string of histidine residues. Such fusion polypeptides can facilitate purification of recombinant PLA2G1B. Expression vectors are commercially available that already encode a fusion moiety (e.g., a GST polypeptide), and a PLA2G1B nucleic acid can be cloned into an expression vector such that the fusion moiety is linked in-frame to the PLA2G1B polypeptide. Further, the fusion polypeptide can be a PLA2G1B polypeptide containing a heterologous signal sequence at its N-terminus. In certain host cells (e.g., mammalian host cells), expression, secretion, cellular internalization, and cellular localization of a PLA2G1B polypeptide can be increased through use of a heterologous signal sequence. Fusion polypeptides can also include all or a part of a serum polypeptide (e.g., an IgG constant region or human serum albumin).

PLA2G1B polypeptides can be incorporated into pharmaceutical compositions and administered to a subject in vivo. Administration of these PLA2G1B polypeptides can be used to affect the bioavailability of a PLA2G1B substrate and may effectively increase PLA2G1B biological activity in a cell. PLA2G1B fusion polypeptides may be useful therapeutically for the treatment of disorders caused by, for example, (i) aberrant modification or mutation of a gene encoding a PLA2G1B polypeptide; (ii) mis-regulation of the PLA2G1B gene; and (iii) aberrant post-translational modification of a PLA2G1B polypeptide. Also, PLA2G1B polypeptides can be used as immunogens to produce anti-PLA2G1B antibodies in a subject, to purify PLA2G1B ligands or binding partners, and in screening assays to identify molecules which inhibit or enhance the interaction of PLA2G1B with a PLA2G1B substrate.

In addition, polypeptides of the invention can be chemically synthesized using techniques known in the art (See, e.g., Creighton, 1983 Proteins. New York, N.Y.: W. H. Freeman and Company; and Hunkapiller et al., (1984) Nature July 12-18;310(5973):105-1 1). For example, a relative short fragment of the invention can be synthesized by use of a peptide synthesizer. Furthermore, if desired, nonclassical amino acids or chemical amino acid analogs can be introduced as a substitution or addition into the fragment sequence. Non-classical amino acids include, but are not limited to, to the D-isomers of the common amino acids, 2,4-diaminobutyric acid, a-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, omithine, norleucine, norvaline, hydroxyproline, sarcosine, citrulline, homocitrulline, cysteic acid, t-butylglycine, t-butylalanine, phenylglycine, cyclohexylalanine, b-alanine, fluoroamino acids, designer amino acids such as b-methyl amino acids, Ca-methyl amino acids, Na-methyl amino acids, and amino acid analogs in general. Furthermore, the amino acid can be D (dextrorotary) or L (levorotary).

The invention encompasses polypeptide fragments which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH4; acetylation, formylation, oxidation, reduction; metabolic synthesis in the presence of tunicamycin; etc.

Additional post-translational modifications encompassed by the invention include, for example, N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of procaryotic host cell expression. The polypeptide fragments may also be modified with a detectable label, such as an enzymatic, fluorescent, isotopic or affinity label to allow for detection and isolation of the polypeptide.

Also provided by the invention are chemically modified derivatives of the polypeptides of the invention that may provide additional advantages such as increased solubility, stability and circulating time of the polypeptide, or decreased immunogenicity. See U.S. Pat. No: 4,179,337. The chemical moieties for derivitization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

The polyethylene glycol molecules (or other chemical moieties) should be attached to the polypeptide with consideration of effects on functional or antigenic domains of the polypeptide. There are a number of attachment methods available to those skilled in the art, e.g., EP 0 401 384, herein incorporated by reference (coupling PEG to G-CSF), see also Malik et al. (1992) Exp Hematol. September;20(8):1028-35, reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues, glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

One may specifically desire proteins chemically modified at the N-terminus. Using polyethylene glycol as an illustration of the present composition, one may select from a variety of polyethylene glycol molecules (by molecular weight, branching, etc.), the proportion of polyethylene glycol molecules to protein (polypeptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus may be accomplished by reductive alkylation, which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

Substantially Identical PLA2G1B Nucleic Acids and Polypeptides

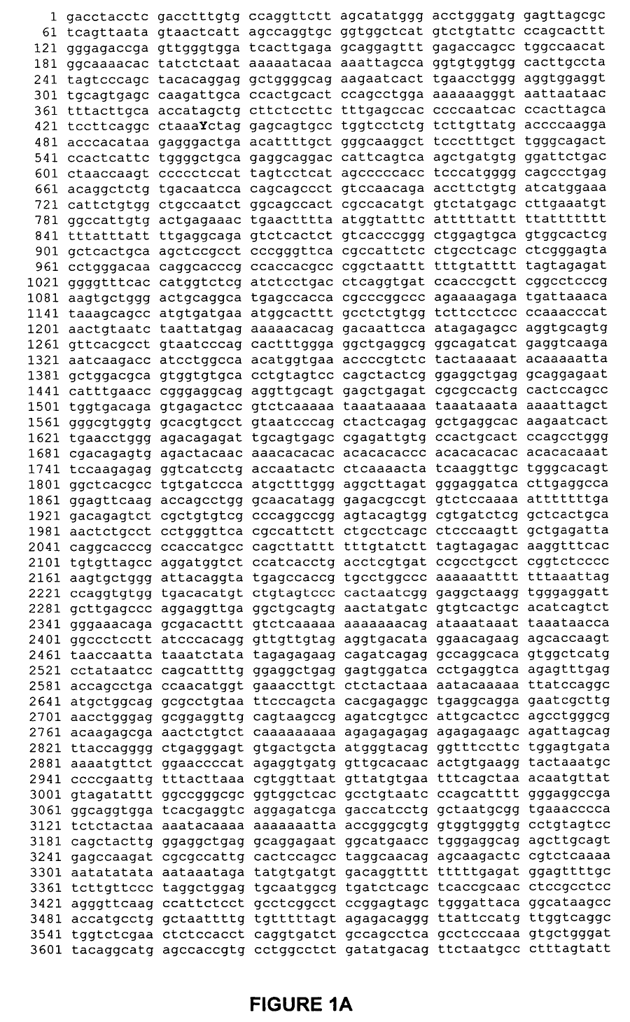

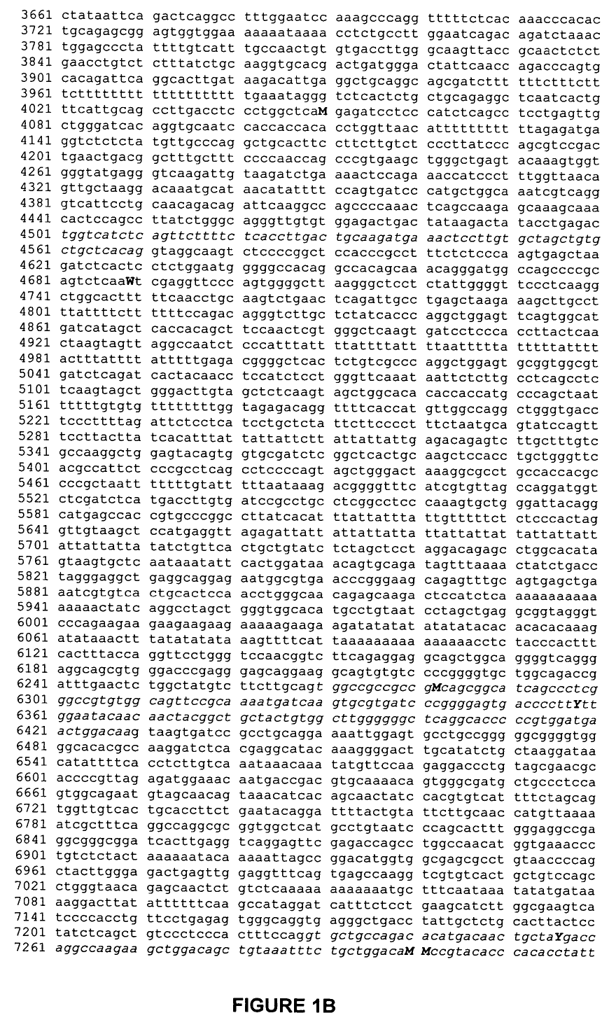

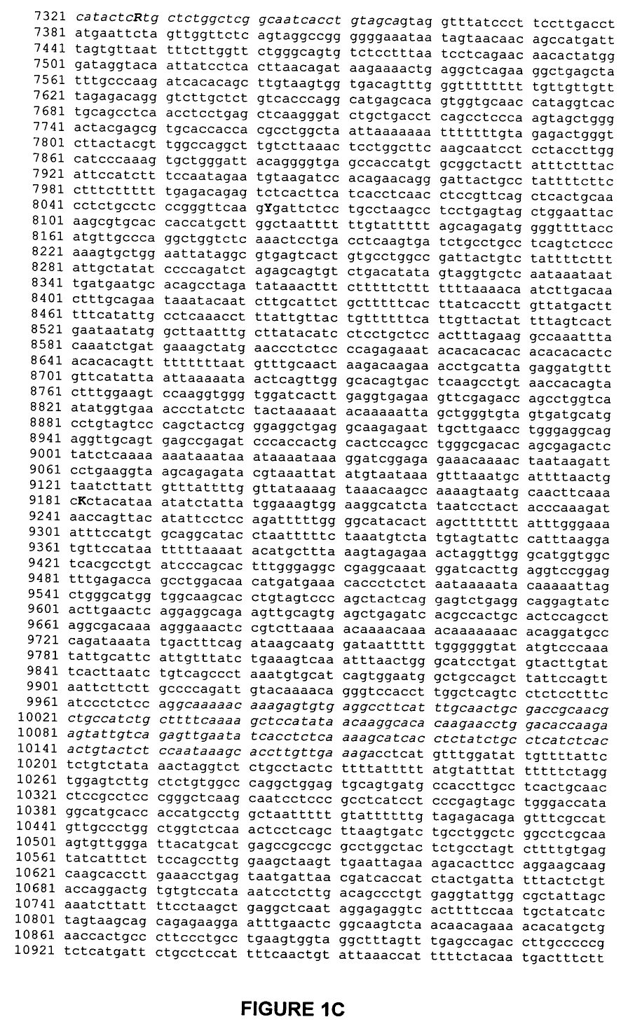

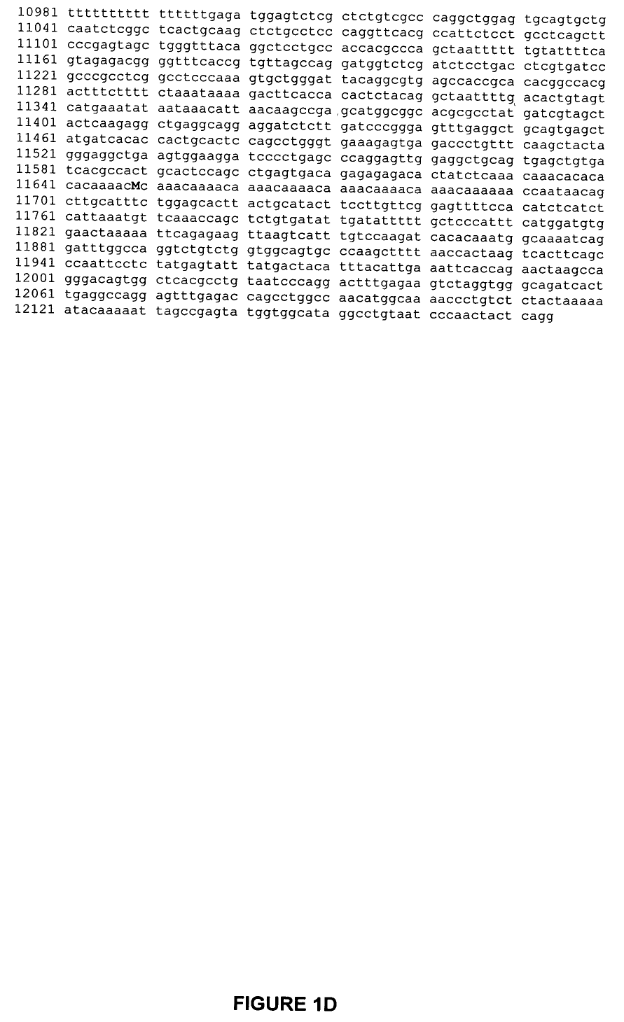

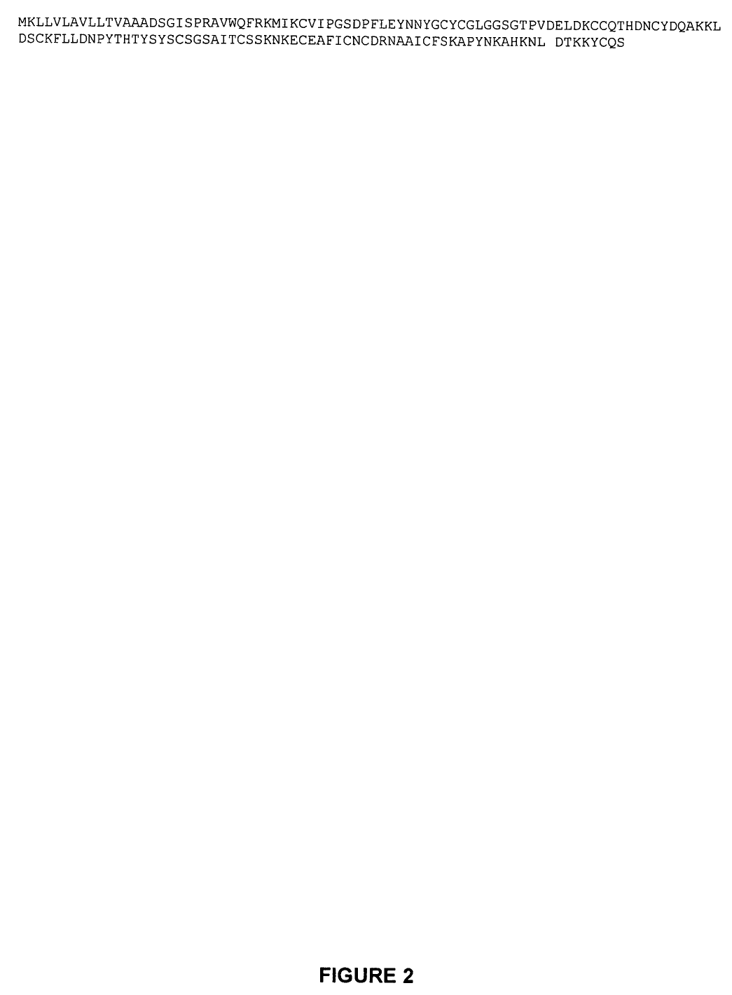

PLA2G1B nucleotide sequences and PLA2G1B polypeptide sequences that are substantially identical to the nucleotide sequence of SEQ ID NO:1 and the polypeptide sequence of SEQ ID NO:2. respectively, are included herein. The term “substantially identical” as used herein refers to two or more nucleic acids or polypeptides sharing one or more identical nucleotide sequences or polypeptide sequences, respectively. Included are nucleotide sequences or polypeptide sequences that are 55%, 60%, 65%, 70%, 75%, 80%, 85%, 90%, 95% (each often within a 1%, 2%, 3% or 4% variability) identical to the PLA2G1B nucleotide sequence in FIG. 1 (SEQ ID NO:1) or the PLA2G1B polypeptide sequence of FIG. 2 (SEQ ID NO:2). In certain embodiments, a nucleotide sequence substantially identical to the nucleotide sequence of SEQ ID NO:1 is 90% or more identical to the nucleotide sequence of SEQ ID NO:1 or encodes a polypeptide that is 90% or more identical to the polypeptide of SEQ ID NO:2. One test for determining whether two nucleic acids are substantially identical is to determine the percent of identical nucleotide sequences or polypeptide sequences shared between the nucleic acids or polypeptides.

Calculations of sequence identity are often performed as follows. Sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is sometimes 30% or more, 40% or more, 50% or more, often 60% or more, and more often 70%, 80%, 90%, 100% of the length of the reference sequence. The nucleotides or amino acids at corresponding nucleotide or polypeptide positions, respectively, are then compared among the two sequences. When a position in the first sequence is occupied by the same nucleotide or amino acid as the corresponding position in the second sequence, the nucleotides or amino acids are deemed to be identical at that position. The percent identity between the two sequences is a function of the number of identical positions shared by the sequences, taking into account the number of gaps, and the length of each gap, introduced for optimal alignment of the two sequences.

Comparison of sequences and determination of percent identity between two sequences can be accomplished using a mathematical algorithm. Percent identity between two amino acid or nucleotide sequences can be determined using the algorithm of E. Meyers and W. Miller, CABIOS 4: 11-17 (1989), which has been incorporated into the ALIGN program (version 2.0), using a PAM120 weight residue table, a gap length penalty of 12 and a gap penalty of 4. Also, percent identity between two amino acid sequences can be determined using the Needleman and Wunsch, J. Mol. Biol. 48: 444-453 (1970) algorithm which has been incorporated into the GAP program in the GCG software package (available at the http address www.gcg.com), using either a Blossum 62 matrix or a PAM250 matrix, and a gap weight of 16, 14, 12, 10, 8, 6, or 4 and a length weight of 1, 2, 3, 4, 5, or 6. Percent identity between two nucleotide sequences can be determined using the GAP program in the GCG software package (available at http address www.gcg.com), using a NWSgapdna.CMP matrix and a gap weight of 40, 50, 60, 70, or 80 and a length weight of 1, 2, 3, 4, 5, or 6. A set of parameters often used is a Blossum 62 scoring matrix with a gap open penalty of 12, a gap extend penalty of 4, and a frameshift gap penalty of 5.

Another manner for determining if two nucleic acids are substantially identical is to assess whether a polynucleotide homologous to one nucleic acid will hybridize to the other nucleic acid under stringent conditions. As use herein, the term “stringent conditions” refers to conditions for hybridization and washing. Stringent conditions are known to those skilled in the art and can be found in Current Protocols in Molecular Biology, John Wiley & Sons, N.Y. ,6.3.1-6.3.6 (1989). Aqueous and non-aqueous methods are described in that reference and either can be used. An example of stringent hybridization conditions is hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions are 0.5M sodium phosphate, 7% SDS at 65° C., followed by one or more washes at 0.2×SSC, 1% SDS at 65° C.

An example of a substantially identical nucleotide sequence to SEQ ID NO:1 is one that has a different nucleotide sequence and still encodes the polypeptide sequence of SEQ ID NO:2. Another example is a nucleotide sequence that encodes a polypeptide having a polypeptide sequence that is more than 70% identical to, sometimes more than 75%, 80%, or 85% identical to, and often more than 90% and 95% identical to the polypeptide sequence of SEQ ID NO:2.

PLA2G1B nucleotide sequences and polypeptide sequences can be used as “query sequences” to perform a search against public databases to identify other family members or related sequences, for example. Such searches can be performed using the NBLAST and XBLAST programs (version 2.0) of Altschul et al., J. Mol. Biol. 215: 403-10 (1990). BLAST nucleotide searches can be performed with the NBLAST program, score=100, wordlength=12 to obtain nucleotide sequences homologous to PLA2G1B nucleic acid molecules. BLAST polypeptide searches can be performed with the XBLAST program, score=50, wordlength=3 to obtain amino acid sequences homologous to PLA2G1B polypeptides. To obtain gapped alignments for comparison purposes, Gapped BLAST can be utilized as described in Altschul et al., Nucleic Acids Res. 25(17): 3389-3402 (1997). When utilizing BLAST and Gapped BLAST programs, default parameters of the respective programs (e.g., XBLAST and NBLAST) can be used (see the http address www.ncbi.nlm.nih.gov).

A nucleic acid that is substantially identical to the nucleotide sequence of SEQ ID NO:1 may include polymorphic sites at positions equivalent to those described herein (e.g., position 7328 in SEQ ID NO:1) when the sequences are aligned. For example, using the alignment procedures described herein, SNPs in a sequence substantially identical to the sequence of SEQ ID NO:1 can be identified at nucleotide positions that match (i.e., align) with nucleotides at SNP positions in SEQ ID NO:1. Also, where a polymorphic variation is an insertion or deletion, insertion or deletion of a nucleotide sequence from a reference sequence can change the relative positions of other polymorphic sites in the nucleotide sequence.

Substantially identical PLA2G1B nucleotide and polypeptide sequences include those that are naturally occurring, such as allelic variants (same locus), splice variants, homologs (different locus), and orthologs (different organism) or can be non-naturally occurring. Non-naturally occurring variants can be generated by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product). Orthologs, homologs, allelic variants, and splice variants can be identified using methods known in the art. These variants normally comprise a nucleotide sequence encoding a polypeptide that is 50%, about 55% or more, often about 70-75% or more, more often about 80-85% or more, and typically about 90-95% or more identical to the amino acid sequence shown in SEQ ID NO:2 or a fragment thereof. Such nucleic acid molecules can readily be identified as being able to hybridize under stringent conditions to the nucleotide sequence shown in SEQ ID NO:1 or a fragment of this sequence. Nucleic acid molecules corresponding to orthologs, homologs, and allelic variants of the PLA2G1B nucleotide sequence can further be identified by mapping the sequence to the same chromosome or locus as the PLA2G1B nucleotide sequence or variant.

Also, substantially identical PLA2G1B nucleotide sequences may include codons that are altered with respect to the naturally occurring sequence for enhancing expression of a PLA2G1B polypeptide or polypeptide variant in a particular expression system. For example, the nucleic acid can be one in which one or more codons are altered, and often 10% or more or 20% or more of the codons are altered for optimized expression in bacteria (e.g., E. coli.), yeast (e.g., S. cervesiae), human (e.g., 293 cells), insect, or rodent (e.g., hamster) cells.

Fat Deposition Disorder Prognostic and Diagnostic Methods

Methods for prognosing and diagnosing fat deposition, its related disorders (e.g., obesity and NIDDM) and leanness in subjects are provided herein. These methods include detecting the presence or absence of one or more polymorphic variations in a PLA2G1B nucleotide sequence or substantially identical sequence thereof in a sample from a subject, where the presence of a polymorphic variant described herein is indicative of a predisposition to leanness or fat deposition or one or more fat deposition related disorders (e.g., obesity or NIDDM). Determining a predisposition to fat deposition refers to determining whether an individual is at an increased or intermediate risk of fat deposition and determining a predisposition to leanness refers to a decreased risk of fat deposition. Determining a predisposition to NIDDM refers to determining whether an individual is at risk of NIDDM.

Thus, featured herein is a method for detecting a predisposition to fat deposition and a fat deposition disorder, such as obesity and NIDDM, in a subject, which comprises detecting the presence or absence of a polymorphic variation associated with fat deposition at a polymorphic site in a PLA2G1B nucleotide sequence in a nucleic acid sample from a subject, wherein the PLA2G1B nucleotide sequence comprises a polynucleotide sequence selected from the group consisting of: (a) the nucleotide sequence of SEQ ID NO:1; (b) a nucleotide sequence which encodes a polypeptide consisting of the amino acid sequence of SEQ ID NO:2; (c) a nucleotide sequence which encodes a polypeptide that is 90% identical to the amino acid sequence of SEQ ID NO:2 or a nucleotide sequence about 90% or more identical to the nucleotide sequence of SEQ ID NO:1; and (d) a fragment of a nucleotide sequence of (a), (b), or (c) comprising the polymorphic site; whereby the presence of the polymorphic variation is indicative of a predisposition to fat deposition in the subject. In certain embodiments, polymorphic variants at positions 7328 and 9182 are detected for determining a predisposition to fat deposition, a polymorphic variant at position 7256 is detected for determining a predisposition to NIDDM and polymorphic variants at positions in linkage disequilibrium with these positions are detected for determining a predisposition to fat deposition and NIDDM.

Results from prognostic tests may be combined with other test results to diagnose fat deposition related disorders, including NIDDM. For example, prognostic results may be gathered, a patient sample may be ordered based on a determined predisposition to fat deposition or NIDDM, the patient sample is analyzed, and the results of the analysis may be utilized to diagnose the fat deposition related condition (e.g., NIDDM). Also fat deposition diagnostic methods can be developed from studies used to generate prognostic methods in which populations are stratified into subpopulations having different progressions of a fat deposition related disorder or condition.

Predisposition to fat deposition, fat deposition related disorders such as NIDDM and obesity, and leanness sometimes is expressed as a probability, such as an odds ratio, percentage, or risk factor. The predisposition is based upon the presence or absence of one or more polymorphic variants described herein, and also may be based in part upon phenotypic traits of the individual being tested. Methods for calculating predispositions based upon patient data are well known (see, e.g., Agresti, Categorical Data Analysis, 2nd Ed. 2002. Wiley). Allelotyping and genotyping analyses may be carried out in populations other than those exemplified herein to enhance the predictive power of the prognostic method. These further analyses are executed in view of the exemplified procedures described herein, and may be based upon the same polymorphic variations or additional polymorphic variations.

The nucleic acid sample typically is isolated from a biological sample obtained from a subject. For example, nucleic acid can be isolated from blood, saliva, sputum, urine, cell scrapings, and biopsy tissue. The nucleic acid sample can be isolated from a biological sample using standard techniques, such as the technique described in Example 2. As used herein, the term “subject” refers primarily to humans but also refers to other mammals such as dogs, cats, and ungulates (e.g., cattle, sheep, and swine). Subjects also include avians (e.g., chickens and turkeys), reptiles, and fish (e.g., salmon), as embodiments described herein can be adapted to nucleic acid samples isolated from any of these organisms. The nucleic acid sample may be isolated from the subject and then directly utilized in a method for determining the presence of a polymorphic variant, or alternatively, the sample may be isolated and then stored (e.g., frozen) for a period of time before being subjected to analysis.

The presence or absence of a polymorphic variant is determined using one or both chromosomal complements represented in the nucleic acid sample. Determining the presence or absence of a polymorphic variant in both chromosomal complements represented in a nucleic acid sample from a subject having a copy of each chromosome is useful for determining the zygosity of an individual for the polymorphic variant (i.e., whether the individual is homozygous or heterozygous for the polymorphic variant). Any oligonucleotide-based diagnostic may be utilized to determine whether a sample includes the presence or absence of a polymorphic variant in a sample. For example, primer extension methods, ligase sequence determination methods (e.g., U.S. Pat. Nos. 5,679,524 and 5,952,174, and WO 01/27326), mismatch sequence determination methods (e.g., U.S. Pat. Nos. 5,851,770; 5,958,692; 6,110,684; and 6,183,958), microarray sequence determination methods, restriction fragment length polymorphism (RFLP), single strand conformation polymorphism detection (SSCP) (e.g., U.S. Pat. Nos. 5,891,625 and 6,013,499), PCR-based assays (e.g., TAQMAN® PCR System (Applied Biosystems)), and nucleotide sequencing methods may be used.

Oligonucleotide extension methods typically involve providing a pair of oligonucleotide primers in a polymerase chain reaction (PCR) or in other nucleic acid amplification methods for the purpose of amplifying a region from the nucleic acid sample that comprises the polymorphic variation. One oligonucleotide primer is complementary to a region 3′ of the polymorphism and the other is complementary to a region 5′ of the polymorphism. A PCR primer pair may be used in methods disclosed in U.S. Pat. Nos. 4,683,195; 4,683,202, 4,965,188; 5,656,493; 5,998,143; 6,140,054; WO 01/27327; and WO 01/27329 for example. PCR primer pairs may also be used in any commercially available machines that perform PCR, such as any of the GENEAMP® Systems available from Applied Biosystems. Also, those of ordinary skill in the art will be able to design oligonucleotide primers based upon the nucleotide sequence of SEQ ID NO:1 without undue experimentation using knowledge readily available in the art.

Also provided is an extension oligonucleotide that hybridizes to the amplified fragment adjacent to the polymorphic variation. As used herein, the term “adjacent” refers to the 3′ end of the extension oligonucleotide being often 1 nucleotide from the 5′ end of the polymorphic site, and sometimes 2, 3, 4, 5, 6, 7, 8, 9, or 10 nucleotides from the 5′ end of the polymorphic site, in the nucleic acid when the extension oligonucleotide is hybridized to the nucleic acid. The extension oligonucleotide then is extended by one or more nucleotides, and the number and/or type of nucleotides that are added to the extension oligonucleotide determine whether the polymorphic variant is present. Oligonucleotide extension methods are disclosed, for example, in U.S. Pat. Nos. 4,656,127; 4,851,331; 5,679,524; 5,834,189; 5,876,934; 5,908,755; 5,912,118; 5,976,802; 5,981,186; 6,004,744; 6,013,431; 6,017,702; 6,046,005; 6,087,095; 6,210,891; and WO 01/20039. Oligonucleotide extension methods using mass spectrometry are described, for example, in U.S. Pat. Nos. 5,547,835; 5,605,798; 5,691,141; 5,849,542; 5,869,242; 5,928,906; 6,043,031; and 6,194,144, and a method often utilized is described herein in Example 2.

A microarray can be utilized for determining whether a polymorphic variant is present or absent in a nucleic acid sample. A microarray may include any oligonucleotides described herein, and methods for making and using oligonucleotide microarrays suitable for diagnostic use are disclosed in U.S. Pat. Nos. 5,492,806; 5,525,464; 5,589,330; 5,695,940; 5,849,483; 6,018,041; 6,045,996; 6,136,541; 6,142,681; 6,156,501; 6,197,506; 6,223,127; 6,225,625; 6,229,911; 6,239,273; WO 00/52625; WO 01/25485; and WO 01/29259. The microarray typically comprises a solid support and the oligonucleotides may be linked to this solid support by covalent bonds or by non-covalent interactions. The oligonucleotides may also be linked to the solid support directly or by a spacer molecule. A microarray may comprise one or more oligonucleotides complementary to a polymorphic site of SEQ ID NO:1 (e.g., positions 7256, 7328, and/or 9182).

A kit also may be utilized for determining whether a polymorphic variant is present or absent in a nucleic acid sample. A kit often comprises one or more pairs of oligonucleotide primers useful for amplifying a fragment of SEQ ID NO:1 or a substantially identical sequence thereof, where the fragment includes a polymorphic site. The kit sometimes comprises a polymerizing agent, for example, a thermostable nucleic acid polymerase such as one disclosed in U.S. Pat. Nos. 4,889,818 or 6,077,664. Also, the kit often comprises an elongation oligonucleotide that hybridizes to a PLA2G1B nucleic acid in a nucleic acid sample adjacent to the polymorphic site. Where the kit includes an elongation oligonucleotide, it also often comprises chain elongating nucleotides, such as dATP, dTTP, dGTP, dCTP, and dITP, including analogs of dATP, dTTP, dGTP, dCTP and dITP, provided that such analogs are substrates for a thermostable nucleic acid polymerase and can be incorporated into a nucleic acid chain elongated from the extension oligonucleotide. Along with chain elongating nucleotides would be one or more chain terminating nucleotides such as ddATP, ddTTP, ddGTP, ddCTP, and the like. In an embodiment, the kit comprises one or more oligonucleotide primer pairs, a polymerizing agent, chain elongating nucleotides, at least one elongation oligonucleotide, and one or more chain terminating nucleotides. Kits optionally include buffers, vials, microtiter plates, and instructions for use.

Determining the presence of a polymorphic variant, or a combination of two or more polymorphic variants, in a PLA2G1B nucleic acid of the sample is often indicative of a predisposition to fat deposition, leanness, or NIDDM. For example, presence of a guanine at position 7328 of SEQ ID NO:1 in the sense strand of a PLA2G1B nucleotide sequence is associated with an increased risk of fat deposition and presence of an adenine at position 7328 of SEQ ID NO:1 in the sense strand of a PLA2G1B nucleotide sequence is associated with leanness or a decreased risk of fat deposition. Specifically, a subject homozygous for a guanine at position 7328 of SEQ ID NO:1 in the sense strands of the PLA2G1B nucleotide sequence is at a higher risk of fat deposition, a subject heterozygous for a guanine and adenine at position 7328 in the sense strands of the PLA2G1B nucleotide sequence is at an intermediate risk of increased fat deposition, and a subject homozygous for an adenine at position 7328 in the sense strands of the PLA2G1B nucleotide sequence is at a lower risk of fat deposition. Similarly, a subject homozygous for a cytosine at position 7328 in the strands complementary to the sense strands of the PLA2G1B nucleotide sequence is at a higher risk of increased fat deposition, a subject heterozygous for a cytosine and thymine at position 7328 in the strands complementary to the sense strands of the PLA2G1B nucleotide sequence is at an intermediate risk of increased fat deposition, and a subject homozygous for a thymine at position 7328 in the strands complementary to the sense strands of the PLA2G1B nucleotide sequence is at a decreased risk of fat deposition.

Also, presence of a thymine at position 9182 of SEQ ID NO:1 in the sense strand of a PLA2G1B nucleotide sequence is associated with an increased risk of fat deposition and the presence of a guanine at position 9182 in the sense strand of a PLA2G1B nucleotide sequence is associated with leanness or a decreased risk of fat deposition. Specifically, a subject homozygous for a thymine at position 9182 of SEQ ID NO:1 in the sense strands of the PLA2G1B nucleotide sequence is at a higher risk of increased fat deposition, a subject heterozygous for a thymine and guanine at position 9182 in the sense strands of the PLA2G1B nucleotide sequence is at an intermediate risk of increased fat deposition, and a subject homozygous for a guanine at position 9182 in the sense strands of the PLA2G1B nucleotide sequence is at a decreased risk of fat deposition. Similarly, a subject homozygous for an adenine at position 9182 in the strands complementary to the sense strands of the PLA2G1B nucleotide sequence is at a higher risk of increased fat deposition, a subject heterozygous for an adenine and cytosine at position 9182 in the strands complementary to the sense strands of the PLA2G1B nucleotide sequence is at an intermediate risk of increased fat deposition, and a subject homozygous for a guanine at position 9182 in the strands complementary to the sense strands of the PLA2G1B nucleotide sequence is at a lower risk of fat deposition.

Also, the presence of a haplotypes of TTAG and GTAG at positions 4050, 7256, 7328, and 9182, respectively, in the sense strand of a PLA2G1B nucleotide sequence (SEQ ID NO:1) are associated with leanness or a decreased risk of fat deposition. Similarly, the presence of a haplotype of AATC and CATC at positions 4050, 7256, 7328, and 9182, respectively, in the strand complementary to the sense strand of a PLA2G1B nucleotide sequence are associated with leanness.

Presence of a cytosine at position 7256 of SEQ ID NO:1 in the sense strand of a PLA2G1B nucleotide sequence is associated with an increased risk of NIDDM and the presence of a thymine at position 7256 in the sense strand of a PLA2G1B nucleotide sequence is associated with a decreased risk of NIDDM. Specifically, a subject homozygous for a cytosine at position 7256 of SEQ ID NO:1 in the sense strands of the PLA2G1B nucleotide sequence is at a higher risk of NIDDM, a subject heterozygous for a cytosine and thymine at position 7256 in the sense strands of the PLA2G1B nucleotide sequence is at an intermediate risk of NIDDM, and a subject homozygous for a thymine at position 7256 in the sense strands of the PLA2G1B nucleotide sequence is at a decreased risk of NIDDM. Similarly, a subject homozygous for a guanine at position 7256 in the strands complementary to the sense strands of the PLA2G1B nucleotide sequence is at a higher risk of NIDDM, a subject heterozygous for an guanine and adenine at position 7256 in the strands complementary to the sense strands of the PLA2G1B nucleotide sequence is at an intermediate risk of NIDDM, and a subject homozygous for a adenine at position 7256 in the strands complementary to the sense strands of the PLA2G1B nucleotide sequence is at a lower risk of NIDDM.

Applications of Prognostic and Diagnostic Results to Pharmacogenomic Methods

Pharmacogenomics is a discipline that involves tailoring a treatment for a subject according to the subject's genotype as a particular treatment regimen may exert a differential effect depending upon the subject's genotype. Based upon the outcome of a prognostic test described herein, a clinician or physician may target pertinent information and preventative or therapeutic treatments to a subject who would be benefited by the information or treatment and avoid directing such information and treatments to a subject who would not be benefited (e.g., the treatment has no therapeutic effect and/or the subject experiences adverse side effects).

For example, where a candidate therapeutic exhibits a significant interaction with a major allele and a comparatively weak interaction with a minor allele (e.g., an order of magnitude or greater difference in the interaction), such a therapeutic typically would not be administered to a subject genotyped as being homozygous for the minor allele, and sometimes not administered to a subject genotyped as being heterozygous for the minor allele. In another example, where a candidate therapeutic is not significantly toxic when administered to subjects who are homozygous for a major allele but is comparatively toxic when administered to subjects heterozygous or homozygous for a minor allele, the candidate therapeutic is not typically administered to subjects who are genotyped as being heterozygous or homozygous with respect to the minor allele.

The prognostic methods described herein are applicable to pharmacogenomic methods for preventing, alleviating or treating fat deposition conditions such as obesity and NIDDM. For example, a nucleic acid sample from an individual may be subjected to a prognostic test described herein. Where one or more polymorphic variations associated with increased risk of obesity or NIDDM are identified in a subject, information for preventing or treating obesity or NIDDM and/or one or more obesity or NIDDM treatment regimens then may be prescribed to that subject. For example, a patient having a cytosine at position 7256 in SEQ ID NO: 1 often is prescribed a preventative regimen designed to minimize the occurrence of NIDDM.

In certain embodiments, a treatment regimen is specifically prescribed and/or administered to individuals who will most benefit from it based upon their risk of developing obesity or NIDDM assessed by the prognostic methods described herein. Thus, provided are methods for identifying a subject predisposed to obesity or NIDDM and then prescribing a therapeutic or preventative regimen to individuals identified as having a predisposition. Thus, certain embodiments are directed to a method for reducing fat deposition, obesity or NIDDM in a subject, which comprises: detecting the presence or absence of a polymorphic variant associated with fat deposition, obesity or NIDDM in a PLA2G1B nucleotide sequence in a nucleic acid sample from a subject, where the PLA2G1B nucleotide sequence comprises a polynucleotide sequence selected from the group consisting of: (a) the polynucleotide sequence of SEQ ID NO:1; (b) a polynucleotide sequence which encodes a polypeptide consisting of the amino acid sequence of SEQ ID NO:2; (c) a polynucleotide sequence which encodes a polypeptide that is 90% identical to the amino acid sequence of SEQ ID NO:2; and (d) a fragment of a polynucleotide sequence of (a), (b), or (c); and prescribing or administering a treatment regimen to a subject from whom the sample originated where the presence of a polymorphic variation associated with fat reduction is detected in the PLA2G1B nucleotide sequence. In these methods, predisposition results may be utilized in combination with other test results to diagnose fat deposition associated conditions, such as obesity, metabolic conditions (e.g., NIDDM) and cardiovascular conditions (e.g., myocardial infarction).

The treatment sometimes is preventative (e.g., is prescribed or administered to reduce the probability that a fat deposition associated condition arises or progresses), sometimes is therapeutic, and sometimes delays, alleviates or halts the progression of a fat deposition associated condition. Any known preventative or therapeutic treatment for alleviating or preventing the occurrence of a fat deposition associated disorder is prescribed and/or administered. For example, the treatment sometimes is or includes a drug that reduces fat deposition, including, for example, an appetite suppressant (e.g., Phentermine, Adipex, Bontril, Didrex, Ionamin, Meridia, Phendimetrazine, Tenuate, Sibutramine), a lipase inhibitor (e.g., Olistat), a phospholipase inhibitor, a PLA2G1B nucleic acid, a PLA2G1B polypeptide, and/or a molecule that interacts with a PLA2G1B nucleic acid or PLA2G1B polypeptide described hereafter. In another example, the treatment is or includes a physical exercise regimen, dietary counseling and/or a dietary regimen (e.g., a low fat diet and/or a diet where the subject eats during pre-scheduled intervals) optionally coupled with dietary counseling, psychological counseling and/or psychotherapy, and sometimes optionally coupled with prescription of a psychotherapeutic or psychoprophylactic (e.g., an antidepressant or anti-anxiety therapeutic). In other embodiments directed to diabetes management, a subject sometimes is prescribed a regimen for regularly monitoring blood glucose levels, dietary counseling, a dietary regimen for managing blood glucose levels, and/or a blood glucose altering drug regimen. Examples of blood glucose altering drug regimens are regular administration of insulin (e.g., injection, pump, inhaler spray, nasal spray, insulin patch, and insulin tablet), and administration of hypoglycemics (e.g., glyburide or repaglinide), starch blockers (e.g., acarbose), liver glucose regulating agents (e.g., metformin), and/or insulin sensitizers (e.g., rosiglitzaone or pioglitazone). Prescription and/or administration of each treatment or combinations of treatments often is dependent upon the age of the subject as well as the subject's physiological, medical, and/or psychological condition.

In an embodiment, the pharmacogenomic methods described herein are applicable to subjects who are women about forty or more years of age and have not yet entered menopause, undergoing menopause, or post-menopausal. Those subjects identified as having an increased risk for fat deposition sometimes are prescribed a hormone replacement treatment (HRT) regimen. There are many HRT regimens known in the art, which include regular administration of estrogen (e.g., Prumarin®), progesterone (e.g., Provera®)), androgen (e.g., testosterone), a combination of estrogen and progesterone, a combination of estrogen and androgen (e.g., Estratest®), growth hormone, dehydroepiandrosterone (DHEA), a sulfate ester of DHEA, or a combination of DHEA and a DHEA sulfate ester. Also, selective estrogen receptor modulators (SERMS) such as raloxifene and tamoxifen, for example, can be prescribed. Those women diagnosed as having an increased risk of fat deposition sometimes are prescribed an estrogen replacement therapy (ERT) regimen or SERMs regimen as an alternative to a combination of estrogen and progesterone, due to an association between ERT and lower fat deposition and an association between increased fat deposition and progesterone replacement therapy.

In another embodiment, pharmacogenomic methods are applicable to subjects who are women using a contraceptive or are contemplating use of a contraceptive, where the contraceptive has been shown to increase fat deposition in subjects. This embodiment often applies to women who are pre-pubescent, who are in puberty, or who are post-pubescent and pre-menopausal. Many oral contraceptives, especially those that include higher contents of estrogen compared to other oral contraceptives, have been shown to increase fat deposition in subjects. Those subjects identified as having an increased risk for fat deposition by the methods described herein often are advised not to begin an oral contraceptive regimen or to discontinue an oral contraceptive regimen. Alternatively, subjects identified as having an increased risk for fat deposition sometimes are advised to begin an oral contraceptive regimen using a contraceptive having lower estrogen content as compared to other available oral contraceptives (e.g., Allesse®, Levlite®, Loestrin-Fe®, and Mircette® are examples of contraceptives having lower estrogen content).

As therapeutic approaches for obesity or NIDDM continue to evolve and improve, the goal of treatments for fat deposition related disorders is to intervene even before clinical signs (e.g., impaired glucose tolerance) first manifest. Thus, genetic markers associated with susceptibility to obesity or NIDDM prove useful for early diagnosis, prevention and treatment of obesity or NIDDM.

As obesity or NIDDM preventative and treatment information can be specifically targeted to subjects in need thereof (e.g. those at risk of developing obesity or NIDDM or those that have early stages of obesity or NIDDM), provided herein is a method for preventing or reducing the risk of developing obesity or NIDDM in a subject, which comprises: (a) detecting the presence or absence of a polymorphic variation associated with obesity or NIDDM at a polymorphic site in a nucleotide sequence in a nucleic acid sample from a subject; (b) identifying a subject with a predisposition to obesity or NIDDM, whereby the presence of the polymorphic variation is indicative of a predisposition to obesity or NIDDM in the subject; and (c) if such a predisposition is identified, providing the subject with information about methods or products to prevent or reduce obesity or NIDDM or to delay the onset of obesity or NIDDM. Also provided is a method of targeting information or advertising to a subpopulation of a human population based on the subpopulation being genetically predisposed to a disease or condition, which comprises: (a) detecting the presence or absence of a polymorphic variation associated with obesity or NIDDM at a polymorphic site in a nucleotide sequence in a nucleic acid sample from a subject; (b) identifying the subpopulation of subjects in which the polymorphic variation is associated with obesity or NIDDM; and (c) providing information only to the subpopulation of subjects about a particular product which may be obtained and consumed or applied by the subject to help prevent or delay onset of the disease or condition.

Pharmacogenomics methods also may be used to analyze and predict a response to an obesity or NIDDM treatment or a drug. For example, if pharmacogenomics analysis indicates a likelihood that an individual will respond positively to a obesity or NIDDM treatment with a particular drug, the drug may be administered to the individual. Conversely, if the analysis indicates that an individual is likely to respond negatively to treatment with a particular drug, an alternative course of treatment may be prescribed. A negative response may be defined as either the absence of an efficacious response or the presence of toxic side effects. The response to a therapeutic treatment can be predicted in a background study in which subjects in any of the following populations are genotyped: a population that responds favorably to a treatment regimen, a population that does not respond significantly to a treatment regimen, and a population that responds adversely to a treatment regiment (e.g. exhibits one or more side effects). These populations are provided as examples and other populations and subpopulations may be analyzed. Based upon the results of these analyses, a subject is genotyped to predict whether he or she will respond favorably to a treatment regimen, not respond significantly to a treatment regimen, or respond adversely to a treatment regimen.

The prognostic tests described herein also are applicable to clinical drug trials. One or more polymorphic variants indicative of response to an agent for treating obesity or NIDDM or to side effects to an agent for treating obesity or NIDDM may be identified using the methods described herein. Thereafter, potential participants in clinical trials of such an agent may be screened to identify those individuals most likely to respond favorably to the drug and exclude those likely to experience side effects. In that way, the effectiveness of drug treatment may be measured in individuals who respond positively to the drug, without lowering the measurement as a result of the inclusion of individuals who are unlikely to respond positively in the study and without risking undesirable safety problems.

Thus, another embodiment is a method of selecting an individual for inclusion in a clinical trial of a treatment or drug comprising the steps of: (a) obtaining a nucleic acid sample from an individual; (b) determining the identity of a polymorphic variation which is associated with a positive response to the treatment or the drug, or at least one polymorphic variation which is associated with a negative response to the treatment or the drug in the nucleic acid sample, and (c) including the individual in the clinical trial if the nucleic acid sample contains said polymorphic variation associated with a positive response to the treatment or the drug or if the nucleic acid sample lacks said polymorphic variation associated with a negative response to the treatment or the drug. In addition, the methods of the present invention for selecting an individual for inclusion in a clinical trial of a treatment or drug encompass methods with any further limitation described in this disclosure, or those following, specified alone or in any combination. The polymorphic variation may be in a sequence selected individually or in any combination from the group consisting of (i) a polynucleotide sequence set forth in SEQ ID NO: 1; (ii) a polynucleotide sequence that is 90% identical to a nucleotide sequence set forth in SEQ ID NO: 1; (iii) a polynucleotide sequence that encodes a polypeptide having an amino acid sequence identical to or 90% identical to an amino acid sequence encoded by a nucleotide sequence set forth in SEQ ID NO: 1; and (iv) a fragment of a polynucleotide sequence of (i), (ii), or (iii) comprising the polymorphic site. The including step (c) optionally comprises administering the drug or the treatment to the individual if the nucleic acid sample contains the polymorphic variation associated with a positive response to the treatment or the drug and the nucleic acid sample lacks said biallelic marker associated with a negative response to the treatment or the drug.

Also provided herein is a method of partnering between a diagnostic/prognostic testing provider and a provider of a consumable product, which comprises: (a) the diagnostic/prognostic testing provider detects the presence or absence of a polymorphic variation associated with obesity or NIDDM at a polymorphic site in a nucleotide sequence in a nucleic acid sample from a subject; (b) the diagnostic/prognostic testing provider identifies the subpopulation of subjects in which the polymorphic variation is associated with obesity or NIDDM; (c) the diagnostic/prognostic testing provider forwards information to the subpopulation of subjects about a particular product which may be obtained and consumed or applied by the subject to help prevent or delay onset of the disease or condition; and (d) the provider of a consumable product forwards to the diagnostic test provider a fee every time the diagnostic/prognostic test provider forwards information to the subject as set forth in step (c) above.

Methods for Identifying Candidate Therapeutics for Reducing Fat Deposition and Treating Related Disorders

Current therapies for the treatment of NIDDM have limited efficacy, limited tolerability and significant mechanisms-based side effects, including weight gain and hypoglycaemia. Few of the available therapies adequately address underlying defects such as obesity and insulin resistance. Thus, newer approaches are desperately needed (Moller D. Nature. 414:821-827 (2001)). Current therapeutic approaches were largely developed in the absence of defined molecular targets or even a solid understanding of disease pathogenesis. The same holds true for the treatment of obesity, where treatments have limited lasting effects and many side effects. Therefore, there is a need for methods of identifying candidate therapeutics that target the biochemical pathways related to the development of obesity and/or diabetes.

Featured herein is a method for identifying candidate therapeutics for reducing fat deposition and/or the development of NIDDM. The method comprises contacting a test molecule with a PLA2G1B nucleic acid, nucleic acid variant, polypeptide, or polypeptide variant in a system. The nucleic acid is often the PLA2G1B nucleotide sequence represented by SEQ ID NO:1, sometimes a nucleotide sequence that is substantially identical to the nucleotide sequence of SEQ ID NO:1, or sometimes a fragment thereof, and the PLA2G1B polypeptide is a polypeptide encoded by any of these nucleic acids. The method also comprises determining the presence or absence of an interaction between the test molecule and the PLA2G1B nucleic acid or polypeptide, where the presence of an interaction between the test molecule and the PLA2G1B nucleic acid or polypeptide identifies the test molecule as a candidate therapeutic for fat reduction or NIDDM.

As used herein, the term “test molecule” and “candidate therapeutic” refers to modulators of regulation of transcription and translation of PLA2G1B nucleic acids and modulations of expression and activity of PLA2G1B polypeptides. The term “modulator” as used herein refers to a molecule which agonizes or antagonizes PLA2G1B DNA replication and/or DNA processing (e.g., methylation), PLA2G1B RNA transcription and/or RNA processing (e.g., removal of intronic sequences and/or translocation from the nucleus), PLA2G1B polypeptide production (e.g., translation of the polypeptide from mRNA, and/or post-translational modification such as glycosylation, phosphorylation, and proteolysis of pro-polypeptides), and/or PLA2G1B function (e.g., conformational changes, binding of nucleotides or nucleotide analogs, binding and/or translocation of ions, interaction with binding partners, effect on membrane potential, effect on fat deposition, effect on metabolic condition, and/or effect on cardiovascular condition). Test molecules and candidate therapeutics include, but are not limited to, compounds, antisense nucleic acids, ribozymes, PLA2G1B polypeptide or fragments thereof, immunotherapeutics (e.g., antibodies).

Compounds

Compounds may be utilized as test molecules for identifying candidate therapeutics for reducing fat deposition or treating NIDDM. Compounds can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive (see, e.g., Zuckermann, R. N. et al., J. Med. Chem. 37: 2678-85 (1994)); spatially addressable parallel solid phase or solution phase libraries; synthetic library methods requiring deconvolution; “one-bead one-compound” library methods; and synthetic library methods using affinity chromatography selection. Biological library and peptoid library approaches are typically limited to peptide libraries, while the other approaches are applicable to peptide, non-peptide oligomer or small molecule libraries of compounds (Lam, Anticancer Drug Des. 12: 145, (1997)). Examples of methods for synthesizing molecular libraries are described, for example, in DeWitt et al., Proc. Natl. Acad. Sci. USA. 90: 6909 (1993); Erb et al., Proc. Natl. Acad. Sci. USA 91: 11422 (1994); Zuckermann et al., J. Med. Chem. 37: 2678 (1994); Cho et al., Science 261: 1303 (1993); Carrell et al., Angew. Chem. Int. Ed. Engl. 33: 2059 (1994); Carell et al., Angew. Chem. Int. Ed. Engl. 33: 2061 (1994); and in Gallop et al., J. Med. Chem. 37: 1233 (1994).

Libraries of compounds may be presented in solution (e.g., Houghten, Biotechniques 13: 412-421 (1992)), or on beads (Lam, Nature 354: 82-84 (1991)), chips (Fodor, Nature 364: 555-556 (1993)), bacteria or spores (Ladner, U.S. Pat. No.5,223,409), plasmids (Cull et al., Proc. Natl. Acad. Sci. USA 89: 1865-1869 (1992)) or on phage (Scott and Smith, Science 249: 386-390 (1990); Devlin, Science 249: 404-406 (1990); Cwirla et al., Proc. Natl. Acad. Sci. 87: 6378-6382 (1990); Felici, J. Mol. Biol. 222: 301-310 (1991); Ladner supra.).

Compounds may alter expression or activity of PLA2G1B polypeptides and may be a small molecule. Small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Compounds that modulate PLA2 functions are known. For example, U.S. Pat. Nos. 5,504,073; 5,578,639; and 5,968,963 are directed to intestinal PLA2 inhibitors; U.S. Pat. No. 5,622,828 is directed to secretory PLA2 polypeptide inhibitors; U.S. Pat. No.4,978,609 is directed to pancreatic PLA2 inhibitors; and U.S. Pat. Nos. 5,567,597; 5,308,766; 5,352,673; and 5,427,919 are directed to general PLA2 inhibitors. Other PLA2 inhibitors are described in: U.S. Publication No. 20020065246A1; U.S. Pat. Nos. 6,350,892; 6,310,217; 6,180,596; 6,177,257; 6,147,100; 6,110,933; 5,994,398; 5,972,972; 5,968,818; 5,866,318; 5,817,826; 5,688,821; 5,679,801; 5,656,602; 5,597,943; 5,563,164; 5,523,297; 5,508,302; 5,453,443; 5,451,600; 5,446,189; 5,427,919; 5,420,289; 5,391,817; 5,350,579; 5,290,817; 5,281,626; 5,229,403; 5,208,244; 5,208,223; 5,202,350; 5,145,844; 5,141,959; 5,124,334; 5,120,647; 5,112,864; 5,075,339; 5,070,207; 5,066,671; 4,959,357; 4,845,292; 4,239,780; and WO 02/08189; WO 00/71118; WO 00/27824; WO 99/44604; WO 99/41278; WO 99/29726; WO 98/33797; WO 98/25893; WO 98/24437; WO 98/08818; and WO 97/17448. Compounds known or tested as not significantly bioavailable in the serum are often tested in screening assays.

Antisense Nucleic Acid Molecules, Ribozymes, and Modified PLA2G1B Nucleic Acid Molecules

Also featured herein are antisense, ribozyme, and modified PLA2G1B nucleic acids for use as test molecules in methods for identifying candidate therapeutics for reducing fat deposition and treating related disorders, e.g., diabetes. An “antisense” nucleic acid refers to a nucleotide sequence which is complementary to a “sense” nucleic acid encoding a polypeptide, e.g., complementary to the coding strand of a double-stranded cDNA molecule or complementary to an mRNA sequence. The antisense nucleic acid can be complementary to an entire PLA2G1B coding strand, or to only a portion thereof (e.g., the coding region of human PLA2G1B corresponding to SEQ ID NO:1). In another embodiment, the antisense nucleic acid molecule is antisense to a “noncoding region” of the coding strand of a nucleotide sequence encoding PLA2G1B (e.g., 5′ and 3′ untranslated regions).

An antisense nucleic acid can be designed such that it is complementary to the entire coding region of PLA2G1B mRNA, and often the antisense nucleic acid is an oligonucleotide that is antisense to only a portion of a coding or noncoding region of PLA2G1B mRNA. For example, the antisense oligonucleotide can be complementary to the region surrounding the translation start site of PLA2G1B mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length. The antisense nucleic acids, which include the ribozymes described hereafter, can be designed to target PLA2G1B nucleic acid or PLA2G1B nucleic acid variants. Among the variants, minor alleles and major alleles can be targeted, and those associated with a higher risk to fat deposition, such as alleles having a guanine at position 7328 and/or a thymine at position 9182, are often designed, tested, and administered to subjects.

An antisense nucleic acid can be constructed using chemical synthesis and enzymatic ligation reactions using standard procedures. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

Antisense nucleic acids are typically administered to a subject (e.g., by direct injection at a tissue site) or generated in situ such that they hybridize with or bind to cellular mRNA and/or genomic DNA encoding a PLA2G1B polypeptide and thereby inhibit expression of the polypeptide, for example, by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, for example, by linking antisense nucleic acid molecules to peptides or antibodies which bind to cell surface receptors or antigens. Antisense nucleic acid molecules can also be delivered to cells using the vectors described herein. Sufficient intracellular concentrations of antisense molecules are achieved by incorporating a strong promoter, such as a pol II or pol III promoter, in the vector construct.

Antisense nucleic acid molecules are sometimes α-anomeric nucleic acid molecules. An α-anomeric nucleic acid molecule forms specific double-stranded hybrids with complementary RNA in which, contrary to the usual β-units, the strands run parallel to each other (Gaultier et al., Nucleic Acids. Res. 15: 6625-6641 (1987)). Antisense nucleic acid molecules can also comprise a 2′-o-methylribonucleotide (Inoue et al., Nucleic Acids Res. 15: 6131-6148 (1987)) or achimeric RNA-DNA analogue (Inoue et al., FEBS Lett. 215: 327-330 (1987)).

In another embodiment, an antisense nucleic acid is a ribozyme. A ribozyme having specificity for a PLA2G1B-encoding nucleic acid can include one or more sequences complementary to the nucleotide sequence of a PLA2G1B DNA sequence disclosed herein (e.g., SEQ ID NO:1), and a sequence having a known catalytic sequence responsible for mRNA cleavage (see U.S. Pat. No. 5,093,246 or Haselhoff and Gerlach, Nature 334: 585-591 (1988)). For example, a derivative of a Tetrahymena L-19 IVS RNA is sometimes utilized in which the nucleotide sequence of the active site is complementary to the nucleotide sequence to be cleaved in a PLA2G1B-encoding mRNA. See, e.g., Cech et al U.S. Pat. No. 4,987,071; and Cech et al U.S. Pat. No.5,116,742. Also, PLA2G1B mRNA can be used to select a catalytic RNA having a specific ribonuclease activity from a pool of RNA molecules. See, e.g., Bartel & Szostak, Science 261: 1411-1418 (1993).

PLA2G1B gene expression can be inhibited by targeting nucleotide sequences complementary to the regulatory region of the PLA2G1B (e.g., PLA2G1B promoter and/or enhancers) to form triple helical structures that prevent transcription of the PLA2G1B gene in target cells. See, Helene, Anticancer Drug Des. 6(6): 569-84 (1991); Helene et al., Ann. N.Y Acad. Sci. 660: 27-36 (1992); and Maher, Bioassays 14(12): 807-15 (1992). Potential sequences that can be targeted for triple helix formation can be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

Antisense, ribozyme, and modified PLA2G1B nucleic acid molecules can be altered at base moieties, sugar moieties or phosphate backbone moieties to improve stability, hybridization, or solubility of the molecule. For example, the deoxyribose phosphate backbone of nucleic acid molecules can be modified to generate peptide nucleic acids (see Hyrup et al., Bioorganic & Medicinal Chemistry 4 (1): 5-23 (1996)). As used herein, the terms “peptide nucleic acid” or “PNA” refers to a nucleic acid mimic such as a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. Synthesis of PNA oligomers can be performed using standard solid phase peptide synthesis protocols as described, for example, in Hyrup et al., (1996) supra and Perry-O'Keefe et al., Proc. Natl. Acad. Sci. 93: 14670-675 (1996).

PNAs of PLA2G1B nucleic acids can be used in therapeutic and diagnostic applications. For example, PNAs can be used as antisense or antigene agents for sequence-specific modulation of gene expression by, for example, inducing transcription or translation arrest or inhibiting replication. PNAs of PLA2G1B nucleic acid molecules can also be used in the analysis of single base pair mutations in a gene, (e.g., by PNA-directed PCR clamping); as “artificial restriction enzymes” when used in combination with other enzymes, (e.g., S1 nucleases (Hyrup (1996) supra)); or as probes or primers for DNA sequencing or hybridization (Hyrup et al., (1996) supra; Perry-O'Keefe supra).

In other embodiments, oligonucleotides may include other appended groups such as peptides (e.g., for targeting host cell receptors in vivo), or agents facilitating transport across cell membranes (see, e.g., Letsinger et al., Proc. Natl. Acad. Sci. USA 86: 6553-6556 (1989); Lemaitre et al., Proc. Natl. Acad. Sci. USA 84: 648-652 (1987); PCT Publication No. W088/09810) or the blood-brain barrier (see, e.g., PCT Publication No. W089/10134). In addition, oligonucleotides can be modified with hybridization-triggered cleavage agents (See, e.g., Krol et al., Bio-Techniques 6: 958-976 (1988)) or intercalating agents. (See, e.g., Zon, Pharm. Res. 5: 539-549 (1988) ). To this end, the oligonucleotide may be conjugated to another molecule, (e.g., a peptide, hybridization triggered cross-linking agent, transport agent, or hybridization-triggered cleavage agent).

Also included herein are molecular beacon oligonucleotide primer and probe molecules having one or more regions which are complementary to a PLA2G1B nucleic acid of the invention, two complementary regions one having a fluorophore and one a quencher such that the molecular beacon is useful for quantifying the presence of the PLA2G1B nucleic acid of the invention in a sample. Molecular beacon nucleic acids are described, for example, in Lizardi et al., U.S. Pat. No. 5,854,033; Nazarenko et al., U.S. Pat. No. 5,866,336, and Livak et al., U.S. Pat. 5,876,930.

Anti-PLA2G1B Antibodies

In an embodiment, antibodies are screened as test molecules and used as therapeutics for reducing fat deposition or treating NIDDM in a subject. The term “antibody” as used herein refers to an immunoglobulin molecule or immunologically active portion thereof, i.e., an antigen-binding portion. Examples of immunologically active portions of immunoglobulin molecules include F(ab) and F(ab′)₂ fragments which can be generated by treating the antibody with an enzyme such as pepsin. An antibody can be a polyclonal, monoclonal, recombinant, e.g., a chimeric or humanized, fully human, non-human, e.g., murine, or single chain antibody. An antibody may have effector function and can fix complement, and is sometimes coupled to a toxin or imaging agent.

A full-length PLA2G1B polypeptide or, antigenic peptide fragment of PLA2G1B can be used as an immunogen or can be used to identify anti-PLA2G1B antibodies made with other immunogens, e.g., cells, membrane preparations, and the like. The antigenic peptide of PLA2G1B should include at least 8 amino acid residues of the amino acid sequence shown in SEQ ID NO:2 and encompasses an epitope of PLA2G1B. Antigenic peptides include 10 or more amino acids, 15 or more amino acids, often 20 or more amino acids, and typically 30 or more amino acids. Hydrophilic and hydrophobic fragments of PLA2G1B polypeptides can be used as immunogens.

Epitopes encompassed by the antigenic peptide are regions of PLA2G1B located on the surface of the polypeptide (e.g., hydrophilic regions) as well as regions with high antigenicity. For example, an Emini surface probability analysis of the human PLA2G1B polypeptide sequence can be used to indicate the regions that have a particularly high probability of being localized to the surface of the PLA2G1B polypeptide and are thus likely to constitute surface residues useful for targeting antibody production. The antibody may bind an epitope on any domain or region on PLA2G1B polypeptides described herein.

Also, chimeric, humanized, and completely human antibodies are useful for applications which include repeated administration to subjects. Chimeric and humanized monoclonal antibodies, comprising both human and non-human portions, can be made using standard recombinant DNA techniques. Such chimeric and humanized monoclonal antibodies can be produced by recombinant DNA techniques known in the art, for example using methods described in Robinson et al International Application No. PCT/US86/02269; Akira, et al European Patent Application 184,187; Taniguchi, M., European Patent Application 171,496; Morrison et al European Patent Application 173,494; Neuberger et al PCT International Publication No. WO 86/01533; Cabilly et al U.S. Pat. No. 4,816,567; Cabilly et al European Patent Application 125,023; Better et al., Science 240: 1041-1043 (1988); Liu et al., Proc. Natl. Acad. Sci. USA 84: 3439-3443 (1987); Liu et al., J. Immunol. 139: 3521-3526 (1987); Sun et al., Proc. Natl. Acad. Sci. USA 84: 214-218 (1987); Nishimura et al., Canc. Res. 47: 999-1005 (1987); Wood et al., Nature 314: 446449 (1985); and Shaw et al., J. Natl. Cancer Inst. 80: 1553-1559 (1988); Morrison, S. L., Science 229: 1202-1207 (1985); Oi et al., BioTechniques 4: 214 (1986); Winter U.S. Pat. No. 5,225,539; Jones et al., Nature 321: 552-525 (1986); Verhoeyan et al., Science 239: 1534; and Beidler et al., J. Immunol. 141: 4053-4060 (1988).

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes. See, for example, Lonberg and Huszar, Int. Rev. Immunol. 13: 65-93 (1995); and U.S. Pat. Nos. 5,625,126; 5,633,425; 5,569,825; 5,661,016; and 5,545,806. In addition, companies such as Abgenix, Inc. (Fremont, Calif.) and Medarex, Inc. (Princeton, N.J.), can be engaged to provide human antibodies directed against a selected antigen using technology similar to that described above. Completely human antibodies that recognize a selected epitope also can be generated using a technique referred to as “guided selection.” In this approach a selected non-human monoclonal antibody (e.g., a murine antibody) is used to guide the selection of a completely human antibody recognizing the same epitope. This technology is described for example by Jespers et al., Bio/Technology 12: 899-903 (1994).

An anti-PLA2G1B antibody can be a single chain antibody. A single chain antibody (scFV) can be engineered (see, e.g., Colcher, D. et al., Ann. NY Acad. Sci. 880: 263-80 (1999); and Reiter, Y., Clin. Cancer Res. 2: 245-52 (1996)). Single chain antibodies can be dimerized or multimerized to generate multivalent antibodies having specificities for different epitopes of the same target PLA2G1B polypeptide.

Antibodies also may be selected or modified so that they exhibit reduced or no ability to bind an Fc receptor. For example, an antibody may be an isotype or subtype, fragment or other mutant, which does not support binding to an Fc receptor (e.g., it has a mutagenized or deleted Fc receptor binding region).

Also, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Antibody conjugates can be used for modifying a given biological response. For example, the drug moiety may be a protein or polypeptide possessing a desired biological activity. Such proteins may include, for example, a toxin such as abrin, ricin A, pseudomonas exotoxin, or diphtheria toxin; a polypeptide such as tumor necrosis factor, α-interferon, β-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors. Also, an antibody can be conjugated to a second antibody to form an antibody heteroconjugate as described by Segal in U.S. Pat. No. 4,676,980, for example.

An anti-PLA2G1B antibody (e.g. monoclonal antibody) can be used to isolate PLA2G1B polypeptides by standard techniques, such as affinity chromatography or immunoprecipitation. Moreover, an anti-PLA2G1B antibody can be used to detect a PLA2G1B polypeptide (e.g., in a cellular lysate or cell supernatant) in order to evaluate the abundance and pattern of expression of the polypeptide. Anti-PLA2G1B antibodies can be used diagnostically to monitor polypeptide levels in tissue as part of a clinical testing procedure, e.g., to determine the efficacy of a given treatment regimen. Detection can be facilitated by coupling (i.e., physically linking) the antibody to a detectable substance (i.e., antibody labeling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H. Also, an anti-PLA2G1B antibody can be utilized as a test molecule for determining whether it can reduce fat deposition or treat a related disorder, e.g., diabetes, and as a therapeutic for administration to a subject for reducing fat deposition or for treating a related metabolic disorder such as diabetes. Monoclonal antibodies against type I PLA2 molecules have been reported (U.S. Pat. No. 5,767,249).

An antibody can be made by immunizing with a purified PLA2G1B antigen, or a fragment thereof, e.g., a fragment described herein, a membrane associated antigen, tissues, e.g., crude tissue preparations, whole cells, preferably living cells, lysed cells, or cell fractions.

Included herein are antibodies which bind only a native PLA2G1B polypeptide, only denatured or otherwise non-native PLA2G1B polypeptide, or which bind both, as well as those having linear or conformational epitopes. Conformational epitopes sometimes can be identified by selecting antibodies that bind to native but not denatured PLA2G1B polypeptide.

Screening Assays

Featured herein is a method for identifying a candidate therapeutic for fat reduction and/or treating NIDDM, which comprises (a) introducing a test molecule to a system which comprises a nucleic acid comprising a PLA2G1B nucleotide sequence selected from the group consisting of: (i) the nucleotide sequence of SEQ ID NO:1; (ii) a nucleotide sequence which encodes a polypeptide consisting of the amino acid sequence of SEQ ID NO:2; (iii) a nucleotide sequence which encodes a polypeptide that is 90% identical to the amino acid sequence of SEQ ID NO:2; and (iv) a fragment of a nucleotide sequence of (i), (ii), or (iii); or introducing a test molecule to a system which comprises a protein encoded by a nucleotide sequence of (i), (ii), (iii), or (iv); and (b) determining the presence or absence of an interaction between the test molecule and the nucleic acid or protein, where the presence of an interaction between the test molecule and the nucleic acid or protein identifies the test molecule as a candidate therapeutic for fat reduction.

As used herein, the term “system” refers to a cell free in vitro environment and a cell-based environment such as a collection of cells, a tissue, an organ, or an organism. A system is “contacted” with a test molecule in a variety of manners, including adding molecules in solution and allowing them to interact with one another by diffusion, cell injection, and any administration routes in an animal. As used herein, the term “interaction” refers to an effect of a test molecule on a PLA2G1B nucleic acid, polypeptide, or variant thereof (collectively referred to as a “PLA2G1B molecule”), where the effect is sometimes binding between the test molecule and the nucleic acid or polypeptide, and is often an observable change in cells, tissue, or organism.

There are many standard methods for detecting the presence or absence of interaction between a test molecule and a PLA2G1B nucleic acid or polypeptide. For example, titrametric, acidimetric, radiometric, NMR, monolayer, polarographic, spectrophotometric, fluorescent, and ESR assays probative of PLA2 function are described in Reynolds et al., Methods in Enzymology 197: 3-23 (1991); Yu et al., Methods in Enzymology 197: 65-75 (1991); Reynolds et al., Analytical Biochemistry 204:190-197 (1992); Reynolds et al., Anal. Biochem. 217:25-32 (1994); Yang et al., Anal. Biochem. 269:278-288 (1999); U.S. Pat. No.5,464,754; and WO 00/34791.

An interaction can be determined by labeling the test molecule and/or the PLA2G1B molecule, where the label is covalently or non-covalently attached to the test molecule or PLA2G1B molecule. The label is sometimes a radioactive molecule such as ¹²⁵I, ¹³¹I, ³⁵S or ³H, which can be detected by direct counting of radioemission or by scintillation counting. Also, enzymatic labels such as horseradish peroxidase, alkaline phosphatase, or luciferase may be utilized where the enzymatic label can be detected by determining conversion of an appropriate substrate to product. Also, presence or absence of an interaction can be determined without labeling. For example, a microphysiometer (e.g., Cytosensor) is an analytical instrument that measures the rate at which a cell acidifies its environment using a light-addressable potentiometric sensor (LAPS). Changes in this acidification rate can be used as an indication of an interaction between a test molecule and PLA2G1B (McConnell, H. M. et al., Science 257: 1906-1912 (1992)).

In cell-based systems, cells typically include a PLA2G1B nucleic acid or polypeptide or variants thereof and are often of mammalian origin, although the cell can be of any origin. Whole cells, cell homogenates, and cell fractions (e.g., cell membrane fractions) can be subjected to analysis. Where interactions between a test molecule with a PLA2G1B polypeptide or variant thereof are monitored, soluble and/or membrane bound forms of the polypeptide or variant may be utilized. Where membrane-bound forms of the polypeptide are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl-N,N-dimethyl-3-ammonio-1-propane sulfonate.

An interaction between two molecules can also be detected by monitoring fluorescence energy transfer (FET) (see, for example, Lakowicz et al., U.S. Pat. No. 5,631,169; Stavrianopoulos, et al. U.S. Pat. No.4,868,103). A fluorophore label on a first, “donor” molecule is selected such that its emitted fluorescent energy will be absorbed by a fluorescent label on a second, “acceptor” molecule, which in turn is able to fluoresce due to the absorbed energy. Alternately, the “donor” polypeptide molecule may simply utilize the natural fluorescent energy of tryptophan residues. Labels are chosen that emit different wavelengths of light, such that the “acceptor” molecule label may be differentiated from that of the “donor”. Since the efficiency of energy transfer between the labels is related to the distance separating the molecules, the spatial relationship between the molecules can be assessed. In a situation in which binding occurs between the molecules, the fluorescent emission of the “acceptor” molecule label in the assay should be maximal. An FET binding event can be conveniently measured through standard fluorometric detection means well known in the art (e.g., using a fluorimeter).

In another embodiment, determining the presence or absence of an interaction between a test molecule and a PLA2G1B molecule can be effected by using real-time Biomolecular Interaction Analysis (BIA) (see, e.g., Sjolander & Urbaniczk, Anal. Chem. 63: 2338-2345 (1991) and Szabo et al., Curr. Opin. Struct. Biol. 5: 699-705 (1995)). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In another embodiment, the PLA2G1B molecule or test molecules are anchored to a solid phase. The PLA2G1B molecule/test molecule complexes anchored to the solid phase can be detected at the end of the reaction. The target PLA2G1B molecule is often anchored to a solid surface, and the test molecule, which is not anchored, can be labeled, either directly or indirectly, with detectable labels discussed herein.

It may be desirable to immobilize a PLA2G1B molecule, an anti-PLA2G1B antibody, or test molecules to facilitate separation of complexed from uncomplexed forms of PLA2G1B molecules and test molecules, as well as to accommodate automation of the assay. Binding of a test molecule to a PLA2G1B molecule can be accomplished in any vessel suitable for containing the reactants. Examples of such vessels include microtitre plates, test tubes, and micro-centrifuge tubes. In one embodiment, a fusion polypeptide can be provided which adds a domain that allows a PLA2G1B molecule to be bound to a matrix. For example, glutathione-S-transferase/PLA2G1B fusion polypeptides or glutathione-S-transferase/target fusion polypeptides can be adsorbed onto glutathione sepharose beads (Sigma Chemical, St. Louis, Mo.) or glutathione derivatized microtitre plates, which are then combined with the test compound or the test compound and either the non-adsorbed target polypeptide or PLA2G1B polypeptide, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of PLA2G1B binding or activity determined using standard techniques.

Other techniques for immobilizing a PLA2G1B molecule on matrices include using biotin and streptavidin. For example, biotinylated PLA2G1B polypeptide or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with PLA2G1B polypeptide or test molecules but which do not interfere with binding of the PLA2G1B polypeptide to its test molecule. Such antibodies can be derivatized to the wells of the plate, and unbound target or PLA2G1B polypeptide trapped in the wells by antibody conjugation. Methods for detecting such complexes, in addition to those described above for the GST-immobilized complexes, include immunodetection of complexes using antibodies reactive with the PLA2G1B polypeptide or target molecule, as well as enzyme-linked assays which rely on detecting an enzymatic activity associated with the PLA2G1B polypeptide or test molecule.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., Trends Biochem Sci August;18(8): 284-7 (1993)); chromatography (gel filtration chromatography, ion-exchange chromatography); electrophoresis (see, e.g., Ausubel, F. et al., eds. Current Protocols in Molecular Biology, J. Wiley: New York (1999)); and immunoprecipitation (see, for example, Ausubel, F. et al., eds. Current Protocols in Molecular Biology, J. Wiley: New York (1999)). Such resins and chromatographic techniques are known to one skilled in the art (see, e.g., Heegaard, N. H., J Mol. Recognit. Winter; 11(1-6): 141-8 (1998); Hage, D. S., and Tweed, S. A., J. Chromatogr. B Biomed. Sci. Appl. October 10; 699 (1-2): 499-525 (1997)). Further, fluorescence energy transfer may also be conveniently utilized, as described herein, to detect binding without further purification of the complex from solution.

In another embodiment, modulators of PLA2G1B expression are identified. For example, a cell or cell free mixture is contacted with a candidate compound and the expression of PLA2G1B mRNA or polypeptide evaluated relative to the level of expression of PLA2G1B mRNA or polypeptide in the absence of the candidate compound. When expression of PLA2G1B mRNA or polypeptide is greater in the presence of the candidate compound than in its absence, the candidate compound is identified as a stimulator of PLA2G1B mRNA or polypeptide expression. Alternatively, when expression of PLA2G1B mRNA or polypeptide is less (statistically significantly less) in the presence of the candidate compound than in its absence, the candidate compound is identified as an inhibitor of PLA2G1B mRNA or polypeptide expression. The level of PLA2G1B mRNA or polypeptide expression can be determined by methods described herein for detecting PLA2G1B mRNA or polypeptide.

PLA2G1B Binding Partners

In another embodiment, binding partners that interact with a PLA2G1B molecule are detected. The PLA2G1B molecules can interact with one or more cellular or extracellular macromolecules, such as polypeptides, in vivo, and these molecules that interact with PLA2G1B molecules are referred to herein as “binding partners.” Molecules that disrupt such interactions can be useful in regulating the activity of the target gene product. Such molecules can include, but are not limited to molecules such as antibodies, peptides, and small molecules. The preferred target genes/products for use in this embodiment are the PLA2G1B genes herein identified. In an alternative embodiment, the invention provides methods for determining the ability of the test compound to modulate the activity of a PLA2G1B polypeptide through modulation of the activity of a downstream effector of a PLA2G1B target molecule. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), e.g., a substrate, a reaction mixture containing the target gene product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface (e.g., a microtitre plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. For example, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

Also, binding partners of PLA2G1B molecules can be identified in a two-hybrid assay or three-hybrid assay (see, e.g., U.S. Pat. No. 5,283,317; Zervos et al., Cell 72:223-232 (1993); Madura et al., J. Biol. Chem. 268: 12046-12054 (1993); Bartel et al., Biotechniques 14: 920-924 (1993); Iwabuchi et al., Oncogene 8: 1693-1696 (1993); and Brent WO94/10300), to identify other polypeptides, which bind to or interact with PLA2G1B (“PLA2G1B-binding polypeptides” or “PLA2G1B-bp”) and are involved in PLA2G1B activity. Such PLA2G1B-bps can be activators or inhibitors of signals by the PLA2G1B polypeptides or PLA2G1B targets as, for example, downstream elements of a PLA2G1B-mediated signaling pathway.

A two-hybrid system is based on the modular nature of most transcription factors, which consist of separable DNA-binding and activation domains. Briefly, the assay utilizes two different DNA constructs. In one construct, the gene that codes for a PLA2G1B polypeptide is fused to a gene encoding the DNA binding domain of a known transcription factor (e.g., GAL-4). In the other construct, a DNA sequence, from a library of DNA sequences, that encodes an unidentified polypeptide (“prey” or “sample”) is fused to a gene that codes for the activation domain of the known transcription factor. (Alternatively the: PLA2G1B polypeptide can be the fused to the activator domain.) If the “bait” and the “prey” polypeptides are able to interact, in vivo, forming a PLA2G1B-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the polypeptide which interacts with the PLA2G1B polypeptide.

Identification of Candidate Therapeutics

Candidate therapeutics for reducing fat deposition or treating a related disorder (e.g., diabetes) are identified from a group of test molecules that interact with a PLA2G1B nucleic acid or polypeptide. Test molecules are normally ranked according to the degree with which they interact or modulate (e.g., agonize or antagonize) DNA replication and/or processing, RNA transcription and/or processing, polypeptide production and/or processing, and/or function of PLA2G1B molecules, for example, and then top ranking modulators are selected. Also, pharmacogenomic information described herein can determine the rank of a modulator. Candidate therapeutics typically are formulated for administration to a subject.

Therapeutic Treatments

Formulations or pharmaceutical compositions typically include in combination with a pharmaceutically acceptable carrier a compound, an antisense nucleic acid, an siRNA molecule capable of inhibiting the expression of PLA2G1B or, optionally, any of its transcripts, a ribozyme, an antibody, a binding partner that interacts with a PLA2G1B polypeptide, a PLA2G1B nucleic acid, or a fragment thereof. The formulated molecule may be one that is identified by a screening method described above. Also, formulations may comprise a PLA2G1B polypeptide or fragment thereof and a pharmaceutically acceptable carrier. As used herein, the term “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

As explained above, secreted PLA2 polypeptides can exert an effect on digestion. Triglycerides are the main source for fat deposition in animals and enter the small intestine from the stomach typically as an emulsion. When bile acids from the gall bladder are mixed with such an emulsion, micelles are formed, where triglycerides are encapsulated in the center of the micelles and the outer surface of the micelles is composed of polar moieties such as phospholipid, cholesterol, and bile salts. Bile acids can also form similar structures, such as emulsified lipid droplets, multi- and uni-lamellar vesicles, and mixed micelles. Triglycerides situated in the center of these structures are protected from the hydrolytic action of pancreatic lipase and colipase by the bipolar outer layer. When PLA2 polypeptides are secreted and enter the digestive system (e.g., the small intestine, lower intestine, and the stomach), they hydrolyze phospholipids into free fatty acids and lysophospholipids. Hydrolysis of the phospholipid can disrupt the micelles and similar structures, thereby releasing triglycerides into the digestive system and rendering them subject to lipase-mediated hydrolysis into fat-forming free fatty acids. Also, the lysophospholipids released by the hydrolyzed phospholipids have mild detergent properties and result in smaller micelles than formed by the phospholipids that encapsulate triglycerides. These smaller micelles are more susceptible to lipase degradation, which hydrolyzes the encapsulated triglycerides into fat-forming fatty acids.

Thus, inhibiting secreted PLA2 molecules such as PLA2G1B can reduce fat deposition in a direct and indirect manner. Specifically, an inhibitor of a secreted PLA2 molecule (1) can directly reduce phospholipid hydrolysis, which decreases the concentration of free triglyceride available for lipase-mediated hydrolysis into free fatty acids due to reduced micelle disruption, and (2) can indirectly reduce the amount of smaller micelles formed by lysophospholipid, thereby reducing the concentration of lipase-mediated release of free fatty acids from triglycerides.

An inhibitor of a secreted PLA2 molecule (e.g., PLA2G1B) often interacts with its target in the digestive tract, especially in the small intestine, the large intestine, and in the stomach. Bioavailability in the serum therefore is not required for inhibition of a secreted PLA2 molecule in the digestive tract. “Bioavailability” often refers to a serum concentration of a compound over a period of time following a certain route of administration in comparison to intravenous administration, the latter of which is characterized by 100% bioavailability. There are several known analytical methods for determining serum bioavailability of a substance (e.g., HPLC, LC/MS, and radioimmunoassay), and any of these methods may be utilized to determine serum bioavailability. Modulators having an undetectable serum bioavailability are often utilized when targeting a secreted phospholipase such as PLA2G1B, as serum availability can lead to undesirable side effects, and modulators having a serum bioavailability of 2% or less, 5% or less, 10% or less, 15% or less, 20% or less, or 25% or less (compared to the total amount of modulator administered) are sometimes utilized. It is also possible that modulators having a serum availability of 30% or more, 40% or more, 50% or more, 60% or more, 70% or more, 80% or more, or 90% or more are utilized as phospholipase may be targeted in regions outside the digestive tract. For example, as fatty acids are known to stimulate PPAR-gamma expression and thereby cause adipocyte differentiation, a phospholipase such as PLA2G1B can be inhibited outside the digestive tract to reduce adipocyte differentiation.

Lipase inhibitors such as orlistat reduce free fatty acid release from triglycerides, and can thereby reduce fat deposition in subjects. Subjects who have experienced decreased fat deposition in response to administration of a lipase inhibitor are desirable candidates for determining whether a specific PLA2 inhibitor reduces fat deposition. Thus, determining lipase inhibitor response can be utilized as a parameter for screening subjects in studies that evaluate the effect of specific PLA2 inhibitors on fat deposition.

One side effect of orlistat is steatorrhea, presumably due to an increased triglyceride content in the stool. Large amounts of lipophilic substance such as triglyceride in the bowel can disrupt the fecal matrix and prevent the formation of firm, formed stools as evidenced by the stool-softening effects of liquid petrolactum and oils such as olive oil (Curry, Laxative products; In: Handbook of Nonprescription Drugs, pp. 75-97; American Pharmaceutical Association, Washington, D.C. (1986)). This disruption of the stool matrix produces stools that are looser than normal and that, in some cases, may be perceived as diarrhea. As compared to administering a lipase inhibitor alone, administering a phospholipase inhibitor to a subject, or a phospholipase inhibitor in conjunction with a lipase inhibitor, can modify stool composition (e.g., decreases triglyceride content) and thereby solidify the stool. Thus, an unfortunate side effect of a lipase inhibitor may be overcome by administering a phospholipase inhibitor in conjunction with a lipase inhibitor, or by administering a phospholipase inhibitor without a lipase inhibitor. Other side effects that may be also overcome by such a therapeutic strategy include oily spotting, flatus with discharge, increased defecation, fecal incontinence, and vitamin A and vitamin D deficiencies. Stool samples from a subject administered a phospholipase inhibitor may be characterized any time after the phospholipase inhibitor is administered, for example, 1, 2, 3, 4, 5, 10, 15, 20, 24, or 48 or more hours after administration.

Thus, featured herein is a method for reducing fat deposition in a subject, which comprises administering to a subject a molecule that inhibits the function of a PLA2G1B polypeptide in the digestive tract of the subject. Also featured herein is a method for reducing fat deposition in a subject, which comprises administering to a subject a molecule that inhibits a PLA2G1B polypeptide, where the subject does not experience significant steatorrhea after the molecule is administered or where the molecule induces less steatorrhea in subjects as compared to steatorrhea caused in subjects by a lipase inhibitor, whereby inhibition of the PLA2G1B polypeptide reduces fat deposition in the subject. The digestive tract of the subject includes, for example, the small intestine, large intestine, stomach, pancreas, and gall bladder. The molecule is often a compound, and the compound is often not significantly biovailable in the serum of the subject. The term “function of a PLA2G1B polypeptide” or “activity of a PLA2G1B polypeptide” as used herein refers to catalytic hydrolysis of phospholipid and/or binding of a PLA2G1B polypeptide to a binding partner, for example. A compound may inhibit PLA2G1B function, for example, by competing with phospholipid at the active site of the phospholipase, by reducing trypsin-catalyzed cleavage of pro-PLA2G1B into the active form of PLA2G1B, and/or by reducing the probability that a PLA2G1B polypeptide interacts with a binding partner. The inhibitory molecule may be administered by any of the methods described hereafter and it is often orally administered to the subject. The molecule can be administered before, during, or after a meal, and may be formulated in liquid or solid dosage form. Also, the subject may be administered or self-administer an inhibitor of PLA2G1B function, or an inhibitor of PLA2G1B function in conjunction with a lipase inhibitor (e.g., orlistat), a colipase inhibitor, or a combination thereof.

The effect of a molecule on stool consistency may be assessed in a subject to determine whether the subject experiences significant steatorrhea after administering the molecule, and may be compared to stool consistency for subjects administered a lipase inhibitor such as orlistat. Molecules leading to a firmer stool than stool from subjects administered a lipase inhibitor are sometimes subjected to further testing in subjects. A subset of subjects administered the molecule may not experience significant steatorrhea, and sometimes 60% or fewer, 50% or fewer, 40% or fewer, 30% of fewer, 20% or fewer, 10% or fewer, or 5% or fewer subjects will experience significant steatorrhea after the molecule is administered. To determine whether a subject experiences significant steatorrhea, stool samples may be characterized in terms of viscosity (e.g., peak force units, McRorie et al., Regul. Toxicol. Pharmacol. 31: 59-67 (2000)); jejeunal villous height, fecal mass, fecal fat content, and bile acid content (Vuoristo & Miettinen, Scand. J. Gastroenterol. 22: 289-294 (1987)); and triglyceride, fatty acid, and phospholipid content, for example. In terms of viscosity, firm stool samples are sometimes 1300 or more peak force units (PF); loose stool samples are sometimes 600 PF or less, 500 PF or less, or 400 PF or less, and at times 300 PF or less or 200 PF or less; and stool samples are sometimes 600 PF or more, 700 PF or more, 800 PF or more; 900 PF or more, 1000 PF or more, 1100 PF or more, or 1200 PF or more after a PLA2G1B inhibitor is administered to a subject. Also, fecal fat may be characterized as described in Van de Kamer et al., J. Biol. Chem. 177:347-355 (1949) and can range from 0.5 to 25 g/100 g stool (e.g., 0.5 or more, 2 or more 5 or more, 10 or more, 15 or more or 20 or more), and fecal bile acid concentration may be characterized as described in Vuoristo et al., Gastroenterology 78:1518-1525 (1980) and can range from 0.01 to 20 mM (e.g., 0.1 or more, 1 or more, 5 or more, 10 or more, or 15 or more) after a PLA2G1B Vuoristo & Mittinen, supra, and can range from 50 to 200 grams per day (e.g., 50 or more, 100 or more, or 150 or more). In addition, fecal fat can be quantified in the range of 2 to 7 grams per day or zero to 19% by weight using known methods, and can be qualitatively assessed from a Sudan staining test, where a normal range for neutral fats is less than 60 droplets/HPF and where a normal range of total fats (i.e., neutral, soaps, and fatty acids) is less than 100 droplets/HPF.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. Molecules can also be prepared in the form of suppositories (e.g., with conventional suppository bases such as cocoa butter and other glycerides) or retention enemas for rectal delivery.

In one embodiment, active molecules are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Molecules which exhibit high therapeutic indices are preferred. While molecules that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such molecules lies preferably within a range of circulating concentrations that include the ED₅₀ with little or no toxicity. The dosage may vary within this range depending upon the dosage form employed and the route of administration utilized. For any molecules used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, sometimes about 0.01 to 25 mg/kg body weight, often about 0.1 to 20 mg/kg body weight, and more often about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, sometimes between 2 to 8 weeks, often between about 3 to 7 weeks, and more often for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

With regard to polypeptide formulations, featured herein is a method for reducing fat deposition or treating NIDDM in a subject, which comprises contacting a PLA2G1B protein with one or more cells of a subject in need thereof, wherein the PLA2G1B protein is encoded by a PLA2G1B nucleotide sequence which comprises a polynucleotide sequence selected from the group consisting of: (a) the polynucleotide sequence of SEQ ID NO:1; (b) a polynucleotide sequence which encodes a polypeptide consisting of the amino acid sequence of SEQ ID NO:2; (c) a polynucleotide sequence which is 90% or more identical to the nucleotide sequence of SEQ ID NO:1 or which encodes a polypeptide that is 90% or more identical to the amino acid sequence of SEQ ID NO:2; and (d) a fragment of one of the foregoing polynucleotide sequences, where contacting the one or more cells of the subject with the PLA2G1B protein reduces fat deposition, alleviates obesity and/or alleviates NIDDM. The PLA2G1B protein often is administered to a subject prognosed as being at risk of fat deposition, obesity and/or NIDDM or is diagnosed as having obesity or NIDDM before the protein is administered in vivo (e.g., injected into the subject), ex vivo (e.g., cells from the subject are contacted with the protein in a petri dish and the contacted cells then are returned to the subject), or in vitro (e.g., cells from the subject are contacted with the protein in a petri dish to observe the effect of the protein on the cells). The subject often is a human.

For antibodies, a dosage of 0.1 mg/kg of body weight (generally 10 mg/kg to 20 mg/kg) is often utilized. If the antibody is to act in the brain, a dosage of 50 mg/kg to 100 mg/kg is often appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al., J. Acquired Immune Deficiency Syndromes and Human Retrovirology 14:193 (1997).

Antibody conjugates can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a polypeptide such as tumor necrosis factor, .alpha.-interferon, .beta.-interferon, nerve growth factor, platelet derived growth factor, tissue plasminogen activator; or, biological response modifiers such as, for example, lymphokines, interleukin-1 (“IL-1″), interleukin-2 (“IL-2″), interleukin-6 (“IL-6″), granulocyte macrophage colony stimulating factor (“GM-CSF”), granulocyte colony stimulating factor (“G-CSF”), or other growth factors. Alternatively, an antibody can be conjugated to a second antibody to form an antibody heteroconjugate as described by Segal in U.S. Pat. No. 4,676,980.

For compounds, exemplary doses include milligram or microgram amounts of the compound per kilogram of subject or sample weight, for example, about 1 microgram per kilogram to about 500 milligrams per kilogram, about 100 micrograms per kilogram to about 5 milligrams per kilogram, or about 1 microgram per kilogram to about 50 micrograms per kilogram. It is understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

PLA2G1B nucleic acid molecules can be inserted into vectors and used in gene therapy methods for reducing fat deposition or treating NIDDM. Featured herein is a method for reducing fat deposition, alleviating obesity and/or alleviating NIDDM in a subject, which comprises contacting a PLA2G1B nucleic acid with one or more cells of a subject in need thereof, wherein the PLA2G1B nucleic acid comprises a polynucleotide sequence selected from the group consisting of (a) the polynucleotide sequence of SEQ ID NO:1; (b) a polynucleotide sequence which encodes a polypeptide consisting of the amino acid sequence of SEQ ID NO:2; (c) a polynucleotide sequence which encodes a polypeptide that is 90% identical to the amino acid sequence of SEQ ID NO:2 or a polynucleotide sequence 90% identical to the nucleotide sequence of SEQ ID NO:1; and (d) a fragment of one of the foregoing polynucleotide sequences, where contacting the one or more cells of the subject with the PLA2G1B protein reduces fat deposition, alleviates obesity and/or alleviates NIDDM. The PLA2G1B nucleic acid often is administered to a subject prognosed as being at risk of fat deposition, obesity and/or NIDDM or is diagnosed as having obesity or NIDDM before the nucleic acid is administered in vivo, ex vivo, or in vitro. The subject often is a human.

Gene therapy vectors can be delivered to a subject by, for example, intravenous injection, local administration (see U.S. Pat. No. 5,328,470) or by stereotactic injection (see e.g., Chen et al., (1994) Proc. Natl. Acad. Sci. USA 91:3054-3057). Pharmaceutical preparations of gene therapy vectors can include a gene therapy vector in an acceptable diluent, or can comprise a slow release matrix in which the gene delivery vehicle is imbedded. Alternatively, where the complete gene delivery vector can be produced intact from recombinant cells (e.g., retroviral vectors) the pharmaceutical preparation can include one or more cells which produce the gene delivery system. Examples of gene delivery vectors are described herein.

Pharmaceutical compositions can be included in a container, pack, or dispenser together with instructions for administration.

Pharmaceutical compositions of active ingredients can be administered by any of the paths described herein for therapeutic and prophylactic methods for reducing fat deposition or treating NIDDM. With regard to both prophylactic and therapeutic methods of treatment, such treatments may be specifically tailored or modified, based on knowledge obtained from pharmacogenomic analyses described herein. As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, small molecules, peptides, antibodies, ribozymes and antisense oligonucleotides.

Administration of a prophylactic agent can occur prior to the manifestation of symptoms characteristic of the PLA2G1B aberrance, such that a disease or disorder is prevented or, alternatively, delayed in its progression. Depending on the type of PLA2G1B aberrance, for example, a PLA2G1B molecule, PLA2G1B agonist, or PLA2G1B antagonist agent can be used for treating the subject. The appropriate agent can be determined based on screening assays described herein.

As discussed, successful treatment of PLA2G1B disorders can be brought about by techniques that serve to inhibit the expression or activity of target gene products. For example, compounds (e.g., an agent identified using an assays described above or an siRNA molecule) that exhibit negative modulatory activity can be used in accordance with the invention to prevent and/or ameliorate fat deposition or diabetes. Such molecules can include, but are not limited to peptides, phosphopeptides, small organic or inorganic molecules, or antibodies (including, for example, polyclonal, monoclonal, humanized, anti-idiotypic, chimeric or single chain antibodies, and FAb, F(ab′)₂ and FAb expression library fragments, scFV molecules, and epitope-binding fragments thereof).

Further, antisense and ribozyme molecules that inhibit expression of the target gene can also be used in accordance with the invention to reduce the level of target gene expression, thus effectively reducing the level of target gene activity. Still further, triple helix molecules can be utilized in reducing the level of target gene activity. Antisense, ribozyme and triple helix molecules are discussed above.

It is possible that the use of antisense, ribozyme, and/or triple helix molecules to reduce or inhibit mutant gene expression can also reduce or inhibit the transcription (triple helix) and/or translation (antisense, ribozyme) of mRNA produced by normal target gene alleles, such that the concentration of normal target gene product present can be lower than is necessary for a normal phenotype. In such cases, nucleic acid molecules that encode and express target gene polypeptides exhibiting normal target gene activity can be introduced into cells via gene therapy method. Alternatively, in instances in that the target gene encodes an extracellular polypeptide, it can be preferable to co-administer normal target gene polypeptide into the cell or tissue in order to maintain the requisite level of cellular or tissue target gene activity.

PLA2G1B gene expression sometimes can be inhibited by the introduction of double-stranded RNA (dsRNA), which induces potent and specific gene silencing, a phenomenon called RNA interference or RNAi. See, e.g., Fire et al., U.S. Pat. No. 6,506,559; Tuschl et al. PCT International Publication No. WO 01/75164; Kay et al. PCT International Publication No. WO 03/010180A1; or Bosher J M, Labouesse, Nat Cell Biol February 2000;2(2):E31-6. This process has been improved by decreasing the size of the double-stranded RNA to 20-24 base pairs (to create small-interfering RNAs or siRNAs) that “switched off” genes in mammalian cells without initiating an acute phase response, i.e., a host defense mechanism that often results in cell death. See, e.g., Caplen et al. Proc Natl Acad Sci USA. Aug. 14, 2001;98(17):9742-7 and Elbashir S M et al. Methods 2002February; 26(2):199-213.

There is increasing evidence that post-transcriptional gene silencing by RNA interference (RNAI) for inhibiting targeted expression in mammalian cells at the mRNA level is effective in human cells. There is additional evidence of effective methods for inhibiting the proliferation and migration of tumor cells in human patients, and for inhibiting metastatic cancer development. See, e.g., U.S. patent application No. US2001000993183; Caplen N J et al. Proc Natl Acad Sci USA; and Abderrahmani A. et al. Mol Cell Biol Nov. 21, 2001(21):7256-67.

An “siRNA” or “RNAi” refers to a nucleic acid that forms a double stranded RNA and has the ability to reduce or inhibit expression of a gene or target gene when the siRNA is delivered to or expressed in the same cell as the gene or target gene. “siRNA” thus refers to short double stranded RNA formed by the complementary strands. Complementary portions of the siRNA that hybridize to form the double stranded molecule often have substantial or complete identity to the target molecule sequence. In one embodiment, an siRNA refers to a nucleic acid that has substantial or complete identity to a target gene and forms a double stranded siRNA, such as a nucleotide sequence in SEQ ID NO: 1, for example.

When designing the siRNA molecules, the targeted region often is selected from a given DNA sequence beginning 50 to 100 nt downstream of the start codon. See, e.g., Elbashir et al,. Methods 26:199-213 (2002). Initially, 5′ or 3′ UTRs and regions nearby the start codon were avoided assuming that UTR-binding proteins and/or translation initiation complexes may interfere with binding of the siRNP or RISC endonuclease complex. Sometimes regions of the target 23 nucleotides in length conforming to the sequence motif AA(N19)TT (N, an nucleotide), and regions with approximately 30% to 70% G/C-content (often about 50% G/C-content) often are selected. If no suitable sequences are found, the search often is extended using the motif NA(N21). The sequence of the sense siRNA sometimes corresponds to (N19) TT or N21 (position 3 to 23 of the 23-nt motif), respectively. In the latter case, the 3′ end of the sense siRNA often is converted to TT. The rationale for this sequence conversion is to generate a symmetric duplex with respect to the sequence composition of the sense and antisense 3′ overhangs. The antisense siRNA is synthesized as the complement to position 1 to 21 of the 23-nt motif. Because position 1 of the 23-nt motif is not recognized sequence-specifically by the antisense siRNA, the 3′-most nucleotide residue of the antisense siRNA can be chosen deliberately. However, the penultimate nucleotide of the antisense siRNA (complementary to position 2 of the 23-nt motif) often is complementary to the targeted sequence. For simplifying chemical synthesis, TT often is utilized. siRNAs corresponding to the target motif NAR(N17)YNN, where R is purine (A,G) and Y is pyrimidine (C,U), often are selected. Respective 21 nucleotide sense and antisense siRNAs often begin with a purine nucleotide and can also be expressed from pol III expression vectors without a change in targeting site. Expression of RNAs from pol III promoters often is efficient when the first transcribed nucleotide is a purine.

The sequence of the siRNA can correspond to the full length target gene, or a subsequence thereof. Often, the siRNA is about 15 to about 50 nucleotides in length (e.g., each complementary sequence of the double stranded siRNA is 15-50 nucleotides in length, and the double stranded siRNA is about 15-50 base pairs in length, somtimes about 20-30 nucleotides in length or about 20-25 nucleotides in length, e.g., 20, 21, 22, 23, 24, 25, 26,27,28, 29, or 30 nucleotides in length. The siRNA often is about 21 nucleotides in length. Methods of using siRNA are well known in the art, and specific siRNA molecules may be purchased from a number of companies including Dharmacon Research, Inc.

Another method by which nucleic acid molecules may be utilized in treating or preventing a disease characterized by PLA2G1B expression is through the use of aptamer molecules specific for PLA2G1B polypeptide. Aptamers are nucleic acid molecules having a tertiary structure which permits them to specifically bind to polypeptide ligands (see, e.g., Osborne, et al., Curr. Opin. Chem. Biol. 1(1): 5-9 (1997); and Patel, D. J., Curr. Opin. Chem. Biol. June;1(1): 32-46 (1997)). Since nucleic acid molecules may in many cases be more conveniently introduced into target cells than therapeutic polypeptide molecules may be, aptamers offer a method by which PLA2G1B polypeptide activity may be specifically decreased without the introduction of drugs or other molecules which may have pluripotent effects.

Antibodies can be generated that are both specific for target gene product and that reduce target gene product activity. Such antibodies may, therefore, by administered in instances whereby negative modulatory techniques are appropriate for the treatment of PLA2G1B disorders. For a description of antibodies, see the Antibody section above.

In circumstances where injection of an animal or a human subject with a PLA2G1B polypeptide or epitope for stimulating antibody production is harmful to the subject, it is possible to generate an immune response against PLA2G1B through the use of anti-idiotypic antibodies (see, for example, Herlyn, D., Ann. Med.; 31(1): 66-78 (1999); and Bhattacharya-Chatterjee, M., and Foon, K. A., Cancer Treat. Res.; 94: 51-68 (1998)). If an anti-idiotypic antibody is introduced into a mammal or human subject, it should stimulate the production of anti-anti-idiotypic antibodies, which should be specific to the PLA2G1B polypeptide. Vaccines directed to a disease characterized by PLA2G1B expression may also be generated in this fashion.

In instances where the target antigen is intracellular and whole antibodies are used, internalizing antibodies may be preferred. Lipofectin or liposomes can be used to deliver the antibody or a fragment of the Fab region that binds to the target antigen into cells. Where fragments of the antibody are used, the smallest inhibitory fragment that binds to the target antigen is preferred. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see e.g., Marasco et al., Proc. Natl. Acad. Sci. USA 90: 7889-7893 (1993)).

PLA2G1B molecules and compounds that inhibit target gene expression, synthesis and/or activity can be administered to a patient at therapeutically effective doses to prevent, treat or ameliorate PLA2G1B disorders. A therapeutically effective dose refers to that amount of the compound sufficient to result in amelioration of symptoms of the disorders.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD_{50/}ED₅₀. Compounds that exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

Data obtained from cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of effective dose determination for an individual is the ability to directly assay levels of “free” and “bound” compound in the serum of the test subject. Such assays may utilize antibody mimics and/or “biosensors” that have been created through molecular imprinting techniques. The compound which is able to modulate PLA2G1B activity is used as a template, or “imprinting molecule”, to spatially organize polymerizable monomers prior to their polymerization with catalytic reagents. The subsequent removal of the imprinted molecule leaves a polymer matrix which contains a repeated “negative image” of the compound and is able to selectively rebind the molecule under biological assay conditions. A detailed review of this technique can be seen in Ansell, R. J. et al., Current Opinion in Biotechnology 7: 89-94 (1996) and in Shea, K. J., Trends in Polymer Science 2: 166-173 (1994). Such “imprinted” affinity matrixes are amenable to ligand-binding assays, whereby the immobilized monoclonal antibody component is replaced by an appropriately imprinted matrix. An example of the use of such matrixes in this way can be seen in Vlatakis, G. et al., Nature 361: 645-647 (1993). Through the use of isotope-labeling, the “free” concentration of compound which modulates the expression or activity of PLA2G1B can be readily monitored and used in calculations of IC₅₀. Such “imprinted” affinity matrixes can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized based on its individual IC₅₀. A rudimentary example of such a “biosensor” is discussed in Kriz, D. et al., Analytical Chemistry 67: 2142-2144 (1995).

Provided herein are methods of modulating PLA2G1B expression or activity for therapeutic purposes. Accordingly, in an exemplary embodiment, the modulatory method of the invention involves contacting a cell with a PLA2G1B or agent that modulates one or more of the activities of PLA2G1B polypeptide activity associated with the cell. An agent that modulates PLA2G1B polypeptide activity can be an agent as described herein, such as a nucleic acid or a polypeptide, a naturally-occurring target molecule of a PLA2G1B polypeptide (e.g., a PLA2G1B substrate or receptor), a PLA2G1B antibody, a PLA2G1B agonist or antagonist, a peptidomimetic of a PLA2G1B agonist or antagonist, or other small molecule.

In one embodiment, the agent stimulates one or more PLA2G1B activities. Examples of such stimulatory agents include active PLA2G1B polypeptide and a nucleic acid molecule encoding PLA2G1B. In another embodiment, the agent inhibits one or more PLA2G1B activities. Examples of such inhibitory agents include antisense PLA2G1B nucleic acid molecules, anti-PLA2G1B antibodies, and PLA2G1B inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a PLA2G1B polypeptide or nucleic acid molecule. In one embodiment, the method involves administering an agent (e.g., an agent identified by a screening assay described herein), or combination of agents that modulates (e.g., upregulates or downregulates) PLA2G1B expression or activity. In another embodiment, the method involves administering a PLA2G1B polypeptide or nucleic acid molecule as therapy to compensate for reduced, aberrant, or unwanted PLA2G1B expression or activity.

Stimulation of PLA2G1B activity is desirable in situations in which PLA2G1B is abnormally downregulated and/or in which increased PLA2G1B activity is likely to have a beneficial effect. For example, stimulation of PLA2G1B activity is desirable in situations in which a PLA2G1B is downregulated and/or in which increased PLA2G1B activity is likely to have a beneficial effect. Likewise, inhibition of PLA2G1B activity is desirable in situations in which PLA2G1B is abnormally upregulated and/or in which decreased PLA2G1B activity is likely to have a beneficial effect.

Featured herein are methods of causing or inducing a desired biological response in an individual comprising the steps of: providing or administering to an individual a composition comprising a polypeptide, of the invention, or a fragment thereof, or a therapeutic formulation described herein, wherein said biological response is selected from the group consisting of:
(a) modulating circulating (either blood, serum or plasma) levels (concentration) of glucose, wherein said modulating is preferably lowering;
(b) increasing cell or tissue sensitivity to insulin, particularly muscle, adipose, liver or brain;
(c) inhibiting the progression from impaired glucose tolerance to insulin resistance;
(d) increasing glucose uptake in skeletal muscle cells;
(e) increasing glucose uptake in adipose cells;
(f) increasing glucose uptake in neuronal cells;
(g) increasing glucose uptake in red blood cells;
(h) increasing glucose uptake in the brain; and
(i) significantly reducing the postprandial increase in plasma glucose following a meal, particularly a high carbohydrate meal. In further preferred embodiments, the pharmaceutical or physiologically acceptable composition may be used as an insulin sensitiser.

In further preferred embodiments, the pharmaceutical or physiologically acceptable composition can be used in a method to improve insulin sensitivity in some persons with Non-Insulin Dependent Diabetes Mellitus (NIDDM) in combination with insulin therapy.

In further preferred embodiments, the pharmaceutical or physiologically acceptable composition can be used in a method to improve insulin sensitivity in some persons with Non-Insulin Dependent Diabetes Mellitus (NIDDM) without insulin therapy.

In further preferred embodiments, the pharmaceutical or physiologically acceptable composition described herein in a method of treating individuals with gestational diabetes. Gestational diabetes refers to the development of diabetes in an individual during pregnancy, usually during the second or third trimester of pregnancy.

In further preferred embodiments, the pharmaceutical or physiologically acceptable composition described herein may be used in a method for treating individuals with impaired fasting glucose (IFG). Impaired fasting glucose (IFG) is a condition in which fasting plasma glucose levels in an individual are elevated but not diagnostic of overt diabetes, i.e. plasma glucose levels of less than 126 mg/dl and greater than or equal to 110 mg/dl.

In further preferred embodiments, the pharmaceutical or physiologically acceptable composition described herein may be used in a method for treating and preventing impaired glucose tolerance (IGT) in an individual. By providing therapeutics and methods for reducing or preventing IGT, i.e., for normalizing insulin resistance, the progression to NIDDM can be delayed or prevented. Furthermore, by providing therapeutics and methods for reducing or preventing insulin resistance, the invention provides methods for reducing and/or preventing the appearance of Insulin-Resistance Syndrome.

In further preferred embodiments, the pharmaceutical or physiologically acceptable composition described herein may be used in a method for treating a subject having polycystic ovary syndrome (PCOS). PCOS is among the most common disorders of premenopausal women, affecting 5-10% of this population. Insulin-sensitizing agents, e.g., troglitazone, have been shown to be effective in PCOS and that, in particular, the defects in insulin action, insulin secretion, ovarian steroidogenosis and fibrinolysis are improved (Ehrman et al. (1997) J Clin Invest 100:1230), such as in insulin-resistant humans. Accordingly, the invention provides methods for reducing insulin resistance, normalizing blood glucose thus treating and/or preventing PCOS.

In further preferred embodiments, the pharmaceutical or physiologically acceptable composition described herein may be used in a method for treating a subject having insulin resistance.

In further preferred embodiments, a subject having insulin resistance is treated according to the methods of the invention to reduce or cure the insulin resistance. As insulin resistance is also often associated with infections and cancer, prevention or reducing insulin resistance according to the methods of the invention may prevent or reduce infections and cancer.

In further preferred embodiment, the methods of the invention are used to prevent the development of insulin resistance in a subject, e.g., those known to have an increased risk of developing insulin resistance.

Thus, any of the above-described tests or other tests known in the art can be used to determine that a subject is insulin resistant, which patient can then be treated according to the methods of the invention to reduce or cure the insulin resistance. Alternatively, the methods of the invention can also be used to prevent the development of insulin resistance in a subject, e.g., those known to have an increased risk of developing insulin-resistance.

The examples set forth below are intended to illustrate but not limit the invention.

### EXAMPLES

In the following studies a group of subjects were selected according to specific parameters relating to fat deposition. Nucleic acid samples obtained from individuals in the study group were subjected to genetic analysis, which identified associations between central obesity and certain polymorphic regions in the PLA2G1B gene on chromosome 12. Polymorphic variations identified as being associated with central obesity were further screened in subjects with NIDDM to determine if they are also associated with the development of diabetes. Methods are described for producing PLA2G1B polypeptide and PLA2G1B polypeptide variants in vitro or in vivo, PLA2G1B nucleic acids or polypeptides and variants thereof are utilized for screening test molecules for those that interact with PLA2G1B molecules. Test molecules identified as interactors with PLA2G1B molecules and PLA2G1B variants are further screened in vivo to determine whether they can reduce fat deposition or treat NIDDM.

### Example 1

### Sample Selection

In addition to simple clinical measurements, dual x-ray absorbtiometry (DEXA) was utilized to determine fat content in subjects for the genetic analysis. Central fat was the primary target variable, and data were collected using a Hologic QDR 4500 DEXA system. The central region for central fat determinations was defined as the region extending from the superior surface of the second lumbar vertebra extending inferiorly to the inferior surface of the fourth lumbar vertebra and laterally to the inner aspect of the ribcage. The amount of central fat and percent central fat was automatically calculated by the equipment and downloaded into a database.

Waist and hip measurements were generated while subjects were wearing underclothes and standing with their arms by their sides. A tape measure was utilized for these measurements, and care was taken to ensure that the tape was resting on the skin and not tight. Waist circumference was measured to the nearest centimeter at the narrowest point between the iliac crest and the lower edge of the ribs. Hip circumference was measured to the nearest centimeter at the widest point below the iliac crest.

Sample selection was restricted to female twins followed by the St. Thomas Hospital in England and the Royal North Shore Hospital in Australia. It was estimated that 552 dizygotic sibling pairs would yield statistical results of reasonable power. A further 272 unrelated individuals selected from monozygotic twin pairs were added to the sample set to increase the probability for detecting associations and also for testing gene-environment interactions. The study group was selected from this combination of dizygotic and monozygotic sibling pairs, referred to as the “selection group.”

Central fat measurements and triglyceride measurements were chosen as primary target phenotypes and twin pairs were selected from the selection group for extreme discordance and concordance. Specifically, DEXA measurements and triglyceride measurements (calorimetric enzymatic method: glycerol-3-phosphate-oxidase, peroxidase, PAP (Roche), CV %=2.6, reference range less than 2.5 mmol/L) for each individual in the selection group were arranged in ascending order, and individuals in the top and lower tenth percentile were chosen from each distribution. A small subset of individuals falling in the middle range of each distribution was chosen as a control group. In addition to primary phenotype trait information, samples for inclusion in the study group were selected based on data coverage for the following secondary phenotypes recorded by each individual: BMI, insulin resistance, high density lipoprotein in serum, waist, lipoprotein(a) in serum, insulin, hip, and waist/hip ratio.

Also, presence of diabetes, thyroid disease, and renal disease reported by each individual were primary criteria for excluding subjects from the study group. Also, insulin levels greater than 7.1 μU/ml (Microparticle Enzyme Immunoassay from Abbott Laboratories Diagnostics Division (μU/ml=pmol/L×7.175) and creatine levels greater than 160 μmol/L (measured by Jaffe method: calorimetric test in which creatine reacts with picric acid in an alkaline solution to form a yellow-red colored complex) were also used as exclusion criteria as they are indicative of these diseases. Further excluded were pairs discordant for menopausal status, twin pairs where one or both of the twins were taking lipid lowering medication, non-fasting subjects (less than eight hours eating), and twin pairs including subjects treated with beta-blockers, thiazide diuretics, or exogenous estrogen.

Selecting among dizygotic and monozygotic twins for extreme discordance or concordance for the primary phenotypes minimized complications associated with bivariate ranking. After applying exclusion criteria, 253 monozygotic subjects were available for inclusion, which fell short of the target population of 276. In reaction to this situation, the extreme 201 subjects were selected from the 253 subjects and the desired numbers were reached by adding monozygotic unrelated individuals with data for central fat only, and unrelated individuals from the dizygotic cohort with data for triglycerides only. Samples available for final selection for the 552 dizygotic pairs included 178 pairs extreme for both traits, 205 extreme for triglycerides only, and 208 for central fat only.

In this test population, coverage for the secondary phenotypes ranged from 67% to 90%. In total, 61% of subjects had coverage for all primary and secondary phenotypes. A broad age spectrum was also represented, and numbers of pre-menopausal and post-menopausal subjects were relatively evenly distributed.

### Example 2

### Association of Polymorphic Variations to Fat Deposition

Blood samples were taken from individuals in the study population described in Example 1. Genomic DNA was extracted from these blood samples using standard techniques (BACC2 DNA extraction kit (Nucleon Biosciences)) and subjected to analysis. Based upon a background linkage study and fine mapping analysis by microsatellite markers, it was postulated that genetic elements linked to central fat deposition were located on the 12q24 region of chromosome twelve. One of the genes located in this region encoded PLA2G1B.

Whole genome linkage scans were performed for the purpose of identifying genomic regions likely to harbor genes with a major contribution to deposition of central fat. The linkage scans were performed using highly polymorphic microsatellite markers (Reed et al., Nat Genet 7:390-5 (1994)) and DNA samples obtained from 1100 Caucasian female twin pairs from the UK. Samples selected for inclusion in the study cohort encompassed a broad spectrum of phenotypic trait values, ranging from lean to obese subjects. Initial studies were carried out using 400 commercially available microsatellite markers derived from the Genethon linkage map, with an average genomic spacing between markers of approximately 10 cM (ABI Prism linkage mapping set, version 2 from PE Applied Biosystems).

Multipoint nonparametric linkage analysis was performed using MAPMAKER/SIBS (Kruglyak & Lander, Amer. J. Human Genetics 57:439-454 (1995)). A bioinformatics infrastructure and software packages described in WO 00/51053 were used in the linkage study to record marker positions, store data and generate data files. Output from these systems was then used with relevant application software to perform the statistical analysis.

Genotyping reactions were generally carried out in microtitre plates (384-well, reaction volume 5 μl), containing 12.5 ng of DNA from study subjects was amplified using PCR and sequence specific oligonucleotide primers labeled with 6-FAM™, HEX™, or NED™ fluorescent dyes. PCR products were analyzed by electrophoresis in a polyacrylamide denaturing gel, with an ABI PRISM™ GENESCAN® 400 HD ROX labeled size standard in each lane on an ABI model 377 analyzer (Applied Biosystems, Foster City, Calif.). For genotyping, the chosen markers were divided into two groups (panels) so that the analysis of all of the markers could be performed in two electrophoresis runs of each sample. Consequently, there was no overlap of fragment sizes in any one dye for either of the panels. Genotype analysis was performed using ABI PRISM™ GENESCAN® software (version 3.0), and genotyped manually using ABI PRISM™ Genotyper 2.0. Results were input into a database and binned by marker. The results were quality checked, ensuring consistent inheritance within families. Families that were found to have consistent pedigree problems were excluded from the analysis set.

The ordering of genetic mapping markers (i.e. microsatellite markers) was relatively stable in the region analyzed according to the Unified Data Base for Human Genome Mapping, Weizmann Institute of Science (UDB) and National Center for Biotechnology Information, National Institutes of Health (NCBI) assemblies during the duration of the study. Conversion of genetic to physical positions for strategic microsatellite markers was performed using UDB and NCBI as the reference standards. Comparisons of the identity and positioning of genomic contigs in the region were also made between UDB and NCBI and provided relatively good agreement. A comparison of the positioning of all identified and predicted genes within the region was also made between NCBI (build 22) and Joint Project between European Bioinformatics Institute and the Sanger Centre (ENSEMBL).

Microsatellite marker analysis showed linkage on the long arm of chromosome 12 to central fat deposition, percent central fat and total fat in the region spanning 125 cM to 155 cM, with a peak non parametric Z score of 3.6 for central fat. The region was further narrowed to identify the chromosomal interval 12q24 as being the primary region harboring genes contributing to central fat deposition using the following highly polymorphic microsatellite markers: D12S86, D12S1612, D12S1614, D12S340, D12S324, D12S1675, D12S1679, D12S1659 and D12S97.

The chromosome 12q24 region was then analyzed using single nucleotide polymorphisms to identify genes in the region that regulate central fat deposition. Potential polymorphisms in the PLA2G1B polynucleotide were identified in a publicly available SNP database (see http address www.ncbi.nlm.nih.gov/SNP) and were verified in a group other than the study group. Polymorphisms verified as statistically significant SNPs (minor allele represented in more than 10% of the population) were genotyped in the study population to determine associations with fat deposition. A procedure for detecting polymorphisms was utilized in the verification and genotyping studies, described hereafter. Table 1 shows the majority of polymorphisms subjected to genotype analysis and allelic variability reported in dbSNP.

TABLE 1

| Reference | Position in SEQ | Reported Allelic |
|---|---|---|
| SNP ID | ID NO: 1 | Variability |
| rs2701632 | 436 | T/C |
| rs2009391 | 4050 | A/C |
| rs5631 | 4689 | T/A |
| rs5632 | 6282 | A/C |
| rs5633 | 6358 | C/T |
| rs5634 | 7256 | T/C |
| rs5635 | 7300 | A/C |
| rs5636 | 7301 | C/A |
| rs5637 | 7328 | G/A |
| rs1186217 | 8062 | C/T |
| rs1179387 | 9182 | T/G |
| rs2701629 | 11649 | C/A |
| rs2701631 | 839 | A/T |
| rs2070873 | 6653 | T/G |
| rs2066539 | 10164 | G/A |

Assays for Verifying and Genotyping SNPs

An assay utilized for determining whether a polymorphic variation was present in a nucleic acid sample involved a sequencing by synthesis procedure. DNA polymerase, ATP sulfurylase, luciferase, apyrase, luciferin, and adenosine 5′-phosphosulfate (APS) were utilized, and in the process, one dNTP was added to an extension oligonucleotide at a time and then degraded if not incorporated in the synthesized strand. Incorporation of a dNTP to the end of the extension oligonucleotide was detected by light emission.

The assay was carried out by first amplifying a region of interest in the sample by using a polymerase chain reaction (PCR) that incorporated the primers set forth in Table 2.

TABLE 2

| | | | | | |
|---|---|---|---|---|---|
| Reference | Position in | | SEQ ID | | SEQ ID |
| SNP ID | SEQ ID NO:1 | First PCR primer | NO: | Second PCR primer | NO: |

| | | | |
|---|---|---|---|
| rs2009391 | 4050 | TGC AGA GGC TCA | CAG GTG TGG TGG |
| | | ATC ACT GT | TGG ATT G |
| rs5631 | 4689 | CAC AGG CCA CAG | TCA GAC TTG CAG |
| | | CAA ACA G | GTT GAA AAA G |
| rs5632 | 6282 | GGC AGA CCG ATT | CGG GAT CAC GCA |
| | | TGA ACT CT | CTT GA |
| rs5633 | 6358 | GGC AGT TCC GCA | TGC AGG CGG ATC |
| | | AAA TGA T | ACT TAC TT |
| rs5634 | 7256 | AGC TGT CCC TCC | GTG TGG GTG TAC |
| | | CAC TTT C | GGG TTG T |
| rs5635 | 7300 | AGC TGT CCC TCC | ATA GGT CAA GGA |
| | | CAC TTT C | AGG GAT AAA C |
| rs5636 | 7301 | AGC TGT CCC TCC | ATA GGT CAA GGA |
| | | CAC TTT C | AGG GAT AAA C |
| rs5637 | 7328 | CAA GAA GCT GGA | ATA GGT CAA GGA |
| | | CAG CTG TA | AGG GAT AAA C |
| rs1186217 | 8062 | ATC ACC TCA ACC | GGT GGT GCA CGC |
| | | TCC GTT CA | TTG TAA TT |
| rs1179387 | 9182 | AAG GTA AGC AGA | GGT TAT GTT TGG |
| | | GAT ACG TAA ATT | GTA GTA GGA TTA |
| | | AT | TA |

A typical PCR reaction included 14.24 μl of water, 2.23 μl of PCR buffer, 1.38 μl of 1.5 mM MgCl₂, 1.12 μl of 0.125 mM dNTPs, 0.45 μl of the forward primer at a 0.2 μM concentration, 0.45 μl of the reverse primer at a 0.2 μM concentration, 0.13 μl of Taq polymerase (0.003 U/μl), and 2.3 μl of DNA sample at a 0.2 ng/μl concentration, for a total volume of 22.3 μl. The PCR reaction was normally carried out using one step at 95° C. for 10 minutes; 45 cycles at 95° C. for 30 seconds, 60° C. for 45 seconds, and 72° C. for 45 seconds; one step at 72° C. for 5 minutes; and then finalizing the reaction at 22° C.

After the PCR reaction was completed, an extension oligonucleotide was hybridized to the PCR product. Extension oligonucleotides are reported in Table 3.

TABLE 3

| Position in | |
|---|---|
| SEQ ID NO:1 | Extension Oligonucleotide |
| 4050 | TGA GAT GGG AGG ATC T (antisense) |
| 4689 | ACT GGG AAC CTC GA (antisense) |
| 6282 | GCT GAT GCC GCT G (antisense) |
| 6358 | GGA GTG ACC CCT T |
| 7256 | ACA CAT GAC AAC TGC TA |
| 7300 | GGT GTG GGT GTA CGG (antisense) |
| 7301 | GGT GTG GGT GTA CGG (antisense) |
| 7328 | CCA CAC CTA TTC ATA CTC |
| 8062 | CTT AGG CAG GAG AAT C (antisense) |
| 9182 | GTA ATG CAA CTT CAA AC |

The extension oligonucleotide was complementary to the amplified target up to but not including the polymorphism (except for examination of polymorphic sites rs2009391 and rs5635, where the extension oligonucleotide terminated one base pair to the polymorphic position), and was enzymatically extended one or a few bases through the polymorphic site. In the extension phase of the assay, a single dNTP was added to the reaction, and pyrophosphate was generated if the dNTP was added to the extension oligonucleotide. ATP sulfurylase present in the reaction mixture utilized the pyrophosphate in conjunction with APS to generate ATP. ATP drove the luciferase-catalyzed conversion of luciferin to oxyluciferin, which generated the release of visible light measured by a CCD camera. A graphic representation was generated showing a peak corresponding to the amount of light emitted, where the light was proportional to the amount of nucleotide incorporated into the extension oligonucleotide. dATP was not added to the reaction, and instead, was replaced by dATPγS, which was not turned over by luciferase. Apyrase was added to the reaction to degrade unincorporated dNTP and ATP sulfurylase-generated ATP, and when the apyrase reaction was complete, another dNTP was optionally added to the reaction for another extension phase.

An alternative assay involved a MassARRAY™ system (Sequenom, Inc.), which was utilized to perform SNP genotyping in a high-throughput fashion. This genotyping platform was complemented by a homogeneous, single-tube assay method (hME™ or homogeneous MassEXTEND™ (Sequenom, Inc.)) in which two genotyping primers anneal to and amplify a genomic target surrounding a polymorphic site of interest. A third primer (the MassEXTEND™ primer), which is complementary to the amplified target up to but not including the polymorphism, was then enzymatically extended one or a few bases through the polymorphic site and then terminated.

For each polymorphism, SpectroDESIGNER™ software (Sequenom, Inc.) was used to generate a set of PCR primers and a MassEXTEND™ primer was used to genotype the polymorphism. Table 4 shows PCR primers and Table 5 shows extension primers used for analyzing polymorphisms. The initial PCR amplification reaction was performed in a 5 μl total volume containing 1×PCR buffer with 1.5 mM MgCl₂ (Qiagen), 200 μM each of dATP, dGTP, dCTP, dTTP (Gibco-BRL), 2.5 ng of genomic DNA, 0.1 units of HotStar DNA polymerase (Qiagen), and 200 nM each of forward and reverse PCR primers specific for the polymorphic region of interest.

TABLE 4

| | | | | | |
|---|---|---|---|---|---|
| Reference | Position in | | SEQ ID | | SEQ ID |
| SNP ID | SEQ ID NO:1 | First PCR primer | NO: | Second PCR primer | NO: |

| | | | |
|---|---|---|---|
| rs2701632 | 436 | ACCCACTTAGCATCCT | TCTTATGTGGGTTCC |
| | | TCAG | TTGGG |
| rs2701631 | 839 | TGTGGCCATTGTGACT | GCCCGGGTGACAGA |
| | | GAGA | GTG |
| rs5633 | 6358 | TGTGGCAGTTCCGCAA | AGTAGCAGCCGTAGT |
| | | AATG | TGTTG |
| rs2070873 | 6653 | ACCCCGTTAGAGATGG | CTGTTGCTACATTCT |
| | | AAAC | GCCAC |
| rs5637 | 7328 | AATTTCTGCTGGACAA | CCTACTGCTACAGGT |
| | | CCCG | GATTG |
| rs1179387 | 9182 | CAAGCCAAAAGTAATG | GGATTATAGATGCCT |
| | | CAAC | TCCAC |
| rs2066539 | 10164 | TCATCTCACACTGTAC | CAATATCCAAACATG |
| | | TCTC | AGGTC |
| rs2701629 | 11649 | GACAGAGAGAGACAC | GAAATGCAAGCTGTT |
| | | TATCT | ATTGG |

Samples were incubated at 95° C. for 15 minutes, followed by 45 cycles of 95° C. for 20 seconds, 56° C. for 30 seconds, and 72° C. for 1 minute, finishing with a 3 minute final extension at 72° C. Following amplification, shrimp alkaline phosphatase (SAP) (0.3 units in a 2 μl volume) (Amersham Pharmacia) was added to each reaction (total reaction volume was 7 μl) to remove any residual dNTPs that were not consumed in the PCR step. Samples were incubated for 20 minutes at 37° C., followed by 5 minutes at 85° C. to denature the SAP.

Once the SAP reaction was complete, a primer extension reaction was initiated by adding a polymorphism-specific MassEXTEND™ primer cocktail to each sample. Each MassEXTEND™ cocktail included a specific combination of dideoxynucleotides (ddNTPs) and deoxynucleotides (dNTPs) used to distinguish polymorphic alleles from one another. In Table 5, ddNTPs are shown and the fourth nucleotide not shown is the dNTP (e.g., in the first row A, C and G are ddNTPs and T is the dNTP).

TABLE 5

| | | | |
|---|---|---|---|
| | | | Termin- |
| Position in | | SEQ ID | ation |
| SEQ ID NO:1 | Extend Probe | NO: | Mix |

| | | |
|---|---|---|
| 436 | TTAGCATCCTTCAGGCCTAAA | A,C,G |
| 839 | GACTCTGCCTCAAAATAAATAAAA | C,G,T |
| | (antisense) | |
| 6358 | GCCGTAGTTGTTGTATTCCAA | A,C,T |
| | (antisense) | |
| 6653 | GTGCAAAACAGTGGGCGATGCT | A,C,T |
| 7328 | TGATTGCCGAGCCAGAGCA | A,C,G |
| | (antisense) | |
| 9182 | TTTCCATAATAGATATTTATGTAG | C,G,T |
| | (antisense) | |
| 10164 | CACTGTACTCTCCAATAAAGCACC | A,C,G |
| 11649 | CAAACAAACACACACACAAAAC | C,G,T |

The MassEXTEND™ reaction was performed in a total volume of 9 μl, with the addition of 1× ThermoSequenase buffer, 0.576 units of ThermoSequenase (Amersham Pharmacia), 600 nM MassEXTEND™ primer, 2 mM of ddATP and/or ddCTP and/or ddGTP and/or ddTTP, and 2 mM of dATP or dCTP or dGTP or dTTP. The deoxy nucleotide (dNTP) used in the assay normally was complementary to the nucleotide at the polymorphic site in the amplicon. Samples were incubated at 94° C. for 2 minutes, followed by 55 cycles of 5 seconds at 94° C., 5 seconds at 52° C., and 5 seconds at 72° C.

Following incubation, samples were desalted by adding 16 μl of water (total reaction volume was 25 μl), 3 mg of SpectroCLEAN™ sample cleaning beads (Sequenom, Inc.) and allowed to incubate for 3 minutes with rotation. Samples were then robotically dispensed using a piezoelectric dispensing device (SpectroJET™ (Sequenom, Inc.)) onto either 96-spot or 384-spot silicon chips containing a matrix that crystallized each sample (SpectroCHIP™ (Sequenom, Inc.)). Subsequently, MALDI-TOF mass spectrometry (Biflex and Autoflex MALDI-TOF mass spectrometers (Bruker Daltonics) can be used) and SpectroTYPER™ software (Sequenom, Inc.) were used to analyze and interpret the SNP genotype for each sample.

SNP Verification

Polymorphisms identified in the publicly available database were verified by detecting the presence or absence of each polymorphism across six individuals from Sweden (including PCR negative control and one sequence primer extension control). Where a polymorphism was present in two or more of the individuals, the polymorphism was designated as a statistically significant SNP and genotyped across the test population. Where the polymorphism was not identified in any of the six individuals, it was further examined in a population of thirty Caucasian blood donors from Sweden. In this group of thirty individuals, a polymorphism having a frequency of 10% or greater was designated as a statistically significant SNP and genotyped across the test population. The probability of not identifying a minor allele variant represented in 10% or more of a population was calculated as being about 0.2% when sample from 30 individuals are analyzed, where it was estimated that 19% of individuals in the total population would be carriers for the minor allele assuming a large population and no selection pressure. Also, polymorphisms were verified in a group of samples isolated from 92 individuals originating from the state of Utah in the United States, Venezuela and France (Coriell cell repositories).

The following polymorphisms reported in the dbSNP database were identified as being polymorphic (i.e., statistically significant) in the verification studies: rs2701632, rs200931, rs5631, rs5632, rs5634, rs5637, rs 1186217, rs1179387, rs2701629, and rs2070873. Polymorphisms reported in the dbSNP database as rs2701631, rs2066539, rs5633, rs5635 and rs5636 were identified as not polymorphic when tested in seventeen individuals.

Genotype Analysis

Among the verified SNPs, Table 6 depicts two SNPs that were strongly associated with reduced fat deposition. Allele frequency is noted in the second column and the allele indicated in bold type is the allele associated with decreased central fat deposition. These positions were found to be in strong linkage disequilibrium (LD). Statistical significance of each association was determined by the Monks-Kaplan test using a point-wise analysis (Monks & Kaplan, Am. J. Hum. Genet. 66: 576-592 (2000)).

TABLE 6

| | | Statistical Significance |
|---|---|---|
| SNP Position in | | using Monk-Kaplan |
| SEQ ID NO: 1 | Allele Frequency | Analysis |
| 7328 | A 0.15588 | p = 0.006691 |
| | G 0.84412 | |
| 9182 | G 0.14776 | p = 0.006884 |
| | T 0.85224 | |

Correction for multiple testing was also carried out for PLA2G1B, after removal of the other SNPs from the dataset. The value obtained for multiple correction in this manner was p=0.0859.

Haplotype analysis was performed using a program known as QPDT (Martin et al., Amer. J. Human Genetics, 67: 146-54 (2000)), which utilizes the EM algorithm (Dempster et al., J. Royal Statistical Soc., B39: 1-38 (1977)). The program was utilized to assign haplotypes based on likelihood of maximization. Table 7 shows possible haplotypes for four SNPs in the PLA2G1B gene and estimated frequencies for each.

TABLE 7

Nucleotide Position
in SEQ ID NO: 1

| Allele | 4050 | 7256 | 7328 | 9182 | Frequency |
|---|---|---|---|---|---|
| H1 | G | T | G | T | 0.51297 |
| H2 | T | T | G | T | 0.29625 |
| H3 | T | T | A | G | 0.14467 |
| H4 | G | C | G | T | 0.04292 |
| H5 | G | T | A | G | 0.00108 |

Haplotype versus single position association analysis for the PLA2G1B gene suggested that the H3 haplotype and H5 haplotype were most significantly associated with leanness. These haplotypes are characterized by an A at position 7328 and a G at position 9182.

### Example 3

### NIDDM Sample Selection

Pooling Strategies

Samples were placed into one of four groups based on disease status. The four groups were female case samples, female control samples, male case samples, and male control samples. A select set of samples from each group were utilized to generate pools, and one pool was created for each group. Each individual sample in a pool was represented by an equal amount of genomic DNA. For example, where 25 ng of genomic DNA was utilized in each PCR reaction and there were 200 individuals in each pool, each individual would provide 125 pg of genomic DNA. Inclusion or exclusion of samples for a pool was based upon the following criteria and detailed in the tables below. Selection criteria for the study described herein included patient ethnicity and diagnosis with NIDDM. Other phenotypic data collected included GAD antibody concentration, HbA1c concentration, body mass (BMI), patient age, date of primary diagnosis, age of individual as of primary diagnosis (See Table 8 below). Cases with elevated GAD antibody titers and low age of diagnosis were excluded to increase the homogeneity of the diabetes sample in terms of underlying pathogenesis. Controls with elevated HbA1c were excluded to remove any undiagnosed diabetics. Control samples were derived from non-diabetic individuals with no family history of NIDDM. Secondary phenotypes were also measured in the diabetic cases, phenotypes such as HDL, LDL, triglycerides, insulin, C-peptide, nephropathy status, neuropathy status, to name a few, which will allow secondary analysis of the cases the be performed in order to elucidate the potential pathway of the disease gene.

TABLE 8

| | | Actual no. | |
|---|---|---|---|
| | No. of individuals | of samples | No. of |
| | fulfilling | excluded | samples |
| Exclusion Criteria | exclusion criteria | after each stage | remaining |
| ALL SAMPLES Lack | 34 | 34 | 1591 |
| of availability of sample | | | |
| ALL SAMPLES | 261 | 239 | 1352 |
| Non-German ethnicity | | | |
| CASES GAD Ab > 0.9 | 102 | 84 | 1268 |
| CONTROLS HbA1c ≧ | 21 | 20 | 1248 |
| 6 or BMI >40 | | | |
| CASES age <90 | 17 | 6 | 1242 |
| CASES Age of | 150 | 203 | 1039 |
| Diagnosis <35, | | | |
| CONTROLS Family | 170 | | |
| History of Diabetes | | | |
| CONTROLS | 43 | 43 | 996 |
| Age-matching | | | |
| to case pool | | | |

The selection process yielded the pools set forth in Table 9, which were used in the studies described herein.

TABLE 9

| | Female | Female | Male | Male |
|---|---|---|---|---|
| | case | control | cases | control |
| Pool size | 244 | 244 | 254 | 254 |
| (Number) | | | | |
| Pool Criteria | case | control | case | control |
| (ex: case/control) | | | | |
| Mean Age | 52.49 | 49.02 | 49.78 | 50.57 |
| (ex: years) | | | | |

### Example 4

### Association of Polymorphic Variations with NIDDM

Blood samples were taken from individuals in the study population described in Example 3. Genomic DNA was extracted from these blood samples using standard techniques (BACC2 DNA extraction kit (Nucleon Biosciences)) and subjected to analysis. Based upon the the coexistence of all of the following or differing combinations of central fat, hypertension, glucose intolerance, dyslipidemia (elevated triglycerides and low HDL cholesterol), and impaired insulin stimulated glucose uptake (“insulin resistance”) in a common disorder referred to as syndrome X, it was postulated that polymorphic variants associated with the development of cental obesity would also be associated with NIDDM.

The SNP at position 7256 of SEQ ID NO: 1 was also allelotyped and genotyped in NIDDM and non-NIDDM patients from the pool described above (see Example 4). The following PCR primers were used: ACGTTGGATGGGGTTGTCCAGCAGAAATTTAC (forward PCR primer) and ACGTTGGATGCTTTCCAGGTGCTGCCAG (reverse PCR primer); and AGACACATGACAACTGCTA (extend primer).

Genotype Analysis

The SNP at position 7256 of SEQ ID NO: 1 was allelotyped and genotyped in NIDDM and non-NIDDM patients as described in Example 2. Table 10 shows the allelotyping results for the SNP at position 7256. Allele frequency is noted in the second column and the allele indicated in bold type is the allele associated with NIDDM. Table 11 shows the genotyping results for the SNP at position 7256. Genotype frequency in cases and controls is noted in columns 2, 3 and 4. Statistical significance of each association was determined by the Pearson Chi-squared test.

TABLE 10

| SNP Position 7256 | | | |
|---|---|---|---|
| in | Allele Frequency | Allele Frequency | Statistical |
| SEQ ID NO: 1 | Cases | Controls | Significance |
| Females | T = 0.924 | T = 0.934 | p = 0.736 |
| | C = 0.076 | C = 0.066 | |
| Males | T = 0.895 | T = 0.946 | p = 0.048 |
| | C = 0.105 | C = 0.054 | |

TABLE 11

| SNP Position 7256 | | | | |
|---|---|---|---|---|
| in | | | | Statistical |
| SEQ ID NO: 1 | TT | TC | CC | Significance |
| Case Female | 0.886 | 0.114 | 0.000 | p = 0.461 |
| Control Female | 0.901 | 0.094 | 0.005 | |
| Case Male | 0.915 | 0.077 | 0.008 | p = 0.022 |
| Control Male | 0.855 | 0.145 | 0.000 | |

Both allelotyping and genotyping analysis revealed that a cytosine at position 7256 of SEQ ID NO: 1 is associated with NIDDM (most significantly in males). Interestingly, a guanine at position 7328 and a thymine at position 9182 of SEQ ID NO: 1 were found to be associated with central obesity (see Example 2). Therefore, the data demonstrates these SNP serve as a marker for an increased risk of developing obesity or diabetes either separately or together as part of a greater metabolic syndrome.

### Example 5

### PLA2G1B Tissue Expression Profiles

PLA2G1B expression levels were determined in tissues of Israeli sand rats (Psamommys obesus) by detecting RNA transcribed from the PLA2G1B gene. P. obesus is a polygenic animal model ideal for the study of obesity and type 2 diabetes. P. obesus displays a range of pathophysiologic phenotypic responses when fed a standard laboratory diet ad libitum and animals were classified into four groups as set forth in Table 12.

TABLE 12

| Group | Phenotype | Plasma glucose/Insulin |
|---|---|---|
| Group A | Healthy | Normoglycemic/normoinsulinemic |
| Group B | Insulin resistant | Normoglycemic/hyperinsulinemic |
| Group C | Diabetic/Obese | Hyperglycemic/hyperinsulinemic |
| Group D | Diabetic/Obese | Hyperglycemic/hypoinsulinemic |

Studies were typically performed on group A, B and C animals as group D animals developed decompensated diabetes when their pancreas failed, leading to rapid death. Animals were classified at 16 weeks age following body weight, blood glucose and plasma insulin measurements at 8, 12 and 16 weeks. Body weight, blood glucose, and blood insulin were measured in grams, mmol/L, and mμ/L, respectively. Animals were considered lean at 12 weeks if their body weight was less than 180 grams and obese when body weight was greater than 200 grams. Animals were considered normoglycemic and normoinsulinemic if their blood glucose levels were less than 8.0 mmol/L and insulin levels were less than 150 mμ/L. Animals were classified as hyperinsulinemic if their blood insulin levels were equal to or greater than 150 mμ/L. Animals were further classified as diabetic if their blood glucose levels were equal to or greater than 8 mmol/L.

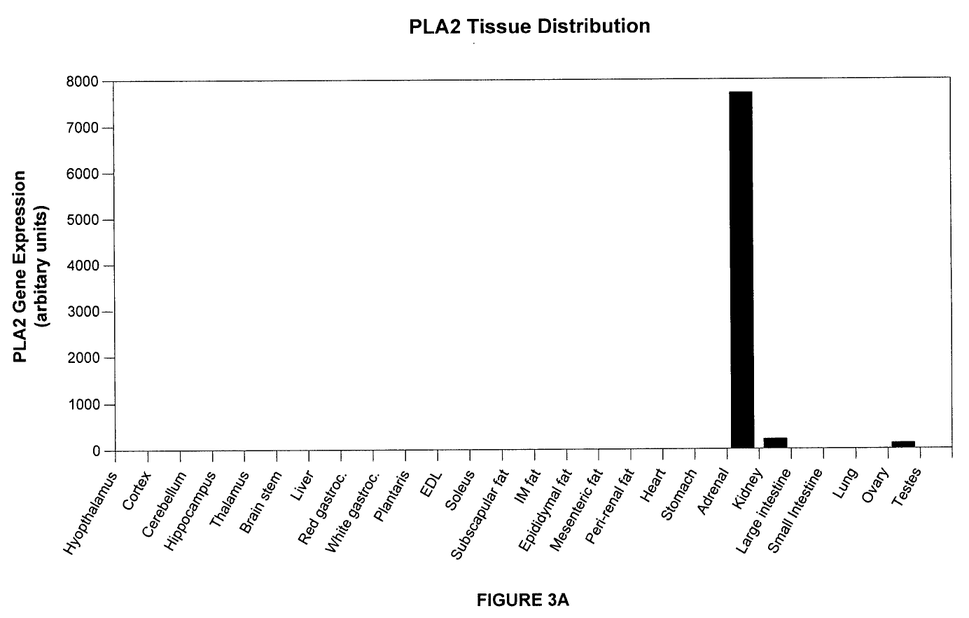

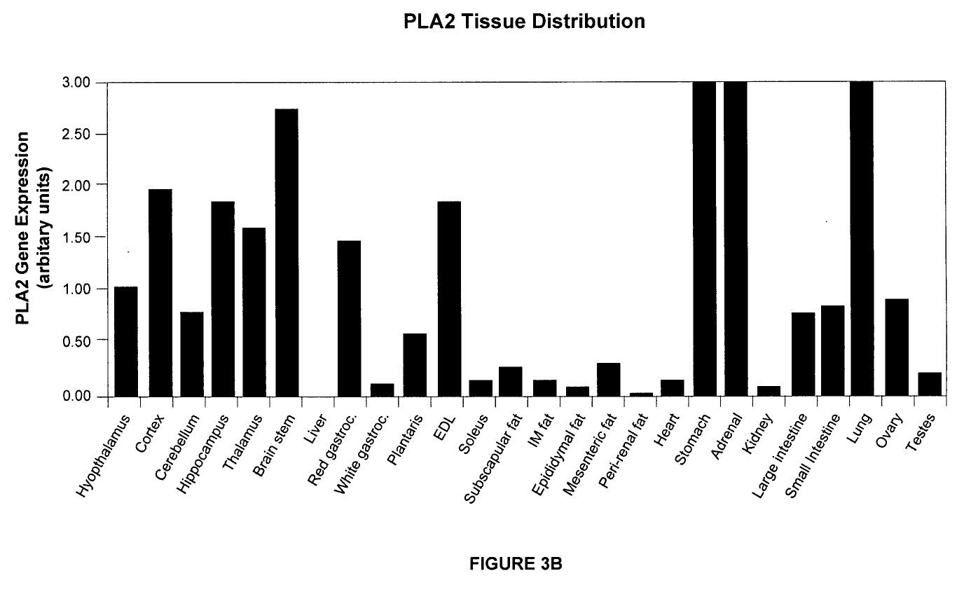

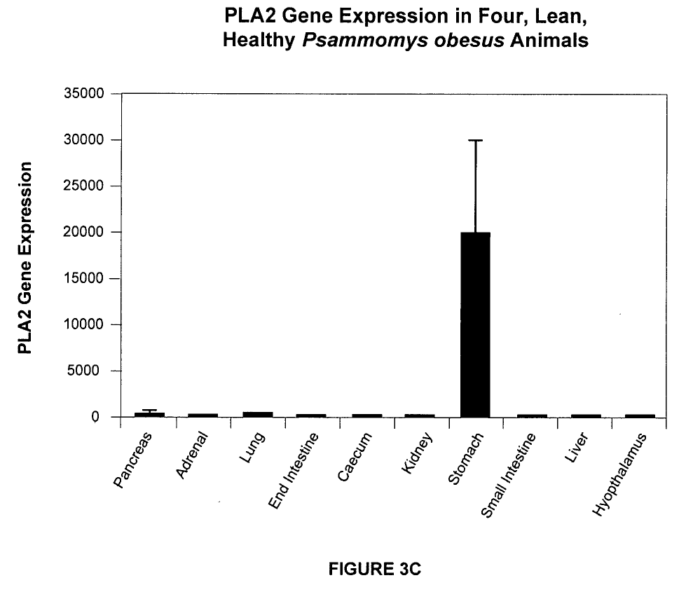

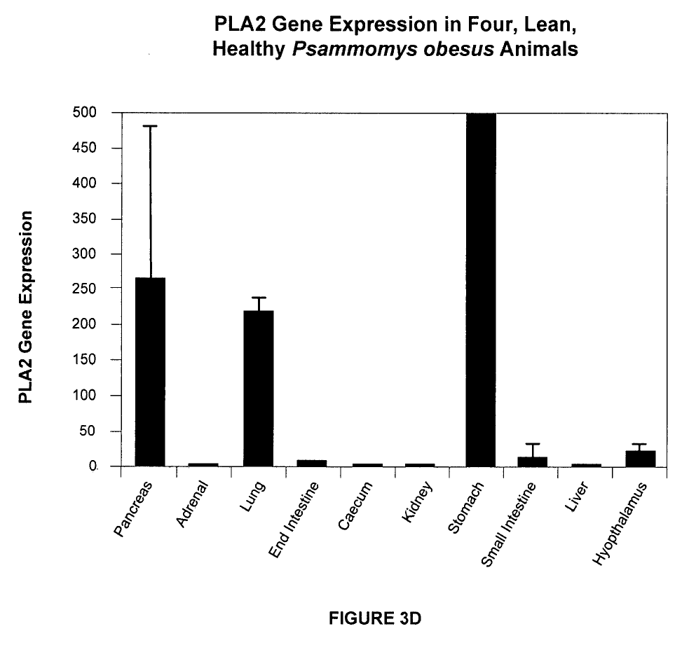

PLA2G1B tissue distribution expression profiles were studied in male P. obesus group A animals (lean and healthy) and the results are depicted in FIGS. 3A-3D. Animals were normally fasted for two hours prior to tissue harvesting. As shown in FIGS. 3A-3D, PLA2G1B expression was highest in stomach tissue, and expressed at lower levels in pancreatic, lung, and adrenal tissue. Expression was also observed in the large and small intestine.

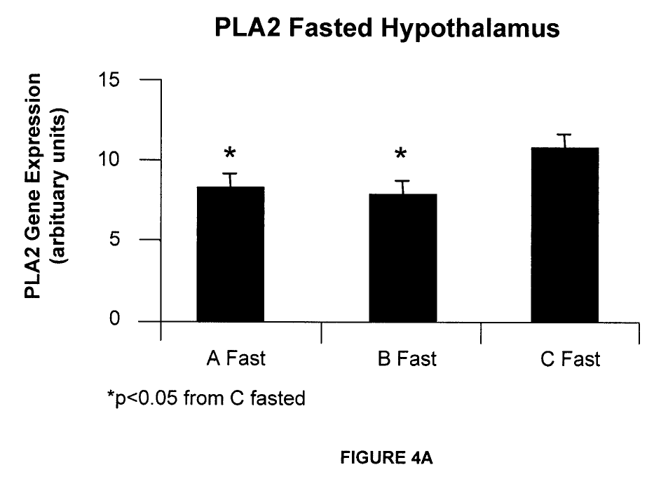

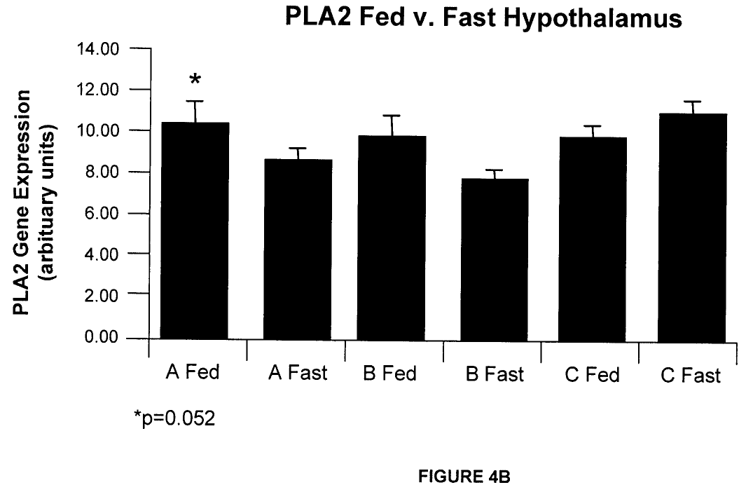

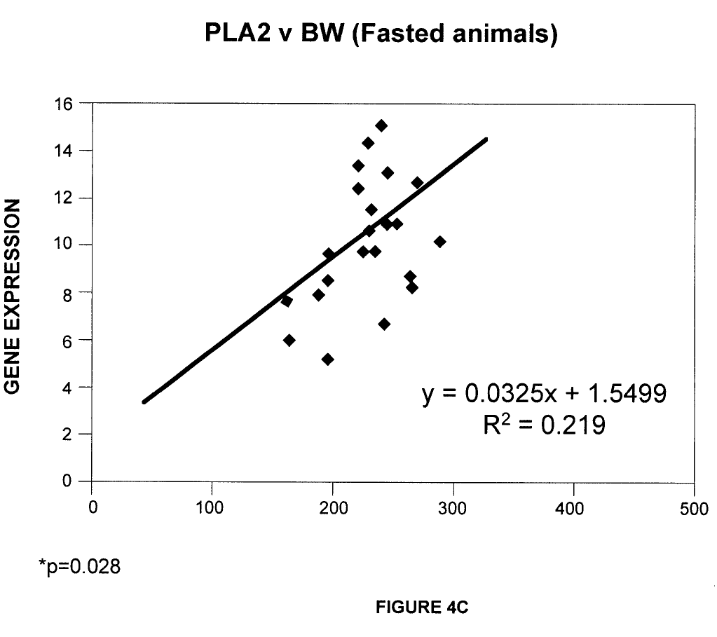

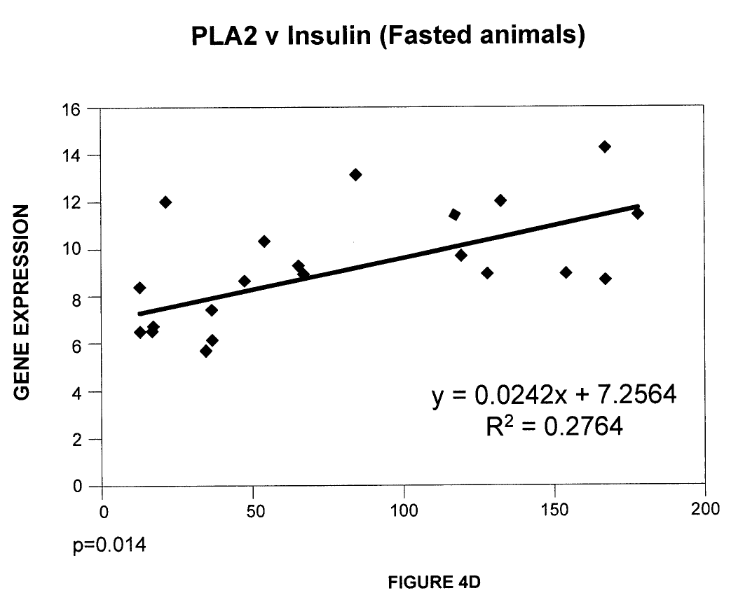

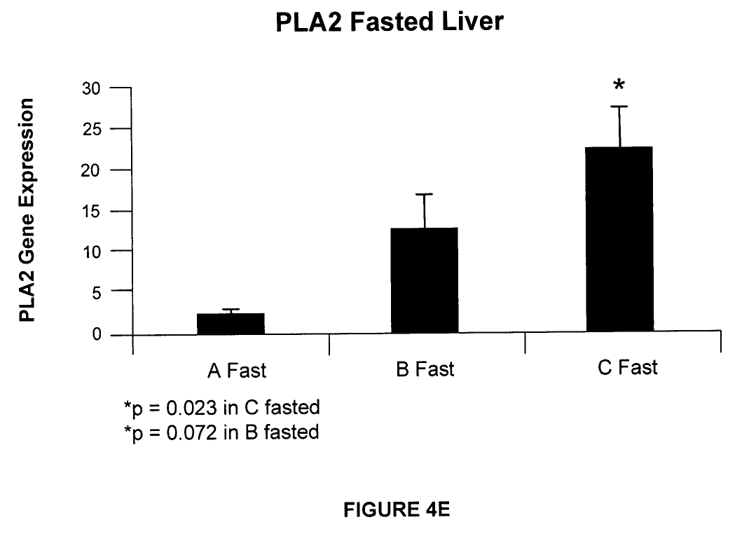

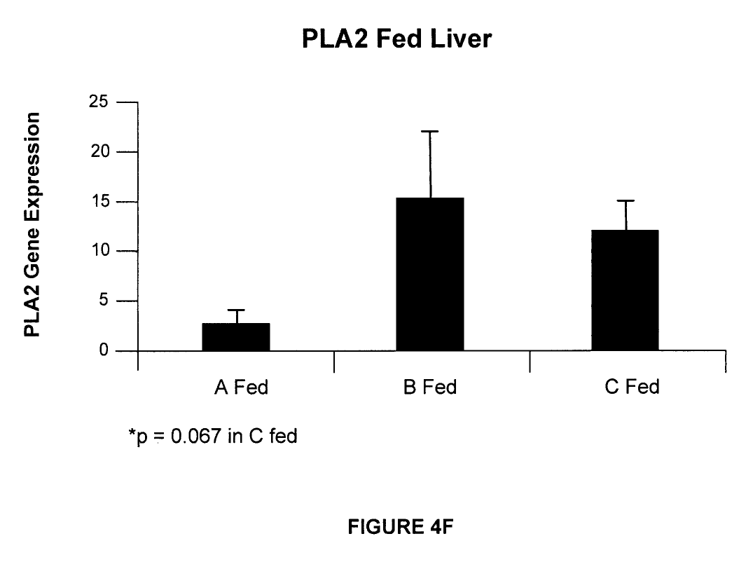

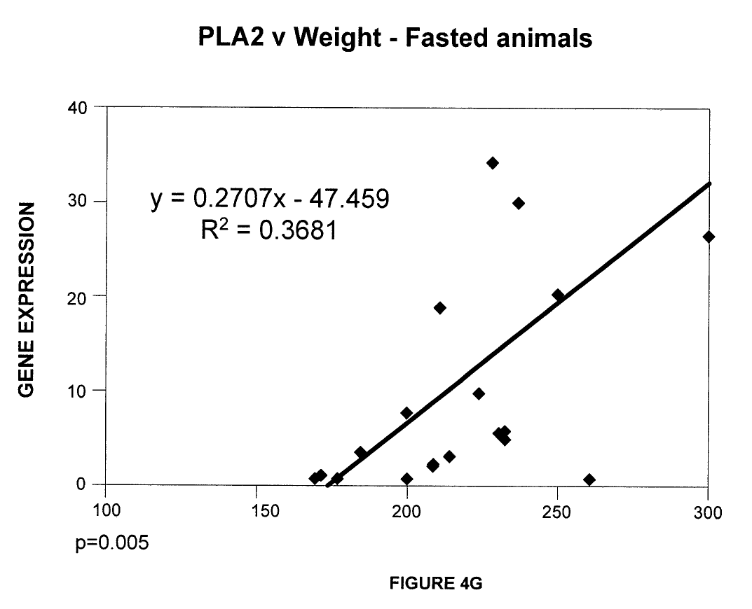

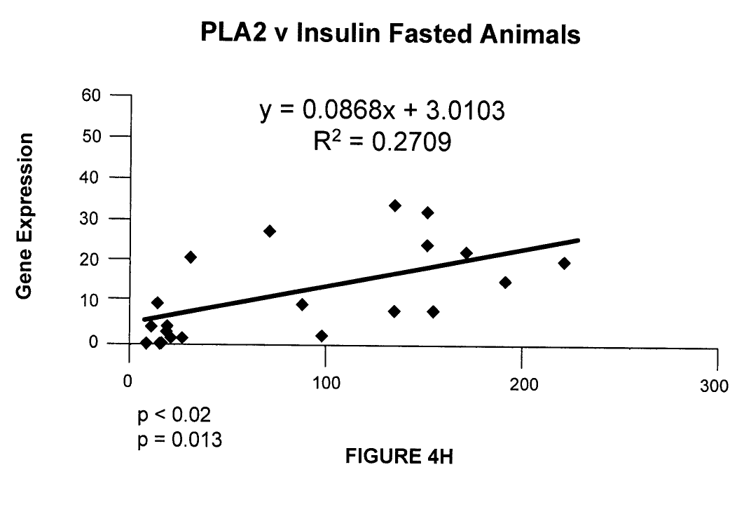

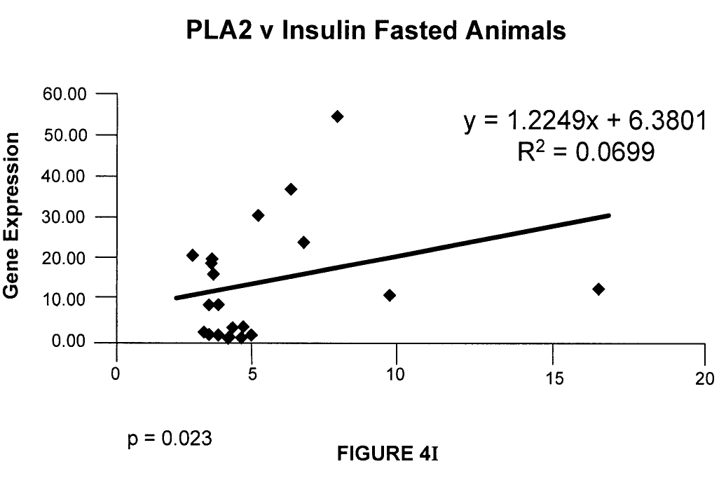

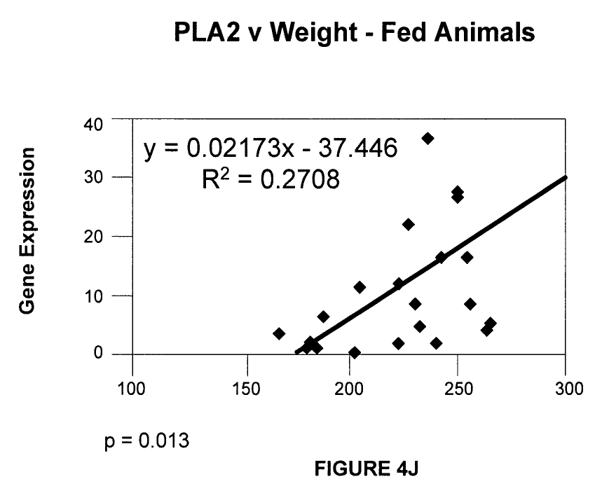

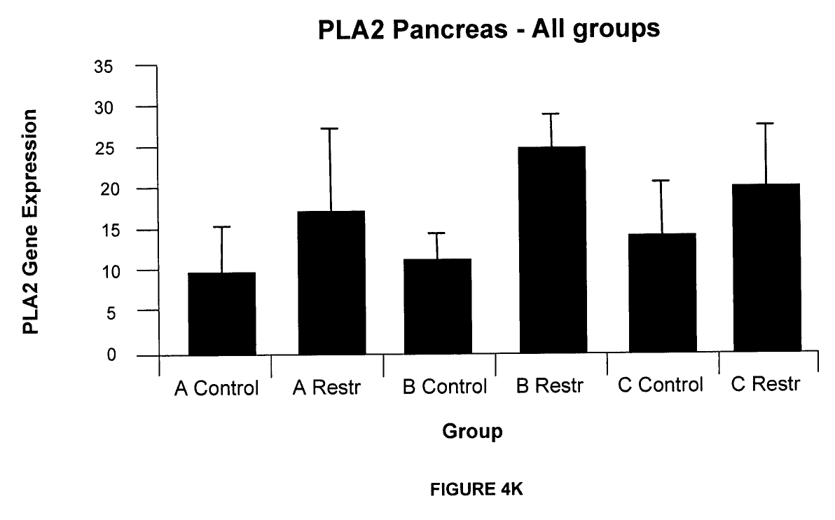

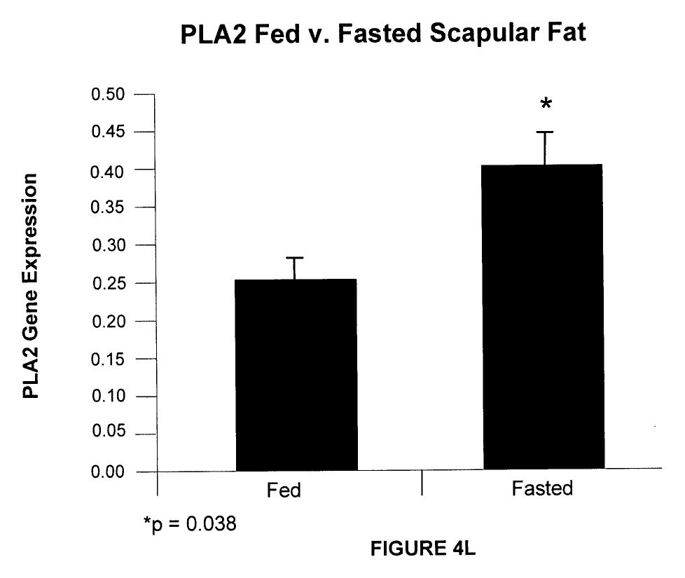

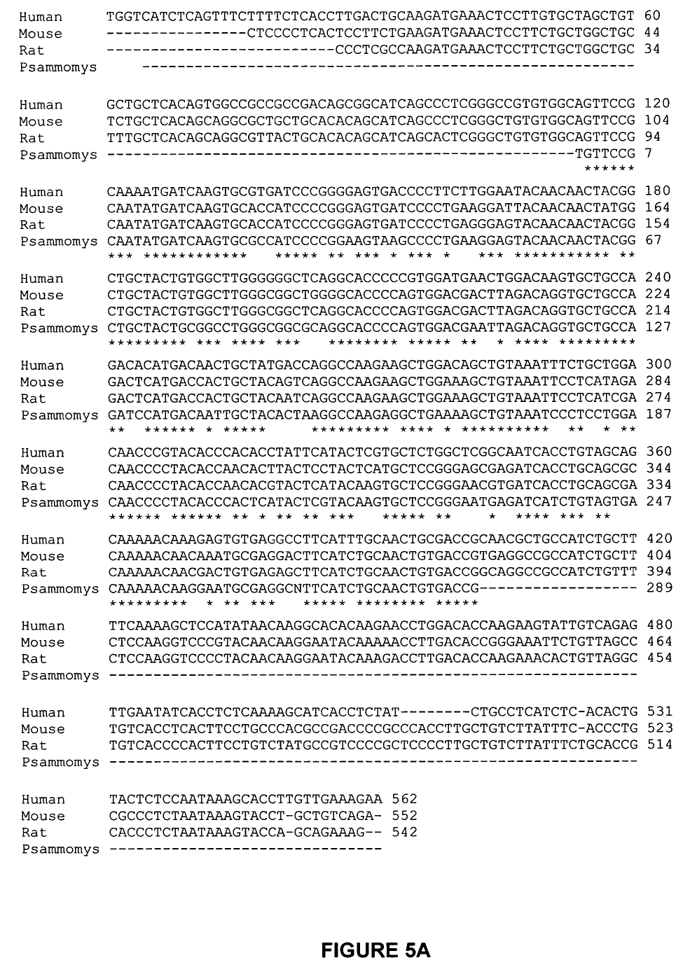

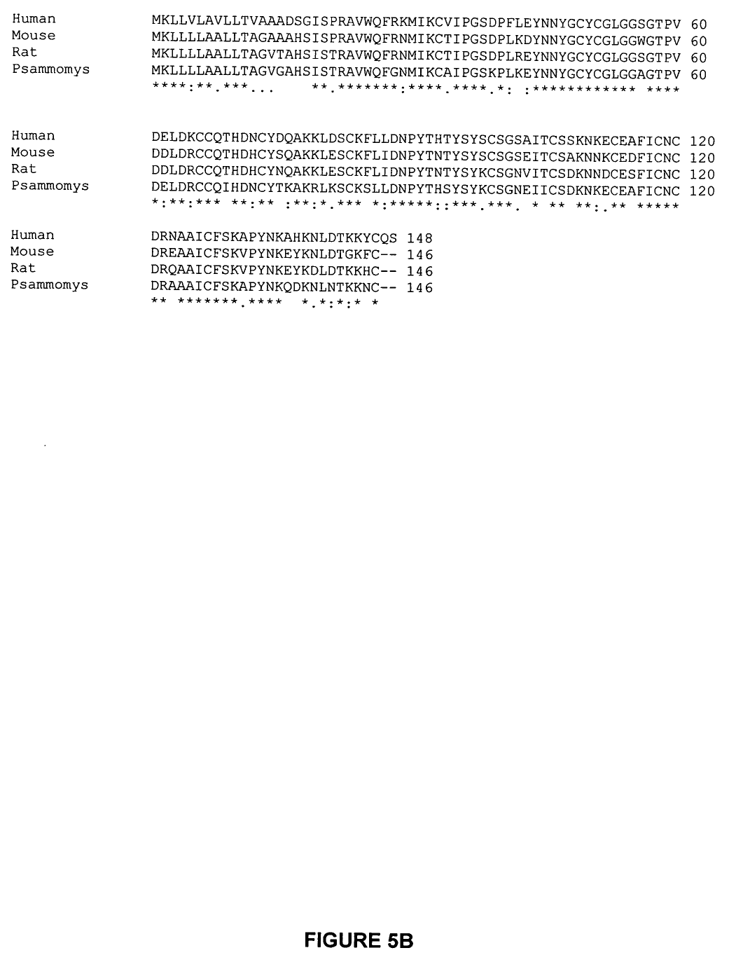

Metabolically-linked tissues, such as liver, fat pads, skeletal muscle, hypothalamus, pancreas, and stomach tissues, were targeted for analysis of differential gene expression of PLA2G1B following normal feeding or overnight fasting conditions. In addition, data relating to blood glucose, plasma insulin, body weight, and body fat from the animals were correlated against gene expression using t-test analysis. From these studies, it was determined that PLA2G1B expression in the hypothalamus was significantly greater in group C fasted animals as compared to group A fasted animals (p=0.033) and group B fasted animals (p=0.02) using a parametric t-test. (See FIG. 4A). Also, hypothalamus PLA2G1B expression in group A animals that were fed normally was greater than in fasted group A animals (p=0.052). (See FIG. 4B). In addition, hypothalamus PLA2G1B expression in fasted animals was positively associated with body weight (p=0.028) and plasma insulin levels (p=0.014) using a parametric Pearson comparison. (See FIGS. 4C and 4D).

In the liver, PLA2G1B expression in group A fasted animals tended to be lower than group B fasted animals (p=0.072) and group C fasted animals (p=0.023) using a Games-Howell parametric method of multiple comparisons. (See FIG. 4E). In addition, liver PLA2G1B expression in normally fed group A animals were lower than normally fed group C animals (p=0.067). (See FIG. 4F) Also, there were positive associations between liver PLA2G1B expression in fasted animals with body weight (p=0.005), blood insulin (p=0.013 both parametric correlations), and blood glucose (p=0.023, nonparametric correlation). (See FIGS. 4G, 4H and 4I). Further, there was a positive association between liver PLA2G1B expression in fed animals with body weight (p=0.013). (See FIG. 4J).

In the pancreas, a significant difference in PLA2G1B expression was observed between control and energy-restricted groups (p=0.036, t-test). (See FIG. 4K). There were no correlations between body weight, blood glucose, and blood insulin with pancreatic PLA2G1B expression.

In subscapular fat, PLA2G1B expression in the fasted animals was significantly greater than normally fed animals (p=0.038 t-test). (See FIG. 4L). In red gastrocnemius muscle, kidney, and stomach tissues, however, there were no significant differences in PLA2G1B expression between fed groups and between fasted groups, and no correlations between body weight, blood glucose, and blood insulin with PLA2G1B expression.

Gene expression was quantified using a TaqMan™ PCR system (ABI Prism™ 7700 Sequence Detection System, Perkin-Elmer Applied Biosystems, Norwalk, USA) and was determined relative to an endogenous control gene, cyclophilin. cDNA was synthesized by subjecting one microgram of total RNA to a reverse transcription reaction using SuperScript II RNase H-Reverse Transcriptase (Invitrogen) according to manufacturer's instructions (see http address www.invitrogen.com/Content/World/11904018.pdf). In this reverse transcriptase PCR (RT-PCR) procedure, the following contents were added to a nuclease-free microcentrifuge tube: 1 μl Oligo (dT)12-18 (500 μg/ml); 1 μg total RNA; 1 μl 10 mM dNTP mix (10 mM each dATP, dGTP, dCTP and dTTP at neutral pH); sterile, distilled water to 12 μl. The mixture was heated to 65° C. for 5 minutes and quickly chilled on ice for at least 2 minutes.

Contents of the tube were collected by brief centrifugation and the following were added to complete a 20-μl reaction volume: 2 μl 10× First-Strand Buffer; 4 μl 25 mM MgCl₂; 2 μl 0.1 M DTT; 1 μl RNaseOUT Recombinant Ribonuclease Inhibitor (40 units/μl); 1 μl (200 units) of and SUPERSCRIPT II. The mixture was incubated at 45° C. for 50 minutes and then the reaction was inactivated by heating at 70° C. for 15 minutes. To remove RNA complementary to the cDNA, 1 μl (2 units) of E. coli RNase H was added and incubated at 37° C. for 20 minutes. The resulting mixtures were transferred to 0.5 ml tubes and stored at −20° C.

Oligonucleotide primers were designed based upon the P. obesus sequence using Primer Express software (version 1.5), which was obtained at the http address docs.appliedbiosystems.com/pebiodocs/04303014.pdf. For PCR reactions, forward primers having the sequences GCTGTGTGGCAGTTCCGCAA; GTTCCGCAATATGATCAAGTGC; GATGAAACTCCTTCTGCTGGCTG; and SAAGATGAAACTCCTTCTGCTG were utilized in conjunction with reverse primers having the sequences GGTGAAATAAGACAGCAAGG; GGAGAANCAGATGGCGGCCT; CGGTCACAGTTGCAGATGAAG; GGAAGTGGGGTGACAGCCTAACA; and GGTGACAGSCTAACAGWNTTTC, where S is G or C; N is C, G, T, or A; and W is A or T. Also, another forward primer having the sequence 5′-GCACCCCAGTGGACGAATT-3′ and a reverse primer having the sequence 5′-TCAGCCTCTTGGCCTTAGTGTAG-3′ yielded an amplicon that was 70 base pairs in length and were used for RT-PCR. Primers for the endogenous control gene, cyclophilin, were designed based on the P. obesus sequence. Primer sequence specificity was confirmed by comparing the primer sequences against the GenBank nucleotide sequence for PLA2G1B using BLAST. Primers were synthesized at a 40 nmole concentration and purified by using a reverse-phase cartridge (GeneWorks, Australia).

The ability of the primers to operate in a quantitative PCR process was next determined. A standard curve was generated based upon threshold cycles (Ct=threshold cycle) for serially diluted samples. cDNA was serially diluted from a 1:2 dilution to a 1:16 dilution, and the standard curve included an undiluted sample and a “no template control” (contains no cDNA). A standard curve was also generated using primers specific for the endogenous control gene (cyclophilin). These samples are set-up in duplicate using the following: 12.5 μl of SYBR Green Universal PCR master mix (cat #4304437, http address docs.appliedbiosystems.com/pebiodocs/00777601.pdf ); 2.5 μl of forward primer (1 μM, diluted in nuclease-free water); 2.5 μl of reverse primer (1 μM, diluted in nuclease-free water); 2.0 μl of cDNA (neat or diluted); and 5.5 μl of water (nuclease-free) for a total volume of 25 μl.

The PCR program recommended for the ABI Prism 7700 procedure was utilized and the baseline was calculated based upon cycles 3 to 15 and the amplification plot was based upon cycles 16 to 40. A threshold level was set following examination of a semi-log view of the plot. The Ct values for each duplicate were examined to ensure they did not differ by more than one Ct unit. The Ct values were eliminated or the experiment was repeated if they differed by more than one Ct unit. Samples were run on an agarose gel to identify product formation and whether or not primer-dimers or non-specific priming occurred. While the primer concentration could have been optimized if required, it was determined that 100 nM of each primer (final concentration) was adequate.

Following the primer efficiency determination, a real time PCR run was executed. The conditions utilized were as described above except that cDNAs were diluted 1:8 and products were not confirmed on an agarose gel. Final values were then calculated using the relation 2^{-ΔCt}, where ΔCt is Ct of cyclophilin subtracted from Ct of the gene of interest. Gene expression values were calculated as arbitrary units, and Ct values for cyclophilin in treated samples (e.g., in fasted tissues) were further examined to determine whether endogenous control of gene expression was altered. This analysis yielded quantified and standardized gene expression values for the amount of cDNA in each reaction.

### Example 6

### In Vitro Production of PLA2G1B Polypeptides

PLA2G1B cDNA is cloned into a pIVEX 2.3-MCS vector (Roche Biochem) using a directional cloning method. A PLA2G1B cDNA insert is prepared using PCR with forward and reverse primers having 5′ restriction site tags (in frame) and 5-6 additional nucleotides in addition to 3′ gene-specific portions, the latter of which is typically about twenty to about twenty-five base pairs in length. A Sal I restriction site is introduced by the forward primer and a Sma I restriction site is introduced by the reverse primer. The ends of PLA2G1B PCR products are cut with the corresponding restriction enzymes (i.e., Sal I and Sma I) and the products are gel-purified. The pIVEX 2.3-MCS vector is linearized using the same restriction enzymes, and the fragment with the correct sized fragment is isolated by gel-purification. Purified PLA2G1B PCR product is ligated into the linearized pIVEX 2.3-MCS vector and E. coli cells transformed for plasmid amplification. The newly constructed expression vector is verified by restriction mapping and used for protein production.

E. coli lysate is reconstituted with 0.25 ml of Reconstitution Buffer, the Reaction Mix is reconstituted with 0.8 ml of Reconstitution Buffer; the Feeding Mix is reconstituted with 10.5 ml of Reconstitution Buffer; and the Energy Mix is reconstituted with 0.6 ml of Reconstitution Buffer. 0.5 ml of the Energy Mix was added to the Feeding Mix to obtain the Feeding Solution. 0.75 ml of Reaction Mix, 50 μl of Energy Mix, and 10 μg of the PLA2G1B template DNA is added to the E. coli lysate.

Using the reaction device (Roche Biochem), 1 ml of the Reaction Solution is loaded into the reaction compartment. The reaction device is turned upside-down and 10 ml of the Feeding Solution is loaded into the feeding compartment. All lids are closed and the reaction device is loaded into the RTS500 instrument. The instrument is run at 30° C. for 24 hours with a stir bar speed of 150 rpm. The pIVEX 2.3 MCS vector includes a nucleotide sequence that encodes six consecutive histidine amino acids on the C-terminal end of the PLA2G1B polypeptide for the purpose of protein purification. PLA2G1B polypeptide is purified by contacting the contents of reaction device with resin modified with Ni²⁺ ions. PLA2G1B polypeptide is eluted from the resin with a solution containing free Ni2+ ions.

### Example 7

### Cellular Production of PLA2G1B Polypeptides

PLA2G1B nucleic acids are cloned into DNA plasmids having phage recombination cites and PLA2G1B polypeptides and polypeptide variants are expressed therefrom in a variety of host cells. alpha phage genomic DNA contains short sequences known as attP sites, and E. coli genomic DNA contains unique, short sequences known as attB sites. These regions share homology, allowing for integration of phage DNA into E. coli via directional, site-specific recombination using the phage protein Int and the E. coli protein IHF. Integration produces two new att sites, L and R, which flank the inserted prophage DNA. Phage excision from E. coli genomic DNA can also be accomplished using these two proteins with the addition of a second phage protein, Xis. DNA vectors have been produced where the integration/excision process is modified to allow for the directional integration or excision of a target DNA fragment into a backbone vector in a rapid in vitro reaction (Gateway™ Technology (Invitrogen, Inc.)).

A first step is to transfer the PLA2G1B nucleic acid insert into a shuttle vector that contains attL sites surrounding the negative selection gene, ccdB (e.g. pENTER vector, Invitrogen, Inc.). This transfer process is accomplished by digesting the PLA2G1B nucleic acid from a DNA vector used for sequencing, and to ligate it into the multicloning site of the shuttle vector, which will place it between the two attL sites while removing the negative selection gene ccdB. A second method is to amplify the PLA2G1B nucleic acid by the polymerase chain reaction (PCR) with primers containing attB sites. The amplified fragment then is integrated into the shuttle vector using Int and IHF. A third method is to utilize a topoisomerase-mediated process, in which the PLA2G1B nucleic acid is amplified via PCR using gene-specific primers with the 5′ upstream primer containing an additional CACC sequence (e.g., TOPO® expression kit (Invitrogen, Inc.)). In conjunction with Topoisomerase I, the PCR amplified fragment can be cloned into the shuttle vector via the attL sites in the correct orientation.

Once the PLA2G1B nucleic acid is transferred into the shuttle vector, it can be cloned into an expression vector having attR sites. Several vectors containing attR sites for expression of PLA2G1B polypeptide as a native polypeptide, N-fusion polypeptide, and C-fusion polypeptides are commercially available (e.g., pDEST (Invitrogen, Inc.)), and any vector can be converted into an expression vector for receiving a PLA2G1B nucleic acid from the shuttle vector by introducing an insert having an attR site flanked by an antibiotic resistant gene for selection using the standard methods described above. Transfer of the PLA2G1B nucleic acid from the shuttle vector is accomplished by directional recombination using Int, IHF, and Xis (LR clonase). Then the desired sequence can be transferred to an expression vector by carrying out a one hour incubation at room temperature with Int, IHF, and Xis, a ten minute incubation at 37° C. with proteinase K, transforming bacteria and allowing expression for one hour, and then plating on selective media. Generally, 90% cloning efficiency is achieved by this method. Examples of expression vectors are pDEST 14 bacterial expression vector with att7 promoter, pDEST 15 bacterial expression vector with a T7 promoter and a N-terminal GST tag, pDEST 17 bacterial vector with a T7 promoter and a N-terminal polyhistidine affinity tag, and pDEST 12.2 mammalian expression vector with a CMV promoter and neo resistance gene. These expression vectors or others like them are transformed or transfected into cells for expression of the PLA2G1B polypeptide or polypeptide variants. These expression vectors are often transfected, for example, into murine-transformed a adipocyte cell line 3T3-L1, (ATCC), human embryonic kidney cell line 293, and rat cardiomyocyte cell line H9C2.

### Example 8

### Cellular Assay for Screening PLA2G1B Interacting Fat Reduction Drug Candidates

General PLA2 assay strategies are known (Reynolds et al., Methods in Enzymology 197: 3-23 (1991)). Sensitive and practical assays include radioactive and spectrophotometric assays. An assay optionally employing chromogenic or spectrometric detection (Yu et al., Methods in Enzymology 197: 65-75 (1991)) is often utilized for determining whether test molecules interact with PLA2G1B in a high throughput format, typically with inclusion of bile acids or other anionic detergents. The assay format has been modified with minor variations to assay the non-pancreatic GIIA PLA2 from human synovial fluid in a high throughput format (Reynolds et al., Analytical Biochemistry 204:190-197 (1992)).

A similar spectrophotometric assay was developed for GIVA PLA2 (Reynolds et al. Anal. Biochem. 217:25-32 (1994)) and is utilized to determine whether a test molecule interacts with PLA2G1B. This assay is often utilized in conjunction with a microtitre plate and plate reader in a high throughput format. In the assay, PLA2 function is monitored using a ThioPC/Triton X-100 substrate solution. An appropriate volume of ThioPC in chloroform solution is evaporated to dryness under a stream of N₂. Triton X-100 (8 mM) in 2× assay buffer (160 mM HEPES, pH 7.4,300 mM NaCl, 20 mM CaCl₂, 2 mg/ml BSA) is added to the dried lipid in one-half the desired final volume to give a 2-fold concentrated substrate solution. This solution is bath-sonicated for 1 minute to loosen dried ThioPC from the walls of the vial and then probe-sonicated on ice (20 seconds on ice, 20 seconds off ice) for 3 minutes. The solution is then warmed to 40° C. and warmed glycerol equivalent to 30% of the final volume was added. The solution is then brought to the desired final volume with deionized H₂O. The final assay contains 2 mM ThioPC, 4 mM Triton® X-100 and 30% glycerol in 80 mM HEPES, pH 7.4, 150 mM NaCl, 10 mM CaCl₂ and 1 mg/ml BSA.

The substrate is then aliquotted, in 200 μl increments, into the wells of a 96-well plate and equilibrated for 5 minutes at 37° C. To initiate the reaction, 500 ng PLA2 (purified, recombinant human), in a 5 μl volume of 1× assay buffer, is added to the wells, the plate is shaken 20 seconds on high to mix, and then incubated for 60 minutes at 37° C. For controls, buffer rather than enzyme was added to some wells.
After 60 minutes, 10 μl of a 25 mM DTNB/475 mM EGTA mixture is added to all substrate containing wells to quench the reaction and initiate the color development. The DTNB/EGTA mixture is prepared just prior to use by combining equal volumes of 50 mM DTNB in 0.5M Tris, pH 7.4 and 950 mM EGTA, pH 7.2. After adding the DTNB/EGTA, the plate is once again shaken 20 seconds on high and allowed to incubate for an additional 3 minutes to give the DTNB chromophore time to fully develop prior to reading the plate. The absorbance is measured using a dual wavelength option (405 nm to 620 nm) to correct for light scattering. The results obtained with this dual wavelength option are similar to those obtained using a single wavelength (405 nm) but are more reproducible. The average absorbance of the controls is subtracted from that of the enzyme-containing wells to correct for the absorbance due to the substrate, DTNB, and EGTA. The difference in absorbance is used to calculate enzyme activity. The data was reported ± the standard deviation. Specific activity is calculated using ε₄₀₅ for DTNB of 12,800 M^{cm-1} and a path length of 0.47 cm for a 215 μl final total volume. The path length in these plates is dependent on the assay volume and was calculated by measuring the absorbance of several concentrations of bromothymol blue, where the path length equals the absorbance observed on the plate reader divided by the absorbance observed for the same solution in the spectrophotometer in 1 cm cuvettes. A short burst of activity is often observed in the first 5 minutes followed by a more linear phase from 5 to 60 minutes. Further details concerning this assay are disclosed in U.S. Pat. No.5,464,754. This assay also can be carried out using a modified phosphocholine substrate as is used when assaying cobra venom PLA2 molecules.

Further, assays described in (Yang et al., J. Neurochemistry 73:1278-1287 (1999)) readily can be applied to distinguish secreted PLA2 molecules (e.g., PLA2G1B) in tissues from other PLA2 forms.

### Example 9

### In Vivo Assay for Screening Fat Reduction Drug Candidates

Test molecules are screened for fat reduction activity by administering molecules which interact with PLA2G1B to Israeli sand rats (P. obsesus), which is an accepted in vivo model for obesity, and observing the effect of the molecule on such parameters as weight, dimensions, and/or fat content. Molecules may be administered to obese animals and/or non-obese animals. These animals are grouped into four sets (Table 8), where group D animals have high morbidity and are not typically used in studies.

The Israeli sand rat is maintained on an ad libitum diet of a standard lab chow that is high in energy. This polygenic animal displays in response to this diet a range of body weights, plasma insulin and blood glucose levels. Normally, eight controlled animals and eight treated animals are included for groups A, B and C, giving a total of 48 animals for each study.

The test molecule is delivered to the animals by intraperitoneal injection; intravenous injection; intragastrical administration, in which case twice as many animals per group should be used since the method of administration is more stressful and leads to a higher motility rate; continuous infusion using an osmotic pump; and orally ad libitum, which is the least stressful as the test molecule is added to food and the amount of consumed is measured. Often DMSO or water is used as a vehicle accompanying the test molecule and 10 μg to 1000 μg of test molecule per kilogram of the animal is typically administered.

The length of the study is typically one to seven days. During the study, several parameters are measured, including body weight (daily measurements); food intake (daily measurements); blood glucose levels (before and after the study); plasma insulin levels (before and after the study); circulating blood metabolites such as leptin, cortisol, triglycerides and free fatty acids (before and after the study); percent body fat (weighing fat pads at the end of the study); quantification of gene expression in tissues such as the pancreas, mesenteric fat, stomach and small intestine (at the end of the study); and measurements of PLA2G1B activity in tissues such as pancreas, mesenteric fact, stomach, and small intestine using methods described in Example 7 (before and/or after the study). Animals are sacrificed by anaesthetic overdose and tissues are harvested and rapidly frozen. RNA is extracted from half of each harvested tissue and PLA2G1B polypeptide extracts are sometimes generated from the other half.

Modifications may be made to the foregoing without departing from the basic aspects of the invention. Although the invention has been described in substantial detail with reference to one or more specific embodiments, those of skill in the art will recognize that changes may be made to the embodiments specifically disclosed in this application, yet these modifications and improvements are within the scope and spirit of the invention, as set forth in the claims which follow. All publications or patent documents cited in this specification are incorporated herein by reference as if each such publication or document was specifically and individually indicated to be incorporated herein by reference.

Citation of the above publications or documents is not intended as an admission that any of the foregoing is pertinent prior art, nor does it constitute any admission as to the contents or date of these publications or documents. U.S. patents and other publications referenced herein are hereby incorporated by reference.

1. A method for identifying a candidate therapeutic for fat reduction, which comprises:
(a) introducing a test molecule to a system which comprises a nucleic acid comprising a PLA2G1B nucleotide sequence selected from the group consisting of:
(i) the nucleotide sequence of SEQ ID NO:1;
(ii) a nucleotide sequence which encodes a polypeptide consisting of the amino acid sequence of SEQ ID NO:2;
(iii) a nucleotide sequence which encodes a polypeptide that is 90% identical to the amino acid sequence of SEQ ID NO:2; and
(iv) a fragment of a nucleotide sequence of (i), (ii), or (iii); or
introducing a test molecule to a system which comprises a protein encoded by a nucleotide sequence of (i), (ii), (iii), or (iv); and
(b) determining the presence or absence of an interaction between the test molecule and the nucleic acid or protein,
whereby the presence of an interaction between the test molecule and the nucleic acid or protein identifies the test molecule as a candidate therapeutic for fat reduction.

2. The method of claim 1, wherein the system is an animal.

3. The method of claim 1, wherein the system is a cell.

4. The method of claim 1, wherein the PLA2G1B nucleotide sequence comprises a guanine at position 7328, a thymine at position 9182, or a guanine at position 7328 and a thymine at position 9182.

5. A method for reducing fat deposition in a subject, which comprises administering a candidate therapeutic of claim 1 to a subject in need thereof, whereby the candidate therapeutic reduces fat deposition in the subject.

6. A method for reducing fat deposition in a subject, which comprises contacting a PLA2G1B nucleic acid with one or more cells of a subject in need thereof, wherein the PLA2G1B nucleic acid comprises a nucleotide sequence selected from the group consisting of:
(a) the nucleotide sequence of SEQ ID NO:1;
(b) a nucleotide sequence which encodes a polypeptide consisting of the amino acid sequence of SEQ ID NO:2;
(c) a nucleotide sequence which encodes a polypeptide that is 90% identical to the amino acid sequence of SEQ ID NO:2; and
(d) a fragment of a nucleotide sequence of (a), (b), or (c);
whereby contacting the one or more cells of the subject with the PLA2G1B nucleic acid reduces fat deposition.

7. A method for reducing fat deposition in a subject, which comprises contacting a PLA2G1B protein with one or more cells of a subject in need thereof, wherein the PLA2G1B protein is encoded by a PLA2G1B nucleotide sequence which comprises a polynucleotide sequence selected from the group consisting of:
(a) the polynucleotide sequence of SEQ ID NO:1;
(b) a polynucleotide sequence which encodes a polypeptide consisting of the amino acid sequence of SEQ ID NO:2;
(c) a polynucleotide sequence which encodes a polypeptide that is 90% identical to the amino acid sequence of SEQ ID NO:2; and
(d) a fragment of a polynucleotide sequence of (a), (b), or (c);
whereby contacting the one or more cells of the subject with the PLA2G1B protein reduces fat deposition.

8. A method for reducing fat deposition in a subject, which comprises:
detecting the presence or absence of a polymorphic variant associated with fat deposition in a PLA2G1B nucleotide sequence in a nucleic acid sample from a subject, wherein the PLA2G1B nucleotide sequence comprises a polynucleotide sequence selected from the group consisting of:
(a) the polynucleotide sequence of SEQ ID NO:1;
(b) a polynucleotide sequence which encodes a polypeptide consisting of the amino acid sequence of SEQ ID NO:2;
(c) a polynucleotide sequence which encodes a polypeptide that is 90% identical to the amino acid sequence of SEQ ID NO:2; and
(d) a fragment of a polynucleotide sequence of (a), (b), or (c); and
administering a treatment that reduces fat deposition to a subject from whom the sample originated where the presence of a polymorphic variation associated with fat reduction is detected in the PLA2G1B nucleotide sequence.

9. The method of claim 8, wherein the polymorphic variant is a guanine at position 7328, a thymine at position 9182, or a guanine at position 7328 and a thymine at position 9182.

10. The method of claim 8, wherein the treatment is one or more selected from the group consisting of an appetite suppressant, a lipase inhibitor, a phospholipase inhibitor, an exercise regimen, a dietary regimen, psychological consoling, psychotherapy, and a psychotherapeutic.

11. A method for reducing fat deposition in a subject, which comprises administering to a subject a molecule that inhibits a PLA2G1B polypeptide in the digestive tract of the subject, whereby inhibition of the PLA2G1B polypeptide in the digestive tract of the subject reduces fat deposition in the subject.

12. A method for reducing fat deposition in a subject, which comprises administering to a subject a molecule that inhibits a PLA2G1B polypeptide, wherein the subject does not experience significant steatorrhea after the molecule is administered, whereby inhibition of the PLA2G1B polypeptide reduces fat deposition in the subject.

13. A method for reducing fat deposition in a subject, which comprises administering to a subject a molecule that inhibits a PLA2G1B polypeptide, wherein the molecule induces less steatorrhea in subjects as compared to steatorrhea caused in subjects by a lipase inhibitor, whereby inhibition of the PLA2G1B polypeptide reduces fat deposition in the subject.

14. A method for identifying a candidate therapeutic for alleviating NIDDM, which comprises:
(a) introducing a test molecule to a system which comprises a nucleic acid comprising a PLA2G1B nucleotide sequence selected from the group consisting of:
(i) the nucleotide sequence of SEQ ID NO:1;
(ii) a nucleotide sequence which encodes a polypeptide consisting of the amino acid sequence of SEQ ID NO:2;
(iii) a nucleotide sequence which encodes a polypeptide that is 90% identical to the amino acid sequence of SEQ ID NO:2; and
(iv) a fragment of a nucleotide sequence of (i), (ii), or (iii); or
introducing a test molecule to a system which comprises a protein encoded by a nucleotide sequence of (i), (ii), (iii), or (iv); and
(b) determining the presence or absence of an interaction between the test molecule and the nucleic acid or protein,
whereby the presence of an interaction between the test molecule and the nucleic acid or protein identifies the test molecule as a candidate therapeutic for treating NIDDM.

15. The method of claim 14, wherein the system is an animal.

16. The method of claim 14, wherein the system is a cell.

17. The method of claim 14, wherein the PLA2G1B nucleotide sequence comprises a cytosine at position 7256 of SEQ ID NO:1.

18. A method for treating NIDDM in a subject, which comprises administering a candidate therapeutic of claim 14 to the subject in need thereof, whereby the candidate therapeutic treats NIDDM in the subject.

19. A method for alleviating NIDDM in a subject, which comprises contacting a PLA2G1B nucleic acid with one or more cells of a subject in need thereof, wherein the PLA2G1B nucleic acid comprises a nucleotide sequence selected from the group consisting of:
(a) the nucleotide sequence of SEQ ID NO:1;
(b) a nucleotide sequence which encodes a polypeptide consisting of the amino acid sequence of SEQ ID NO:2;
(c) a nucleotide sequence which encodes a polypeptide that is 90% identical to the amino acid sequence of SEQ ID NO:2; and
(d) a fragment of a nucleotide sequence of (a), (b), or (c);
whereby contacting the one or more cells of the subject with the PLA2G1B nucleic acid alleviates NIDDM.

20. A method for alleviating NIDDM in a subject, which comprises contacting a PLA2G1B protein with one or more cells of a subject in need thereof, wherein the PLA2G1B protein is encoded by a PLA2G1B nucleotide sequence which comprises a polynucleotide sequence selected from the group consisting of:
(a) the polynucleotide sequence of SEQ ID NO:1;
(b) a polynucleotide sequence which encodes a polypeptide consisting of the amino acid sequence of SEQ ID NO:2;
(c) a polynucleotide sequence which encodes a polypeptide that is 90% identical to the amino acid sequence of SEQ ID NO:2; and
(d) a fragment of a polynucleotide sequence of (a), (b), or (c);
whereby contacting the one or more cells of the subject with the PLA2G1B protein alleviates NIDDM.

21. A method for alleviating NIDDM in a subject, which comprises:
detecting the presence or absence of a polymorphic variant associated with NIDDM in a PLA2G1B nucleotide sequence in a nucleic acid sample from a subject, wherein the PLA2G1B nucleotide sequence comprises a polynucleotide sequence selected from the group consisting of:
(a) the polynucleotide sequence of SEQ ID NO:1;
(b) a polynucleotide sequence which encodes a polypeptide consisting of the amino acid sequence of SEQ ID NO:2;
(c) a polynucleotide sequence which encodes a polypeptide that is 90% identical to the amino acid sequence of SEQ ID NO:2; and
(d) a fragment of a polynucleotide sequence of (a), (b), or (c); and
administering a treatment that alleviates NIDDM to a subject from whom the sample originated where the presence of a polymorphic variation associated with NIDDM is detected in the PLA2G1B nucleotide sequence.

22. The method of claim 21, wherein the polymorphic variant is a cytosine at position 7256 of SEQ ID NO:1.

23. The method of claim 21, wherein the treatment is one or more selected from the group consisting of insulin, a hypoglycemic, a starch blocker, a liver glucose regulating agent, an insulin sensitizer, a glucose level monitoring regimen, dietary counseling, and a dietary regimen for managing blood glucose levels.
